(19) United States
(12) Patent Application Publication
(10) Pub. No.: US 2012/0183600 A1
(43) Pub. Date: Jul. 19, 2012

(54) NOVEL COMPOSITION FOR TREATING METABOLIC SYNDROME AND OTHER CONDITIONS

(76) Inventor: Chien-Hung Chen, New York, NY (US)

(21) Appl. No.: 13/343,634

(22) Filed: Jan. 4, 2012

Related U.S. Application Data
(63) Continuation-in-part of application No. 12/014,932, filed on Jan. 16, 2008.
(60) Provisional application No. 60/885,212, filed on Jan. 16, 2007.

(51) Int. Cl. A61K 31/616; A61K 9/127; A61P 35/00; A61P 3/04; A61P 9/12; A61P 3/08; A61K 31/415; A61P 3/00

(52) U.S. Cl. 424/450; 514/161; 514/406

(57) ABSTRACT

The invention relates to a composition that includes a first agent selected from the group consisting of an oxidative phosphorylation inhibitor, an ionophore, and an adenosine 5′-monophosphate-activated protein kinase (AMPK) activator; a second agent that possesses anti-inflammatory activity; and a third agent that possesses serotonin activity.

### CROSS-REFERENCES TO RELATED APPLICATION

This application is a continuation-in-part of U.S. patent application Ser. No. 12/014,932, by Chien-Hung Chen, entitled “Novel Composition for Treating Metabolic Syndrome,” filed on Jan. 16, 2008, which in turn claimed the benefit of U.S. Provisional Patent Application Ser. No. 60/885,212, by Chien-Hung Chen, entitled “Novel Composition for Treating Metabolic Syndrome,” filed on Jan. 16, 2007. The contents of these two prior applications are incorporated in their entirety by this reference. Additionally, this application is related to PCT Application Serial No. PCT/US2009/044362 by Chien-Hung Chen, entitled “Novel Compositions and Methods for Treating Hyperproliferative Diseases,” filed on May 18, 2009 and published as PCT Patent Application Publication No. WO 2009/140680 on Nov. 19, 2009, and to PCT Application Serial No. PCT/US2010/027330 by Chien-Hung Chen, entitled “Treating Alzheimer's Disease and Osteoporosis and Reducing Aging,” filed on Mar. 15, 2010 and published as PCT Patent Application Publication No. WO 2010/107702 on Sep. 23, 2010. The contents of these two prior PCT applications are incorporated in their entirety by this reference.

### BACKGROUND OF THE INVENTION

Metabolic syndrome is characterized by a group of metabolic risk factors, including abdominal obesity, atherogenic dyslipidemia (e.g., high triglyceride levels, low HDL cholesterol levels, and high cholesterol levels), hypertension, insulin resistance, prothrombotic state (e.g., high fibrinogen or plasminogen activator inhibitor-1 levels), and proinflammatory state (e.g., elevated C-reactive protein levels). Metabolic syndrome has become increasingly common in the United States. It is estimated that over 50 million Americans have this disorder. There is a need to develop novel drugs to effectively treat this disorder.

According to the World Health Organization, about five million people die from cancer every year. Drug treatment is one of the three major therapies for cancer. At present, drugs are used to treat cancers by the following mechanisms: interfering with or inhibiting cell division, regulating cell generation cycle, promoting tumor cell apoptosis, inhibiting angiogenesis, inhibiting oncogene activity, promoting tumor-suppressing gene activity, acting as tumor antigens, inhibiting telomerase activities, and interfering with information transfer of tumor cells.

In view of the high mortality rates associated with abnormal proliferative diseases including cancer, there exists a need for an effective treatment for these diseases.

Acquired immunodeficiency syndrome (AIDS), a consequence of infection with the HIV-1 retrovirus, affects over 30 million people worldwide. AIDS is characterized by a number of otherwise very rare opportunistic infections such as Kaposi's sarcoma, caused by the Kaposi's sarcoma-associated herpes virus, Pneumocystis jirovecii pneumonia, and other malignancies and infectious diseases. Patients with AIDS also suffer from severe weight loss, night sweats, swollen lymph nodes, and other consequences of a compromised immune system. In AIDS, CD4⁺ T cells are attacked by the virus and greatly reduced in number. Although treatments for AIDS do exist, including treatment with a “cocktail” of three drugs belonging to at least two classes of antiretroviral drugs, such as, for example, two nucleoside analogue reverse transcriptase inhibitors plus either a protease inhibitor or a non-nucleoside reverse transcriptase inhibitor. Although this approach has proved reasonably successful in inhibiting the growth of HIV-1 and preventing the occurrence of opportunistic infections and other symptoms of AIDS, it is not a cure and the effectiveness of drug therapy can be limited by drug resistance, drug toxicity, and possible patient non-compliance. Therefore, there is a need for an improved therapy for AIDS.

### SUMMARY OF THE INVENTION

This invention is based on the unexpected discovery that a combination of certain known drugs exhibits synergistic effects in treating metabolic syndrome and various other diseases. In addition to metabolic syndrome and diseases and conditions associated with metabolic syndrome, the combination of these known drugs can be used to treat hyperproliferative disease (including cancer), AIDS, Parkinson's disease, polycystic ovarian syndrome, Alzheimer's disease, osteoporosis, sleep apnea, erectile dysfunction, McArdle disease, and carbohydrate metabolism disorders. The combination of these known drugs can also be used to treat aging or fatigue. The combination of these known drugs can also be used to treat a disease or condition such as: (1) cardiac dysrhythmias; (2) endometriosis, uterine fibroid (uterine leiomyomata) menorrhagia, cervical erosion, cervical polyp, and related conditions; and (3) defects or disorders of intervertebral discs.

In one aspect, the invention features a composition that includes a first agent that can be an oxidative phosphorylation inhibitor, an ionophore, or an adenosine 5′-monophosphate-activated protein kinase (AMPK) activator, a second agent that possesses anti-inflammatory activity, and a third agent that possesses or maintains serotonin activity. The term “oxidative phosphorylation inhibitor” refers to any suitable agents that inhibit oxidative phosphorylation, such as oxidative phosphorylation uncouplers. An ionophore is a lipid-soluble molecule capable of transporting an ion across the lipid bilayer of cell membranes, and an AMPK activator is an agent that activates AMPK to phosphorylate its substrates, e.g., acetyl-CoA carboxylase and malonyl-CoA decarboxylase. Examples of the first agent include metformin (e.g., metformin chloride), phenformin, buformin, ephedrine, thyroxine, salicylanilide, and salicylic acid. The second agent can be any suitable anti-inflammatory compounds (e.g., non-steroidal anti-inflammatory compounds). Examples include aspirin (acetylsalicylic acid), diclofenac (e.g., diclofenac potassium or diclofenac sodium), ibuprofen (e.g., dexibuprofen or dexibuprofen lysine), indomethacin, acetaminophen, nimesulide, and a COX-2 inhibitor (e.g., a nitric oxide-based COX-2 inhibitor). The third agent can be a compound possessing or maintaining at least one of the serotonin's activities and, when used in combination with the first and second agents, effectively treats one or more of the target diseases of this invention. Examples includes serotonin (e.g., serotonin sulfate, a serotonin creatinine sulfate complex, or serotonin hydrochloride) and a serotonin re-uptake inhibitor. A preferred composition contains metformin hydrochloride, aspirin, and a serotonin creatinine sulfate complex. The three agents mentioned above can treat the target diseases via biological mechanisms other than those described therein. For example, metformin may treat a target disease (e.g., diabetes) via a mechanism other than inhibiting oxidative phosphorylation or activating AMPK.

In another aspect, the invention features a composition consisting essentially of a first agent that can be an oxidative phosphorylation inhibitor, an apoptogen, an ionophore, or an AMPK activator, a second agent that possesses anti-inflammatory activity, and a third agent that possesses serotonin activity. The term “consisting essentially of” used herein limits a composition to the three specified agents and those that do not materially affect its basic and novel characteristics, i.e., the efficacy in treating a target disease described herein. An example of such a composition contains the above-mentioned three agents and a pharmaceutically acceptable carrier.

The compositions described above can contain 5-5,000 mg (e.g., 5-3,000 mg, 5-1,500 mg or 5-1,000 mg) of the first agent, 1-5,000 mg (e.g., 1-3000 mg, 1-1,000 mg, 1-500 mg, or 1-100 mg) of the second agent, and 0.1-1,000 mg (e.g., 0.1-100 mg, 0.1-50 mg, or 0.1-30 mg) of the third agent, or in quantities of the same ratio as that calculated based on the above amounts.

In still another aspect, this invention features a method for treating metabolic syndrome, Parkinson's disease, or polycystic ovarian syndrome. The method includes administering to a subject in need thereof an effective amount of one or more of the compositions described above. The diseases mentioned above also include their associated disorders. For example, disorders associated with metabolic syndrome include atherosclerosis, coronary heart disease, stroke, obesity, diabetes, atherogenic dyslipidemia (e.g., high triglyceride levels, low HDL cholesterol levels, and high LDL cholesterol levels), hypertension, insulin resistance, prothrombotic state (e.g., high fibrinogen or plasminogen activator inhibitor-1 levels), and proinflammatory state (e.g., elevated C-reactive protein levels).

The term “treating” or “treatment” used herein refers to administering one or more above-described compositions to a subject, who has a disease described above, a symptom of such a disease, or a predisposition toward such a disease, with the purpose to confer a therapeutic effect, e.g., to cure, relieve, alter, affect, ameliorate, or prevent the disease, the symptom of it, or the predisposition toward it.

The composition described above can be in dry form (e.g., powder or tablet) or in aqueous form (e.g., beverage or syrup). It can be a dietary supplement or a pharmaceutical formulation (containing a pharmaceutically acceptable carrier). It can also be a drink or a food product. Examples include tea (e.g., a tea drink and the contents of a tea bag), soft drinks, juice (e.g., a fruit extract and a juice drink), milk, coffee, cookies, cereals, chocolates, and snack bars.

The first, second, and third agents described above include active compounds, as well as their salts, prodrugs, and solvates, if applicable. A salt, for example, can be formed between an anion and a positively charged group (e.g., amino) on an agent. Suitable anions include chloride, bromide, iodide, sulfate, nitrate, phosphate, citrate, methanesulfonate, trifluoroacetate, acetate, chlorophenyoxyacetate, malate, tosylate, tartrate, fumarate, glutamate, glucuronate, lactate, glutarate, benzoate, embonate, glycolate, pamoate, aspartate, parachlorophenoxyisobutyrate, formate, succinate, cyclohexanecarboxylate, hexanoate, octanoate, decanoate, hexadecanoate, octadecanoate, benzenesulphonate, trimethoxybenzoate, paratoluenesulphonate, adamantanecarboxylate, glycoxylate, pyrrolidonecarboxylate, naphthalenesulphonate, 1-glucosephosphate, sulfite, dithionate, and maleate. Likewise, a salt can also be formed between a cation and a negatively charged group (e.g., carboxylate) on an agent. Suitable cations include sodium ion, potassium ion, magnesium ion, calcium ion, and an ammonium cation such as tetramethylammonium ion. The agents also include salts containing quaternary nitrogen atoms. Examples of prodrugs include esters and other pharmaceutically acceptable derivatives, which, upon administration to a subject, are capable of providing active compounds. A solvate refers to a complex formed between an active compound and a pharmaceutically acceptable solvent. Examples of pharmaceutically acceptable solvents include water, ethanol, isopropanol, ethyl acetate, acetic acid, and ethanolamine.

Also within the scope of this invention is one or more compositions described above for use in treating an above-described disease, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

A composition of this invention can include three agents.

In general, the first agent is selected from the group consisting of an oxidative phosphorylation inhibitor, an apoptogen, an ionophore, and an adenosine 5′-monophosphate-activated protein kinase (AMPK) activator.

The first agent can include, in addition to those described above, 4,6-dinitro-o-cresol, uncoupling proteins (e.g., UCP1, UCP2, or UCP3), carbonyl cyanide p-(trifluoromethoxy)phenyl-hydrazone, carbonyl cyanide m-chlorophenyl-hydrazone, C5 gene products, dinitrophenol (e.g., 2,4-dinitrophenol), efrapeptin (A23871), guanethidine, chlorpromazine, amytal, secobarbital, rotenone, progesterone, antimycin A, naphthoquinone, 8-hydroxyquinoline, azides (e.g., NaN₃), dicoumarin, bilirubin, bile pigment, ephedrine, hydrogen sulfide, tetraiodothyronine, quercetin, 2,4-bis(p-chloroanilino)pyrimidine, glyceraldehyde-3-phosphate dehydrogenase, oligomycin, tributyltin chloride, aurovertin, rutamycin, venturicidin, dicyclohexylcarbodiimide, Dio-9, m-chlorophenyl-hydrazone mesoxalonitrile, ionomycin, calcium ionophores (e.g., A23187 (calcimycin), NMDA, CA 1001 ((−)-(R,R)—N,N′-bis[11-(ethoxycarbonyl)undecyl]-N,N′-4,5-tetramethyl-3,6-dioxaoctanediamide), or enniatin B), compounds that increase the Ca⁺² concentration in mitochondria (e.g., atractyloside, bongkrekic acid, thapsigargin, amino acid neurotransmitters, glutamate, N-methyl-D-aspartic acid, carbachol, ionophores, inducers of potassium depolarization), apoptogens (i.e., compounds that induce apoptosis, such as, but not limited to, 6-[3-adamantyl-4-hydroxyphenyl]-2-naphthalene carboxylic acid and fenretinide), valinomycin, gramicidin, nonactin, nigericin, lasalocid, and monensin. These compounds fall into the general categories of: (1) oxidative phosphorylation inhibitors; (2) apoptogens; or (3) ionophores.

In another alternative, the first agent can be an AMPK activator. AMPK activators include, but are not limited to: (1) metformin; (2) phenformin; (3) buformin; (4) AICAR; (5) thienopyridones; (6) resveratrol; (7) nootkatone; (8) thiazole; (9) adiponectin; (10) 2-deoxyglucose; (11) AAPDs (atypical antipsychotic drugs, including olanzapine, quetiapine, and risperidone); (12) adiponectin variant polypeptides such as AdipoR3v1 polypeptide, AdipoRe polypeptide, and AdipoR2vs polypeptide, disclosed in U.S. Pat. No. 7,435,808 to Wu et al., incorporated herein by this reference; (13) catechins, including catechin, gallocatechin, catechin gallate, gallocatechin gallate, epicatechin, epigallocatechin, epicatechin gallate and epigallocatechin gallate, disclosed in United States Patent Application Publication No. 2007/0004650 by Shimotoyodome et al., incorporated herein by this reference; (14) trans-10, cis-12 conjugated linoleic acid; (15) corydaline and related compounds, including corlumidin, (+)-corlumidin, corypalmine, 14R-(+)-corypalmine, tetrahydropalmatine, 14R-(+)-tetrahydropalmatine, 14R,13S-(+)-corydaline, bicuculline, d-(+)-bicuculline, egenine, and +-egenine, disclosed in United States Patent Application Publication No. 2009/0042810 by Chung and United States Patent Application Publication No. 2009/048246 by Lin et al., both of which are incorporated herein by this reference; (16) dithiolethiones, including oltipraz and 5-(4-methoxyphenyl)-3H-1,2-dithiole-3-thione; (17) inhibitors or antagonists of DNA-dependent protein kinase catalytic subunit (DNA-PKcs), disclosed in United States Patent Application Publication No. 2010/0130597 by Chung et al., incorporated herein by this reference; (18) small interfering RNAs (siRNAs) that can inhibit the expression and/or translation of DNA-PKcs, disclosed in United States Patent Application Publication No. 2010/0130597 by Chung et al., incorporated herein by this reference; (19) fibrates, including bezafibrate, ciprofibrate, fenofibrate, clofibrate, and gemfibrozil; (20) GW2974 (N-4-(1-benzyl-1H-indazol-5-yl)-N6,N6-dimethyl-pyrido-[3,4-d]-pyrimidine-4,6-diamine); (21) honokiol; (22) leptin; (23) LKB1 (serine/threonine kinase 11); (24) obovatol (4′,5-diallyl-2,3-dihydroxybiphenyl ether); (25) pioglitazone and related thiazolidinediones, including rosiglitazone and rosiglitazone maleate; (26) Y122S/1125E and additional muteins of adiponectins, disclosed in U.S. Pat. No. 7,678,886 to Zalevsky et al., incorporated herein by this reference, such as a variant adiponectin peptide with the formula: V(109)-V(110)-V(111)-F(112)-F(113-121)-V(122)-F(123)-V(124)-V(125)-F(126-127)-V(128)-F(129-134)-V(135)-F(136-151)-V(152)-F(153-163)-F-(164)-F(165-181)-V(182)-F(183)-V(184)-F(185-206)-V(207)-F(208-220)-F(221)-F(222-223)-V(224)-V(225)-F(226)-V(227)-F(228)-V(229), wherein V(109) is selected from the group consisting of: the wild-type amino acid V; any of variant amino acids D, E, H, K, N, Q, and R; and, a deletion of V109; V(110) is selected from the group consisting of: the wild-type amino acid V; any of variant amino acids D, E, H, K, N, Q, R, and S; and, a deletion of V110; V(111) is selected from the group consisting of: the wild-type amino acids Y and H; any of variant amino acids D, E, N, R, and S; and, a deletion of 111; F(112) is selected from the group consisting of the wild-type amino acids R and C, and, a deletion of 112; F(113-121) is selected from the group consisting of: the wild-type amino acid sequence SAFSVGLET (SEQ ID NO: 1); and, a deletion of any of S113, A114, F115, S116, V117, G118, L119, E120, and T121; V(122) is selected from the group consisting of: the wild-type amino acid Y; any of variant amino acids D, E, H, N, R, and S; and, a deletion of Y122; F(123) is selected from the group consisting of: the wild-type amino acid sequence V and a deletion of V123; V(124) is selected from the group consisting of: the wild-type amino acid T; any of variant amino acids D, E, K, N, and R; and, a deletion of 1124; V(125) is selected from the group consisting of: the wild-type amino acid I; any of variant amino acids D, E, H, K N, Q, R, S, and T; and, a deletion of I125; F(126-127) comprises the wild-type amino acid sequence PN; V(128) is selected from the group consisting of: the wild-type amino acid M; and any of variant amino acids A, D, E, H, K, N, Q, R, S, and T; F(129-134) comprises the wild-type amino acid sequence PIRFTK (SEQ ID NO: 2); V(135) is selected from the group consisting of: the wild-type amino acid I; and, any of variant amino acids D, E, H, K, N, Q and R; F(136-151) comprises the wild-type amino acid sequence FYNQQNHYDGSTGKFH (SEQ ID NO: 3); V(152) is selected from the group consisting of: the wild-type amino acid C; and, any of variant amino acids A, F, L, N, S, T and V; F(153-163) comprises the wild-type amino acid sequence NIPGLYYFAYH (SEQ ID NO: 4); F(164) is selected from the group consisting of the wild-type amino acid I and T; F(165-181) comprises the wild-type amino acid sequence TVYMKDVKVSLFKKDKA (SEQ ID NO: 5); V(182) is selected from the group consisting of: the wild-type amino acid M; and, any of variant amino acids A, D, E, K, N, Q, R, S, and T; F(183) comprises the wild-type amino acid L; V(184) is selected from the group consisting of: the wild-type amino acid F; and, any of variant amino acids D, H, K, N and R; F(185-206) comprises the wild-type amino acid sequence TYDQYQENNVDQASGSVLLHLE (SEQ ID NO: 6); V(207) is selected from the group consisting of: the wild-type amino acid V; and, any of variant amino acids D, E, H, K, N, Q, R, and S; F(208-220) comprises the wild-type amino acid sequence GDQVWLQVYGEGE (SEQ ID NO: 7); F(221) is selected from the group consisting of the wild-type amino acids R and S; F(222-223) comprises the wild-type amino acid sequence NG; V(224) is selected from the group consisting of: the wild-type amino acid L; and, any of variant amino acids D, E, H, K, N, Q, R and S; V(225) is selected from the group consisting of: the wild-type amino acid Y; and, any of variant amino acids D, E, H, K, N, Q, R and S; F(226) comprises the wild-type amino acid A; V(227) is selected from the group consisting of: the wild-type amino acid D; and, any of variant amino acids H, K and R; F(228) comprises the wild-type amino acid N; or V(229) is selected from the group consisting of: the wild-type amino acid D; and, any of variant amino acids H, K and R, the variant adiponectin having at least threefold increased solubility when compared to wild-type adiponectin; (27) butyrate and butyrate analogs as disclosed in United States Patent Application Publication No. 2011/0077300 by Ye et al., incorporated herein by this reference, including, but not limited, to butyrate salts, including sodium butyrate, butyl butyrate, n-pentyl butyrate, isobutyl butyrate, α-methylbenzyl butyrate, hexyl butyrate, phenethyl butyrate, methyl butyrate, ethyl butyrate, 2-hydroxy-3-methylbutanoic acid, trimethylbutyrin, a triglyceride with at least one butyrate moiety attached to the glycerol backbone of the triglyceride, preferably two butyrate moieties attached to the glycerol backbone of the triglyceride, wherein the triglyceride also comprises at least one long-chain fatty acid attached to the glycerol backbone of the triglyceride, wherein the long-chain fatty acid is a saturated fatty acid or an unsaturated fatty acid, and in which a preferred long-chain fatty acid is oleate; (28) quinoxalinedione derivatives as described in United States Patent Application Publication No. 2011/0130404 by Cravo et al., incorporated herein by this reference; (29) thienopyridone derivatives as described in United States Patent Application Publication No. 2011/0034505 by Cravo et al., incorporated herein by this reference; and (30) thienopyridone derivatives as described in United States Patent Application Publication No. 2011/0006001 by Cravo et al., incorporated herein by this reference; and the salts, solvates, analogues, congeners, bioisosteres, hydrolysis products, metabolites, precursors, and prodrugs thereof. It is generally preferred that the first agent is an AMPK activator.

In another alternative, the first agent can be selected from the group consisting of ephedrine, thyroxine, salicylanilide, or salicylic acid; and the salts, solvates, analogues, congeners, bioisosteres, hydrolysis products, metabolites, precursors, and prodrugs thereof.

The second agent can include steroidal anti-inflammatory drugs or non-steroidal anti-inflammatory drugs.

Typically, steroidal anti-inflammatory drugs suitable for use in compositions and methods of the present invention are glucocorticoids, or steroids that have glucocorticoid activity. Such steroids may also have a certain degree of mineralocorticoid activity, but anti-inflammatory activity of steroidal anti-inflammatory drugs is closely associated with their glucocorticoid activity.

Ether derivatives of the steroid dexamethasone are disclosed in U.S. Pat. No. 5,223,493. These derivatives include, but are not limited to, 9α-fluoro-11β,17α-dihydroxy-21-methoxy-16α-methylpregna-1,4-diene-3,20-dione, 9α-fluoro-11β,17α-dihydroxy-21-benzyloxy-16α-methylpregna-1,4-diene-3,20-d lone, 9α-fluoro-11β,17α-dihydroxy-21-(2-methoxyethoxy)methoxy-16α-methylpregna-1,4-diene-3,20-dione, 9α-fluoro-11β,17α-dihydroxy-21-(2-hydroxylethoxy)-16α-methylpregna-1,4-diene-3,20-dione, 9α-fluoro-11β,17α-dihydroxy-21-(methylthiomethoxy)-16α-methylpregna-1,4-diene-3,20-dione, 9α-fluoro-11β,17α-dihydroxy-21-(methoxy)methoxy-16α-methylpregna-1,4-diene-3,20-dione, 9α-fluoro-11β,17α-dihydroxy-Δ₂₀-ethoxy-21-ethoxy-16α-methylpregna-1,4-diene-3,20-dione, 9α-fluoro-11β,17α-dihydroxy-21-ethoxy-16α-methylpregna-1,4-diene-3,20-dione, 9α-fluoro-11β,17α-dihydroxy-21-allyloxy-16α-methylpregna-1,4-diene-3,20-dione, 9α-fluoro-11β,17α-dihydroxy-21-cyclopropylmethoxy-16α-methylpregna-1,4-diene-3,20-dione, 9α-fluoro-11β,17α-dihydroxy-21-allyl-21-allyloxy-16α-methyl-1,4-diene-3,20-dione, 9α-fluoro-11β,17α-hydroxy-21-isopropyloxy-16α-methylpregna-1,4-diene-3,20-dione, 9α-fluoro-11β-propionoxy-17α-hydroxy-21-methoxy-16α-methylpregna-3,20-dione, and 9α-fluoro-11β-17α-diacetoxy-21-methoxy-16α-methylpregna-1,4-diene-3,20-dione.

Reactions of steroids are well known in the art, and need not be described further here. Many steroids undergo esterification at one or more hydroxyl residues with an acyl radical to form ester derivatives. Ether derivatives of steroids can be formed by the Williamson ether synthesis or other ether-forming reactions known in the art. Steroids are also subject to halogenation and other modification reactions.

Examples of steroidal anti-inflammatory drugs include: (1) hydrocortisone (including esters such as hydrocortisone acetate, hydrocortisone butyrate, hydrocortisone cypionate, hydrocortisone sodium phosphate, hydrocortisone sodium succinate, and hydrocortisone valerate); (2) cortisone; (3) beclomethasone (including esters such as beclomethasone propionate, beclomethasone dipropionate; (4) betamethasone (including esters such as betamethasone dipropionate, betamethasone sodium phosphate, and betamethasone valerate); (5) dexamethasone (including esters such as dexamethasone acetate and dexamethasone sodium phosphate); (6) prednisone; (7) methylprednisolone (including esters such as methylprednisolone acetate and methylprednisolone sodium succinate); (8) triamcinolone (including acetonide derivatives such as triamcinolone acetonide and triamcinolone hexacetonide and other derivatives such as triamcinolone benetonide as well as esters such as triamcinolone diacetate); (9) fluocinolone (including acetonide derivatives such as fluocinolone acetonide); (10) fludrocortisone (including esters such as fludrocortisone acetate); (11) hyaluronic acid 6-methylprednisolone ester; (12) rimexolone; (13) deflazacort, (14) prednisolone (including esters such as prednisolone farnesylate, prednisolone acetate, prednisolone sodium phosphate, prednisolone 25-diamino-acetate, and prednisolone tebutate); (15) ORG6632 (21-chloro-9α-11β-hydroxy-16α,17α-dimethylpregna-1,4-diene-3,20-dione); (16) 21-acetoxypregnenolone, (17) alclometasone; (18) algestone; (19) amcinonide; (20) azulfidine; (21) budesonide; (22) chloroprednisone; (23) clobetasol (including esters such as clobetasol propionate); (24) clocortolone (including esters such as clocortolone pivalate); (25) cioprednol; (26) corticosterone; (27) desonide; (28) desoximetasone; (29) desoxycorticosterone (including esters such as desoxycorticosterone acetate); (30) diflorasone; (31) difluprednate; (32) enoxolone; (33) fluazacort; (34) flucloronide; (35) flumethasone; (36) flunisolide; (37) fluocortolone; (38) fluorometholone; (39) fluprednidene (including esters such as fluprednidene acetate); (40) fluprednisolone; (41) fluticasone (including esters such as fluticasone propionate); (42) halcinonide; (43) halobetasol (including esters such as halobetasol propionate); (44) halometasone; (45) hydrocortamate; (46) medrysone; (47) meprednisone; (48) mometasone (including esters such as mometasone furoate); (49) paramethasone; (50) prednicarbate; (51) prednival; (52) prednylidene; (53) tixocortol; (54) clobetasone; (55) cortivazol; (56) diflucortolone; (57) fluocinolone (including acetonide derivatives such as fluocinolone acetonide); (58) fluocortin (including esters such as fluocortin butyl); (59) fluperolone (including esters such as fluperolone acetate); (60) formocortal; (61) halopredone (including esters such as halopredone acetate); (62) mazipredone; (63) 6α,9α-difluoro-17α-[(2-furanylcarbonyl)oxy]-11β-hydroxy-16α-methyl-3-oxoandrosta-1,4-diene-17β-carbothioic acid S-fluoromethyl ester; (64) 6α,9α-difluoro-11β-hydroxy-16α-methyl-3-oxo-17α-propionyloxy-androsta-1,4-diene-17β-carbothioic acid S-(2-oxo-tetrahydrofuran-3S-yl) ester; (65) rofleponide; (66) ciclesonide; (67) butixocort (including esters such as butixocort propionate); (68) RPR-106541 (20R-16α,17α-[butylidenebis(oxy)]-6α,9α-difluoro-11β-hydroxy-17β-(methylthio)androsta-4-en-3-one); (69) ST-126 (9-Fluoro-11β,17α-trihydroxy-16α-methyl-1,4-pregnadiene-3,20-dione 21-cyclohexanecarboxylate cyclopropanecarboxylate); (70) flurandrenolide; (71) 9α-fluoro-11β,17α-dihydroxy-21-methoxy-16α-methylpregna-1,4-diene-3,20-dione; (72) 9α-fluoro-11β,17α-dihydroxy-21-benzyloxy-16α-methylpregna-1,4-diene-3,20-dione; (73) 9α-fluoro-11β, 17α-dihydroxy-21-(2-methoxyethoxy)methoxy-16α-methylpregna-1,4-diene-3,20-dione; (74) 9α-fluoro-11β,17α-dihydroxy-21-(2-hydroxylethoxy)-16α-methylpregna-1,4-diene-3,20-dione; (75) 9α-fluoro-11β,17α-dihydroxy-21-(methylthiomethoxy)-16α-methylpregna-1,4-diene-3,20-dione (76) 9α-fluoro-11β,17α-dihydroxy-21-(methoxy)methoxy-16α-methylpregna-1,4-diene-3,20-dione; (77) 9α-fluoro-11β,17α-dihydroxy-Δ₂₀-ethoxy-21-ethoxy-16α-methylpregna-1,4-diene-3,20-dione; (78) 9α-fluoro-11β,17α-dihydroxy-21-ethoxy-16α-methylpregna-1,4-diene-3,20-dione; (79) 9α-fluoro-11β,17α-dihydroxy-21-allyloxy-16α-methylpregna-1,4-diene-3,20-dione; (80) 9α-fluoro-11β,17α-dihydroxy-21-cyclopropylmethoxy-16α-methylpregna-1,4-diene-3,20-dione; (81) 9α-fluoro-11β,17α-dihydroxy-21-allyl-21-allyloxy-16α-methyl-1,4-diene-3,20-dione; (82) 9α-fluoro-11β,17α-hydroxy-21-isopropyloxy-16α-methylpregna-1,4-diene-3,20-dione; (83) 9α-fluoro-11β-propionoxy-17α-hydroxy-21-methoxy-16α-methylpregna-3,20-dione; and (84) 9α-fluoro-11β-17α-diacetoxy-21-methoxy-16α-methylpregna-1,4-diene-3,20-dione; and the esters, acetonides, benetonides, furetonides, salts, solvates, analogues, congeners, bioisosteres, hydrolysis products, metabolites, precursors, and prodrugs thereof.

Examples of non-steroidal anti-inflammatory drugs (NSAIDs) include: (1) A183827; (2) ABT963 ((2-(3,4-difluoro-phenyl)-4-(3-hydroxy-3-methyl-butoxy)-5-(4-methanesulfonyl-phenyl)-2H-pyridazin-3-one); (3) aceclofenac; (4) acemetacin; (5) acetaminophen; (6) acetylsalicylic acid; (7) ACP (4-[bis(acetyloxy)methyl]-1,2-benzenediol diacetate); (8) actarit (4-(acetylamino)phenylacetic acid); (9) AHR10037 (2-amino-3-(4-chlorobenzoyl)benzeneacetamide); (10) AH R15010 (1-[(2-methoxyphenoxy)methyl-1,2-ethanediyl ester of sulfamic acid) (11) alclofenac; (12) alminoprofen; (13) amfenac; (14) ampiroxicam (15) amtolmetin guacil; (16) apazone; (17) araprofen; (18) atliprofen methyl ester; (19) AU8001 (4′-acetamidophenyl-2-(5′-4-tolyl-1-methylpyrrole)acetate); (20) azapropazone; (21) bendazac; (22) benoxaprofen; (23) benzydamine; (24) benzydamine flufenamate; (25) bermoprofen; (26) benzpiperylon; (27) BF388 (1-(3,5-di-cert-butyl-4-hydroxyphenyl)pyrrolidin-2-one); (28) BF389 (dihydro-4-[[3,5-bis(1,1-dimethyl)-4-hydroxyphenyl]methylene]-2-methyl-2H-1,2-oxazin-3(4H)-one); (29) BIRL790 (6-chloro-4-[(1-methylethyl)sulfonyl]-2-(phenylmethyl)-1,3(2H,4H)-isoquinolinedione); (30) BMS347070 ((Z)-3-(1-(4-bromophenyl)-1-(4-methylsulfonylphenyl)methylidine)-dihydrofuran-2-one, a COX-2 inhibitor); (31) bromfenac; (32) bucloxic acid; (33) bumadizone; (34) butibufen; (35) BW4C((N-(3-phenoxy-phenyl-2-propenyl)acetohydroxamic acid); (36) BW755C ((3-amino-1-[m-(trifluoromethyl)phenyl]-2-pyrazoline); (37) C53; (38) C73; (39) C85; (40) carprofen; (41) CBS1108 (2-acetylthiophene-2-thiazolylhydrazone); (42) celecoxib; (43) CGS25997 ((2S)-(−)-2-[[N-(aminocarbonyl)-N-hydroxyamino]methyl-7-fluoroxyphenyl-1,4-benzodioxan); (44) CHF2003; (45) chlorobiphenyl; (46) choline magnesium trisalicylate; (47) CHX108 (a lipoxygenase/cyclooxygenase inhibitor); (48) C1959 (5-methoxy-3-(1-methylethoxy)-N-1H-tetrazol-5-yl-benzo9b]thiophene-2-carboxamide sodium salt); (49) cimicoxib; (50) cinmetacin; (51) cinnoxicam; (52) clidanac; (53) clofezone; (54) clonixin; (55) clopirac; (56) CLX1205; (57) COX-2 inhibitors; (58) CP331 (N-(3-[3-(piperidinyl-methyl)phenoxy]propyl)-carbamoyl-methylthio]ethyl 1-(p-chlorobenzoyl) 5-methoxy-2-methyl-3-indolyl-acetate); (59) CS502 (a COX-2 inhibitor); (60) CS706 (2-(4-ethoxyphenyl)-4-methyl-1-(4-sulfamoylphenyl)-1H-pyrrole); (61) D1367 (a COX-2 inhibitor); (62) darbufelone; (63) deracoxib; (64) dexibuprofen; (65) dexibuprofen lysine; (66) dexketoprofen; (67) DFP; (68) DFU ((5,5-dimethyl-3-(3-fluorophenyl)-4-(4-methylsulphonyl)phenyl-2(5H)-furanone); (69) diclofenac sodium; (70) diclofenac potassium; (71) diflunisal; (72) DP155 (mixture of 1-steroyl and 1-palmitoyl-2-{4-[1-(p-chlorobenzoyl)-5-methoxy-2-methyl-3-indolylacetamido]hexanoyl}-sn-glycero-3-phosphatidyl choline); (73) DRF4367 (2-hydroxymethyl-4-(5-(4-methoxyphenyl)-3-trifluoromethyl-1H-1-pyrazolyl)-1-benzenesulfonamide); (74) droxicam; (75) E5110 (N-methoxy-3-(3,5-di-tert-butyl-4-hydroxybenzylidene pyrrolidin-2-one); (76) E6080 (4-[[(6-hydroxy-4,4-7-trimethyl-2-benzothiazolyl)amino]methyl]benzenesulfonamide monohydrochloride); (77) E6087 (4-(5-(2,4-difluorophenyl)-4,5-dihydro-3-trifluoromethyl-1H-pyrazol-1-yl)benzenesulfonamide); (78) eltenac; (79) enfenamic acid; (80) epirizole; (81) ER34122 (5-[1-[1,5-bis(4-methoxyphenyl)pyrazol-3-yl]-1,1-dimethoxymethyl]-2-chlorobenzamide); (82) esflurbiprofen; (83) ethenzamide; (84) etodolac; (85) etofenamate; (86) etoricoxib; (87) F025; (88) FCE20696 ((6H-dibenzo[b,d]pyran-6-carboxylic acid 2-(dimethylamino)ethyl ester hydrochloride); (89) felbinac; (90) felbinac ethyl; (91) fenbufen; (92) fenclofenac; (93) fenclozic acid; (94) fenclozine; (95) fendosal; (96) fenoprofen; (97) fentiazac; (98) fepradinol (α-[[(2-hydroxy-1,1-dimethylethyl)amino]methyl]benzenemethanol); (99) feprazone; (100) filenadol; (101) flobufen; (102) florifenine; (103) flosulide; (104) flubichin methanesulfonate; (105) flufenamic acid; (106) flufenisal; (107) flunixin; (108) flunoxaprofen; (109) fluprofen; (110) fluproquazone; (111) flurbiprofen; (112) FPL62064 (N-(4-methoxyphenyl)-1-phenyl-1H-pyrazole-3-amine); (113) FR111142 (4,5-dihydroxy-2-hexenoic acid 5-methoxy-4-[2-methyl-3-(3-methyl-2-butenyl)oxiranyl]-1-oxaspiro[2.5]oct-6-yl ester); (114) FR122047 (1-[[4,5-bis(4-methoxyphenyl)-2-thiazolyl]carbonyl]-4-methylpiperazine hydrochloride; a COX-1 inhibitor); (115) FR123826 (a COX-2 inhibitor); (116) FR140423 (3-(difluoromethyl)-1-(4-methoxyphenyl)-5-[4-(methylsulfinyl)phenyl]pyrazole; a COX-2 inhibitor); (117) FR188582 (3-chloro-5-[4-(methylsulfonyl)phenyl]-1-phenyl-1H-pyrazole; a COX-2 inhibitor); (118) FS205397 (an analgesic); (119) furofenac; (120) GR80907; (121) GR129574A ((R)—N[1-carboxy-3-(1,3-dihydro-1,3-dioxo-2Hbenz[f]isoindol-2-yl)propyl]-L-leucyl-N-methyl-L-phenylalanamide); (122) GR253035 (a COX-2 inhibitor); (123) GW406381 (a COX-2 inhibitor); (124) HA1105; (125) HA1106; (126) HCT2035 (NO-ketoprofen or toprofen); (127) HGP12; (128) HN3392; (129) HP977 (3-(6,11-dihydro-11-oxodibenz(b,e)oxepin-2-yl)-N-hydroxy-N-methylpropanamide); (130) HX0835; (131) HYAL AT2101 (a topical gel of hyaluranon and 3% diclofenac); (132) ibufenac; (133) ibuprofen; (134) ibuproxam-beta-cyclodextrin; (135) icodulinum; (136) IDEA070 (a COX-1, COX-2, and lipoxygenase inhibitor); (137) iguratimod; (138) imrecoxib; (139) indomethacin; (140) indoprofen; (141) IP751 (ajulemic acid); (142) IRA378 ((S)-8-chloro-1,2,3,4-tetrahydro-2-(trifluoromethyl)-6-quinolineacetic acid); (143) isofezolac; (144) isoxepac; (145) isoxicam; (146) 1×207887 (10-methoxy-4H-benzo[4,5]cyclohepta(1,2-b)thiophene-4-yliden)acetic acid); (147) KC764 (2-methyl-3-(1,4,5,6-tetrahydronicotinoyl)pyrazolo[1,5-a]pyridine); (148) ketoprofen; (149) ketorolac; (150) L652343 (3-hydroxy-5-trifluoromethyl-N-[2-(2-thienyl)-2-phenyl-ethenyl]-benzo(B) thiophene-2-carboxamide); (151) L745337 (5-methanesulfonamido-6-(2,4-difluorothiophenyl)-1-indanone); (152) L748731 (a COX-2 inhibitor); (153) L752860 (5,5-dimethyl-4-(4-(methylsulfonyl)phenyl)-3-(3-fluorophenyl)-5H-furan-2-one); (154) L651392 (4-bromo-2,7-dimethoxy-3H-phenothiazin-3-one); (155) L663536 (3-[3-butylsulfanyl-1-[(4-chlorophenyl)methyl]-5-propan-2-yl-indol-2-yl]-2,2-dimethyl-propanoic acid); (156) L761066 (a COX-2 inhibitor); (157) L768277 (a substituted 5,6-diarylthiazolo[3,2-b][1,2,4]triazole; a COX-2 inhibitor); (158) L776967; (159) L783003; (160) L784520; (161) L791456 (5-chloro-2-methylpyridin-3-yl)-3-(4-methylsulfonylphenyl)pyridine, a COX-2 inhibitor); (162) L804600 (2-benzyl-4-isopropoxy-5-[4-(methylsulfonyl)phenyl]pyridazin-3(2H)-one); (163) L818571 (2-(cyclopropylmethyl)-4-(4-fluorophenyl)-5-[4-(methylsulfonyl)phenyl]pyridazin-3(2H)-one); (164) LAS33815 (4-(2,3-dihydro-2-oxo-3-phenyl-4-oxazolyl)-benzenesulfonamide); (165) LAS34475 (a COX-2 inhibitor); (166) licofelone; (167) LM4108 (([1-(4-chlorobenzoyl)-5-methoxy-2-methyl-1H-indol-3-yl]-N-phenethyl-acetamide or indomethacin phenethylamide); (168) lobuprofen; (169) lornoxicam; (170) lonazolac; (171) loxaprofen; (172) lumaricoxib; (173) LY221608 (5-[[3,5-bis(1,1-dimethylethyl)-4-hydroxyphenyl]methylene]-3-(dimethylamino)-4-thiazolidinone); (174) LY269415 (5-[[3,5-bis(1,1-dimethylethyl)-4-hydroxyphenyl]methylene]-3-(methylamino)-4-thiazolidinone); (175) mabuprofen; (176) meclofenamic acid; (177) meclofenamate sodium; (178) mefenamic acid; (179) meloxicam; (180) mercaptoethylguanidine; (181) mesaclazone; (182) mesoporphyrin; (183) metoxibutropate; (184) miroprofen; (185) mofebutazone; (186) mofezolac; (187) morazone; (188) MX1094 (a prodrug of naproxen); (189) nabumetone; (190) naproxen sodium; (191) naproxen sodium/metoclopramide; (192) NCX1101 (nitric oxide donor grafted to a conventional drug); (193) NCX284 (NO-diclofenac); (194) NCX285 (NO-diclofenac); (195) NCX4016; (196) NCX4215; (197) NCX530 (a nitric-oxide-releasing derivative of indomethacin, 1-(4-chlorobenzoyl)-5-methoxy-2-1H-indole-3-acetic acid 3-(nitrooxymethyl)phenyl ester)); (198) nepafanac; (199) niflumic acid; (200) nimesulide; (201) nitric oxide-based NSAIDs (NitroMed, Lexington, Mass.); (202) nitrofenac; (203) nitroflurbiprofen; (204) nitronaproxen; (205) NS398 (N-[2-cyclohexyloxy-4-nitrophenyl]methanesulfonamide); (206) ocimum sanctum oil; (207) olsalazine; (208) ONO3144 (2-amino-4-t-butyl-6-propionylphenol); (209) orpanoxin; (210) oxaceprol; (211) oxaprozin; (212) oxindanac; (213) oxpinac; (214) oxycodone/ibuprofen; (215) oxyphenbutazone; (216) P10294 (3-(6,11-dihydrodibenz[b,e]oxepin-2-yl)-N-hydroxy-N-methylpropanamide); (217) P54 (a phytochemical-based selective COX-2 inhibitor); (218) P8892 (a cyclooxygenase/lipoxygenase inhibitor); (219) pamicogrel; (220) parcetasal; (221) parecoxib; (222) parsalmide; (223) PD138387 ((Z)-5-(3,5-di-tert-butyl-4-hydroxybenzylidene)-2-(methoxyamino)thiazol-4(5H)-one, a COX-2 inhibitor); (224) PD145246; (225) PD164387 (2,6-di-tert-butyl-4-[5-(ethylsulfanyl)-1,3,4-thiadiazol-2-yl]phenol); (226) pelubiprofen; (227) pemedolac; (228) phenylbutazone; (229) pirazolac; (230) piroxicam; (231) piroxicam beta-cyclodextrin; (232) piroxicam pivalate; (233) pirprofen; (234) pranoprofen; (235) prinomide (α-cyano-1-methyl-b-oxopyrrole-2-propionanilide with 2-amino-2-(hydroxymethyl)-1,3-propanediol); (236) proglumetacin; (237) resveratrol; (238) R-ketoprofen; (239) R-ketorolac; (240) Ro323555 (β-(cyclopentylmethyl)-N-hydroxy-γ-oxo-α[(3,4,4-trimethyl-2,5-dioxo-1-imidazolidinyl)methyl]-1-piperidinebutanamide); (241) rofecoxib; (242) RP54745 (4-chloro-5-(3,4-dihydro-1-methyl-2(1H)-isoquinolinyl)-3H-1,2-dithiol-3-one); (243) RP66364 (2,4,5-(3-phenylpropyl)-2-thienylbutyoxyacetic acid; a LTB₄ antagonist); (244) RU43526 (a 4-hydroxy-3-quinolinecarboxamide); (245) RU46057 (2-[1-bis(1-oxopropoxyethyl]-4-hydroxy-N-2-thiazolyl-8-(trifluoromethyl)-3-quinoline carboxamide); (246) RU54808; (247) RWJ63556 (N-[5-(4-fluorophenoxy)thien-2-yl]methane sulfonamide; a dual COX-2 selective/5-lipoxygenase inhibitor); (248) S19812 (N-hydroxy-N-methyl-4-(2,3-bis-(4-methoxyphenyl)-thiophen-5-yl) butanamide, a dual inhibitor of cyclooxygenase and lipoxygenase); (249) S33516; (250) salicin; (251) salicylamide; (252) salicylsalicylic acid; (253) satigrel; (254) SC236 ME)-(5)-(3,5-di-tert-butyl-4-hydroxybenzylidene)-2-ethyl-1,2-isothiazolidine-1,1-dioxide, also known as S2474); (255) SC57666 (a selective COX-2 inhibitor); (256) SC58125 (5-(4-fluorophenyl)-1-[4-(methylsulfonyl)phenyl]-3-(trifluoromethyl)-1H-pyrazole, a selective COX-2 inhibitor); (257) SC58451 (a selective COX-2 inhibitor); (258) SD8381 (COX-2 inhibitor); (259) seprilose (3-O-heptyl-1,2-O-(1-methylethylidene)-α-D-glucofuranose); (260) SFPP; (261) SKF105809 (((2-4-methylsulfonylphenyl)-3-(4-pyridyl)-6,7-dihydro-[5H]-pyrrolo[1,2-a]imidazole); (262) SKF86002 (6-(4-fluorophenyl)-2,3-dihydro-5-(4-pyridinyl)imidazo[2,1-b]thiazole dihydrochloride, an inhibitor of p38 MAP kinase); (263) sodium salicylate; (264) sudoxicam; (265) sulfasalazine; (266) sulindac; (267) suprofen; (268) SVT2016 (5(R)-thiosulfonamide-3-(2H)-benzofuranone); (269) T3788 (1-(4-aminophenyl)-1-ethanone); (270) TA60 (2-[4-(3-methyl-2-butenyl)phenyl]propionic acid); (271) talmetacin; (272) talniflumate; (273) tazofelone; (274) tebufelone; (275) tenidap; (276) tenoxicam; (277) tepoxalin; (278) tiaprofenic acid; (279) tiaramide; (280) tilmacoxib; (281) tilnoprofen arbamel; (282) tinoridine; (283) tiopinac; (284) tioxaprofen; (285) tolfenamic acid; (286) tolmetin; (287) triflusal; (288) tropesin; (289) TY10222 (3-(((2-chloro(1,1-biphenyl)-4-yl)methoxy)methyl)-pyridine ethanedioate); (290) TY10246; (291) TY10474; (292) UR8962 (4-[4-(methylsulfonyl)phenyl]-3-[6-(1-pyrrolidinyl)pyridin-3-yl]furan-2(5H)-one); (293) U91502 ([3-(1,6-dihydro-1-methyl-6-oxo-4-phenyl-2-pyrimidinyl)propylidene]bisphosphonic acid tetraethyl ester); (294) ursolic acid; (295) valdecoxib; (296) WAY120739 (1,8-diethyl-1,3,4,9-tetrahydro-6-(2-quinolinylmethoxy)pyrano[3,4-b]indole-1-acetic acid; a dual inhibitor of 5-lipoxygenase and cyclooxygenase); (297) WY28342; (298) WY41770 ((5H-dibenzo[a,d]cyclohepten-5-ylidene)acetic acid); (299) WY46135 (N-[[(5-chloro-2-benzothiazolypthio]phenylacetyl]-L-cysteine); (300) ximoprofen; (301) YS134; (302) zaltoprofen; (303) ZD2138 (6-[[3-fluoro-5-(tetrahydro-4-methoxy-2H-pyran-4-yl)phenoxy]methyl]-1-methyl-2(1H)quinolinone); (304) zidometacin; (305) zomepirac; (306) AA961; (307) acetaminosalol; (308) AD1590 (2-(8-methyl-10,11-dihydro-11-oxodibenz[b,f]oxepin-2-yl) propionic acid); (309) AFP802; (310) aloxiprin; (311) amfenac sodium; (312) aminopropylon; (313) aminopyrine; (314) amoxiprin; (315) anirolac; (316) anitrazafen; (317) antrafenine; (318) 2-arylpropionic acids; (319) azulene sodium sulfonate; (320) baicalein; (321) bendazac lysinate; (322) benorylate; (323) biphenyl aspirin (2′-acetoxy-biphenyl-2-carboxylic acid); (324) BPPC; (325) bromfenac sodium; (326) broperamole; (327) bufexamac; (328) bufezolac; (329) BW540C; (330) caffeic acid; (331) calcium acetylsalicylate; (332) Chinoin 127; (333) choline salicylate; (334) cicloprofen; (335) cinchophen; (336) cintazone; (337) cipamfylline; (338) clobuzarit; (339) clometacin; (340) clonixeril (2,3-dihydroxypropyl 2-(3-chloro-o-toluidino)nicotinate); (341) cloximate; (342) CN 100 (2-(10,11-dihydro-10-oxo-dibenzo[b,f]thiepin-2-yl)propionic acid); (343) 4-(4-cyclohexyl-2-methyloxazol-5-yl)-2-fluorobenzenesulfonamide; (344) cyclooxygenase-1 inhibitors; (345) delmetacin (UR2310 or 1-benzoyl-2-methylindole-3-acetic acid); (346) dexindoprofen; (347) diaryl-5-oxygenated-2-(5H)-furanone; (348) 2,4-dichlorobenoxaprofen; (349) difenpiramide; (350) diflumidone sodium; (351) 2-(3,5-difluorophenyl)-3-[4-(methylsulfonyl)phenyl]-2-cyclopenten-1-one); (352) diftalone; (353) dimethylisopropylazulene; (354) 5,5-dimethyl-3-isopropyloxy-4-(4′-methylsulfonylphenyl)-2(5H)-furanone, (355) dimethyl sulfoxide; (356) DKA9 (4′-chloro-5-methoxy-3-biphenylylacetic acid); (357) DUP697 (selective COX-2 inhibitor); (358) EB382; (359) eicosatriynoic acid; (360) emorfazone; (361) enolicam; (362) ethyleneglycol salicylate; (363) F1044 (5-[5-(4-chlorophenyl-2-furanyl)]dihydro-2(3H)-furanone); (364) fenamates; (365) fenamole; (366) fenbuprofen; (367) fenclorac; (368) fenflumizole; (369) fenoprofen calcium; (370) floctafenine; (371) flunixin meglumine; (372) flurbiprofen axetil; (373) fosfosal; (374) furcloprofen; (375) glafenine; (376) glucametacin; (377) GP53633 (2-t-butyl-4(5)-phenyl-5(4)-(3-pyridyl)-imidazole); (378) 5(S)-HETE; (379) 5-HETE lactone; (380) ibuprofen aluminum; (381) ibuprofen piconol; (382) ibuproxam; (383) imidazole salicylate; (384) indometacin farnesil; (385) indomethacin sodium trihydrate; (386) indoxole (2,3-bis-(p-methoxyphenol)-indole); (387) intrazole; (388) ITC1 (2-methoxyethyl isothiocyanate); (389) ITF182 (imidazole 2-hydroxybenzoate); (390) JTE522 (4-(4-cyclohexyl-2 methyloxazol-5-yl)-2-fluorobenzensulphonamide); (391) KB1043 (2-(5-ethylpyridin-2-yl)benzimidazole); (392) KC8973 (4-butyl-2′-fluorobenzophenone); (393) ketophenylbutazone (kebuzone); (394) ketorolac tromethamine; (395) KME4; (396) LA2851 (2-4-diamino-7-methyl-pyrazolo (1,5-a) 1,3,5-triazine); (397) 5-lipoxygenase inhibitors; (398) lofemizole; (399) lonazolac calcium; (400) lotifazole; (401) lysine acetylsalicylate; (402) lysine clonixinate; (403) LU20884 (β-methyl[1,1′-biphenyl]-4-propanenitrile); (404) M7074 (6-chloro-4-oxyimino-1-phenyl-1,2,3,4-tetrahydroquinoline); (405) magnesium salicylate; (406) mefenamic acid aluminum; (407) mesalamine; (408) metamizole sothum; (409) metazamide; (410) metiazinic acid; (411) 6-methoxy-2 naphthylacetic acid; (412) MG18311 (4-((3-hydroxy-1H-indazol-1-yl)phenyl)acetic acid); (413) mixed PDE3/PDE4 inhibitors; (414) morniflumate (2-morpholin-4-ylethyl 2-{[3-(trifluoromethyl)phenyl]amino}nicotinate); (415) morpholine salicylate; (416) MR714 (2-(4-(2′,4′-difluorophenyl)-phenoxy)propionic acid); (417) MR897 (3-methyl-3-(4-acetylaminophenoxy)-2,4-dioxabenzocyclohexanone-1); (418) N-acetyl-5-aminosalicylic acid; (419) 1-naphthyl salicylate; (420) N-[2-(cyclohexyloxy)-4-nitrophenyl]methanesulfonamide; (421) neocinchophen; (422) nictindole; (423) nifenazone (N-(1,5-dimethyl-3-oxo-2-phenyl-2,3-dihydro-1H-pyrazol-4-yl)nicotinamide); (424) 2-(2-nitroxy)-butyl 2-acetoxybenzoate; (425) 2-(2-nitroxymethyl)phenyl 2-acetoxybenzoate; (426) NO164 (phenyl phosphonate derivative which is a partially selective antagonist of prostaglandin E₂); (427) NPPB (5-nitro-2(3-phenyl) propylamino-benzoic acid); (428) N-(2-pyridyl)-2-methyl-4-cinnamoyloxy-2H-1,2-benzothiazine-3-carboxamido 1,1-dioxide; (429) o-(acetoxyphenyl)hept-2-ynyl sulfide (APHS); (430) olsalazine oxaceprol; (431) olsalazine sodium; (432) oxametacin; (433) oxapadol; (434) oxicams; (435) oxyphenthatrazone; (436) paranylene; (437) peroxisal; (438) peroxisal citrate; (439) phenazone; (440) phenidone; (441) phenyl O-acetylsalicylate; (442) pifoxime; (443) piketoprofen; (444) pimeprofen; (445) piprofen; (446) piroxicam cinnamate; (447) proglumetacin maleate; (448) propyphenazone; (449) proquazone; (450) protizinic acid; (451) QZ16 (2-homopiperidino methyl-3-(o-tolyl)-4-(3H)-6-iodoquinazolone); (452) R830; (453) R-enantiomers of acrylacetic acids; (454) R-enantiomers of arylpropionic acids; (455) R-enantiomers of thiazinecarboxamides; (456) RS2131; (457) RS57067 (COX-2 inhibitor); (458) RU16029 (4-(2-methyl-3-(4-chlorobenzoyl)phenyl)butanoic acid); (459) salicylamide O-acetic acid; (460) SC560 (5-(4-chlorophenyl)-1-(4-methoxyphenyl)-3-(trifluoromethyl)-1H-pyrazole; a cyclooxygenase inhibitor); (461) SCR152; (462) sermetacin (N-[[1-(4-chlorobenzoyl)-5-methoxy-2-methyl-1H-indol-3-yl]acetyl]-L-serine); (463) sodium acetylsalicylate; (464) sodium thiosalicylate; (465) sulindac sulfide ((Z)-5-fluoro-2-methyl-1-[p-(methylthio)benzylidene]indene-3-acetic acid); (466) suxibutazone; (467) T614 (3-formylamino-7-methylsulfonylamino-6-phenoxy-4H-1-benzopyran-4-one); (468) TAI901 (4-benzoyl-1-indancarboxylic acid); (469) tesicam; (470) tetrydamine; (471) thromboxane inhibitors; (472) tiflamizole; (473) timegadine; (474) tinoridine hydrochloride; (475) tomoxiprol; (476) triethanolamine salicylate; (477) triflumidate; (478) trimethazone; (479) TVX960 (3′-hydroxy-2-[N-methyl-N-(1,1-dimethyl-2-phenethyl)amino]acetophenone); (480) TVX2706 (3-ethyl-1-(3-nitrophenyl)-2,4(1H,3H)-quinazolinedione); (481) TZI615 (6,11-dihydro-5-methyl-11-oxo-5H-dibenz[b,e]azepine-2-acetic acid); (482) U60257 (piriprost potassium salt); (483) ufenamate; (484) vedaprofen (4-cyclohexyl-alpha-methylnaphthalene-1-acetic acid); (485) WY23205 (3[4,5-di-p-chlorophenyloxazol-2-yl]propionic acid); (486) xenbucin; and (487) zileuton; and the salts, solvates, analogues, congeners, bioisosteres, hydrolysis products, metabolites, precursors, and prodrugs thereof.

Other NSAIDs that function as nitric oxide donors are disclosed in U.S. Pat. No. 6,297,260 to Bandarage et al., incorporated herein in its entirety by this reference.

The third agent includes serotonin or a serotonergic compound.

As used herein, the term “serotonin” includes, but is not limited to:

(1) serotonin sulfate; (2) serotonin creatinine sulfate complex; and (3) serotonin hydrochloride; as well as other salts and derivatives of serotonin as known in the art. As used herein, the term “serotonergic compound” means a compound that either acts as a serotonin agonist (i.e., can act in the same way as serotonin) or in another way increases the concentration, activity, or availability of serotonin.

Serotonergic compounds include, but are not limited to, the following classes of compounds: (1) serotonin transport inhibitors; (2) serotonin receptor 2C modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin agonists; (11) serotonin receptor 1A antagonists; and (12) serotonin metabolites. These categories are not exclusive, and many active serotonergic compounds suitable for inclusion in compositions of the present invention as the third agent can be considered to be in more than one of these categories; for example, such compounds can specifically interact with more than one class of serotonin receptor or more than one subclass of serotonin receptor within a single class.

Serotonin transport inhibitors include paroxetine, fluoxetine, fenfluramine, fluvoxamine, sertraline, imipramine, and compounds disclosed in PCT Patent Application Publication No. WO 03/00663, including phenyl-substituted piperazinylpyrimidines.

Serotonin receptor 2C modulators include BVT933, DPCA37215, IK264, (6-methyl-1,2,3,4,5,6-hexahydro-azepino[4,5-b]indole), WAY161503 (8,9-dichloro-2,3,4,4a-tetrahydro-1H-pyrazino[1,2-a]quinoxalin-5(6H)-one hydrochloride), R-1065, YM348 ((2S)-1-(7-ethyl-1H-furo[2,3-g]indazol-1-yl)propan-2-amine), and compounds disclosed in U.S. Pat. No. 3,914,250 and in PCT Patent Application Publication Nos. WO 01/66548, WO 02/10169, WO 02/36596, WO 02/40456, and WO 02/40457, WO 02/44152, WO 02/48124, WO 02/51844, and WO 03/033479, including 1,4-diazepino[6,5,4-jk]carbazoles, aza-indolyl derivatives, piperazine derivatives, cycloalkenyl[b][1,4]diazepino[6,7,1-hi]indoles and derivatives thereof, piperazinylpyrazine compounds, indoline derivatives, piperazine derivatives, and indole derivatives.

Serotonin reuptake inhibitors include arylpyrrolidine compounds, phenylpiperazine compounds, benzylpiperidine compounds, piperidine compounds, tricyclic gamma-carbolines, duloxetine compounds, pyrazinoquinoxaline compounds, pyridoindole compounds, piperidylindole compounds, milnacipran, citalopram, sertraline metabolite desmethylsertraline, norfluoxetine, citalopram metabolite desmethylcitalopram, escitalopram, d,l-fenfluramine, femoxetine, ifoxetine, cyanodothiepin, litoxetine, dapoxetine, nefazodone, cericlamine, trazodone, mirtazapine, fluoxetine, fluvoxamine, indalpine, indeloxazine, paroxetine, sertraline, sibutramine, zimeldine, trazodone hydrochloride, dexfenfluramine, and compounds disclosed in U.S. Pat. No. 6,365,633, PCT Patent Application Publication No. WO 01/27060, and PCT Patent Application Publicatio No. WO 01/162341, including (+)-N-[1-[1-(4-chlorophenyl)cyclobutyl]-3-methylbutyl]-N-methylamine, (−)-N-{1-[1-(4-chlorophenyl)cyclobutyl-3-methylbutyl}-N-methylamine, (+)-1-[1-(4-chlorophenyl)cyclobutyl]-3-methylbutylamine, (−)-1-O-(4-chlorophenyl)cyclobutyl]-3-methylbutylamine, (+)-N-{1-[1-(4-chlorophenyl)cyclobutyl]-3-methylbutyl}-N,N-dimethylamine, and (−)-N-{1-[1-(4-chlorophenyl)cyclobutyl]-3-methylbutyl}-N, -dimethylamine.

Serotonin and norepinephrine reuptake inhibitors include venlafaxine, venlafaxine metabolite O-desmethylvenlafaxine, clomipramine, and clomipramine metabolite desmethylclomipramine.

Serotonin dopamine antagonists include olanzapine and ziprasidone.

Monoamine reuptake inhibitors include amides.

Pyridazinone aldose reductase inhibitors include pyridazinone compounds.

Stimulants of serotonin receptors include ergoloid mesylate and pergolide mesylate.

Stimulants of serotonin synthesis include vitamin B1, vitamin B3, vitamin B6, biotin, S-adenosylmethionine, folic acid, folinic acid, derivatives of folic acid and folinic acid, ascorbic acid, magnesium, coenzyme Q10, and piracetam.

Serotonin agonists include fenfluramine and buspirone (a partial agonist for serotonin receptor 1A).

Serotonin receptor 1A antagonists include alprenolol, asenapine, BMY 7378 (8-(2-[4-(2-methoxyphenyl)-1-piperazinyl]ethyl)-8-azaspiro[4.5]decane-7,9-dione), cyanopindolol, iodocyanopindolol, lezcotozan, methiothepin, NAN-190 (1-(2-methoxyphenyl)-4-(4-phthalimidobutyl)piperazine), oxprenolol, pindolol, propranolol, robalzotan, S15535 (1-(2,3-dihydro-1,4-benzodioxin-8-yl)-4-(2,3-dihydro-1H-inden-2-yl)piperazine), spiperone, TFMPP, UH-301 ((S)-5-fluoro-8-hydroxy-2-(dipropylamino)tetralin), WAY-100,135 ((S)—N-tert-butyl-3-(4-(2-methoxyphenyl)-piperazin-1-yl)-2-phenylpropanamide), WAY-100,635 (N-[2-[4-(2-methoxyphenyl)-1-piperazinyl]ethyl]-N-(2-pyridyl)cyclohexanecarboxamide), and mefway.

Serotonin metabolites include, but are not limited to, 5-hydroxytryptophan, 5-methoxytryptamine, melatonin, or 5-HIAA (5-hydroxyindoleacetic acid). Preferably, the serotonin metabolite is present in the form of a creatinine sulfate complex, so that particularly preferred serotonin metabolites, in the form of a creatinine sulfate complex, include, but are not limited to, 5-hydroxytryptophan creatinine sulfate complex, 5-methoxytryptamine creatinine sulfate complex, melatonin creatinine sulfate complex, and 5-HIAA (5-hydroxyindoleacetic acid) creatinine sulfate complex. When the serotonin metabolite is included in the composition described above, it can be substantially free of impurities. For example, the serotonin metabolite can have a purity of at least about 80% (e.g., at least about 85%, at least about 90%, at least about 95%, or at least about 99%).

Many other serotonergic compounds and derivatives and metabolites thereof are known in the art and are included within the scope of the present application. Such serotonergic compounds and derivatives and metabolites thereof include: (1) serotonergic aminoalkylbenzadioxanes, such as those disclosed in U.S. Pat. No. 5,200,410; (2) serotonergic aminotetrahydrobenzindoles, such as those disclosed in U.S. Pat. No. 5,070,102; (3) serotonergic aminothiopyrans, such as those disclosed in U.S. Pat. No. 5,200,410; (4) serotonergic indolamines, such as those disclosed in U.S. Pat. No. 5,200,410; (5) serotonergic indolylalkylpiperidines, such as those disclosed in U.S. Pat. No. 5,200,410; (6) serotonergic monoamine oxidase inhibitors; (7) serotonergic tricyclic antidepressants; (7) serotonergic acetamide or carbamide derivatives, such as those disclosed in U.S. Pat. No. 6,756,393; (8) serotonergic 1-oxa-3,8-diaza-spiro[4.5]decan-2-one compounds such as those disclosed in U.S. Pat. No. 6,911,452; (9) serotonergic N-substituted piperidine derivatives, such as those disclosed in United States Patent Application Publication No. 2004/0106600; (10) serotonergic 2-pyrimidinyl-1-piperazines, such as those disclosed in U.S. Pat. No. 4,988,700; (11) serotonergic aryl-1-piperazines, such as those disclosed in U.S. Pat. No. 4,988,700; (12) serotonergic L-tryptophan derivatives and peptidyl derivatives of L-tryptophan, such as those disclosed in U.S. Pat. No. 6,579,899; (13) serotonin antagonists, such as those disclosed in United States Patent Application Publication No. 2001/0008896; and (14) serotonergic substituted dihydroergoline compounds, such as those disclosed in U.S. Pat. No. 4,798,834. Still other compounds are known in the art. Moreover, because of the multiplicity of classes and subclasses of serotonin receptors, some compounds may act as an agonist or partial agonist at one class or subclass of serotonin receptor, such as serotonin receptor 1A or 2A, and yet may act as an antagonist or inverse agonist at another class or subclass of serotonin receptor, such as serotonin receptor 2B, serotonin receptor 2C, serotonin receptor 6, or serotonin receptor 7.

Accordingly, suitable serotonergic compounds according to the present invention include, but are not limited to: (1) paroxetine; (2) fluoxetine; (3) fenfluramine; (4) fluvoxamine; (5) sertraline; (6) imipramine; (7) BVT933; (8) DPCA37215; (9) IK264; (10) PNU22394 (6-methyl-1,2,3,4,5,6-hexahydro-azepino[4,5-b]indole); (11) WAY161503 (8,9-dichloro-2,3,4,4a-tetrahydro-1H-pyrazino[1,2-a]quinoxalin-5(6H)-one hydrochloride); (12) R-1065; (13) YM348 ((2S)-1-(7-ethyl-1H-furo[2,3-g]indazol-1-yl)propan-2-amine); (14) milnacipran; (15) citalopram; (16) desmethylsertraline (a metabolite of sertraline); (17) norfluoxetine; (18) desmethylcitalopram (a metabolite of citalopram); (19) escitalopram; (20) femoxetine; (21) ifoxetine; (22) cyanodothiepin; (23) litoxetine; (24) dapoxetine; (25) nefazodone; (26) cericlamine; (27) trazodone; (28) mirtazapine; (29) indalpine; (30) indeloxazine; (31) sibutramine; (32) zimeldine; (33) (+)-N-[1-[1-(4-chlorophenyl)cyclobutyl]-3-methylbutyl]-N-methylamine; (34) (−)-N-{1-[1-(4-chlorophenyl)cyclobutyl-3-methylbutyl}-N-methylamine; (35) (−)-1-[1-(4-chlorophenyl)cyclobutyl]-3-methylbutylamine; (36) (+)-N-{1-[1-(4-chlorophenyl)cyclobutyl]-3-methylbutyl}-N; (37) (−)-N-{1-[1-(4-chlorophenyl)cyclobutyl]-3-methylbutyl}-N,N-dimethylamine)N-dimethylamine; (38) venlafaxine; (39) O-desmethylvenlafaxine (a metabolite of venlafaxine); (40) clomipramine; (41) desmethylclomipramine (a metabolite of clomipramine); (42) buspirone; (43) olanzapine; (44) ziprasidone; (45) ergoloid mesylates; (46) pergolide mesylate; (47) vitamin B1; (48) vitamin B3; (49) vitamin B6; (50) biotin; (51) S-adenosylmethionine; (52) folic acid; (53) folinic acid; (54) ascorbic acid; (55) magnesium; (56) coenzyme Q10; (57) piracetam; (58) (+)-2,5-dimethoxy-4-iodoamphetamine; (59) (+)-3,4-methylenedioxyamphetamine; (60) (+)-N-[2-[4-[2,3-dihydro-2-(hydroxymethyl)-1,4-benzodioxin-5-yl]1-piperazinyl]-4-fluorobenzamide hydrochloride; (61) (+)-norfenfluramine (a metabolite of fenfluramine); (62) (3β)-2,3-dihydrolysergene; (63) (3β)-2,3-dihydrolysergol; (64) (3β-2,3-dihydro-methyllysergate; (65) (3β, 5β, 8β)-9,10-didehydro-2,3-dihydro-6-methyl-8-(2-pyridylthiomethyl) ergoline; (66) (3β, 5β, 8β)-9,10-didehydro-2,3-dihydro-6-methyl-8-(methylthiomethyl) ergoline; (67) (3β, 5β, 8β)-9,10-didehydro-2,3-dihydro-6-methyl-8-(phenylthiomethyl) ergoline; (68) (3β, 5β, 8β)-9,10-didehydro-2,3-dihydro-8-methyl-6-propylergoline; (69) 1-(4-bromo-2,5-dimethoxyphenyl)-2-aminopropane; (70) 1-(m-trifluoromethylphenyl)-piperazine; (71) 2-(4-(4-(2-pyrimidinyl)1-piperazinyl-propyl)-1,2-benzoisothiazol-3-(2H)-one 1,1-dioxide hydrochloride; (72) 2-methylserotonin; (73) 3β, 5β, 8β)-9,10-didehydro-2,3-dihydro-6-methylergoline-8-acetonitrile; (74) zolmitriptan; (75) 3a,4,4a,6a,7,7a-hexahydro-2-[4-[4-(2-pyrimidinyl)-11-piperazinyl]butyl]-4,7-etheno-1H-cyclobutanoisoindole-1,3(2H)-dione dihydrochloride sesquihydrate; (76) 3-butyl-9,9-dimethyl-7-[4-[4-[2-methoxyphenyl) 1-piperazinyl]butyl]-3,7-diazabicyclo[3,2,1]nonane-2,4,6,8-tetraone; (77) 4,4-dimethyl-1-[4-[4-(2-pyrimidinyl)-1-piperazinyl]butyl]2,6-piperidinedione hydrochloride; (78) 5-hydroxy-L-tryptophan; (79) 5-methoxy-N,N-dimethyltryptamine; (80) 6-[[3-[4[o-methoxyphenyl]-1-piperazinyl]propyl]-amino]-1,3-dimethyluracil; (81) 8-[4-N-[4-(2-pyrimidinyl)-1-piperazinyl]butyl]-8-azaspiro[4.5]-decane-7,9-dione hydrochloride; (82) 8-hydroxy-2-(di-n-propylamino)tetralin (8-OH-DPAT); (83) alniditan; (84) almotriptan; (85) 2-aminotetralin; (86) bifeprunox; (87) gepirone; (88) BW723C86 (1-[5(2-thienylmethoxy)-1H-3-indolyl[propan-2-amine hydrochloride); (89) cisapride; (90) dihydroergotamine; (91) D-lysergic acid diethylamide; (92) donitriptan; (93) eletriptan; (94) frovatriptan; (95) tegaserod; (96) ipsapirone; (97) L694247 (2-[5-[3-(4-methylsulphonylamino)benzyl-1,2,4-oxadiazol-5-yl]-1H-indol-3-yl]ethanamine); (98) cinitapride; (99) lesopitron; (100) MCPP (m-chlorophenylpiperazine); (101) methysergide; (102) metoclopramide; (103) MK-212 (6-chloro-2-(1-piperazinyl)pyrazine hydrochloride); (104) mosapride; (105) N,N-dimethyl-5-methoxytryptamine; (106) N,N-dimethyltryptamine; (107) N-[4-[4-(2-pyrimidinyl)-1-piperazinyl]butylbicyclo[2.2.1]heptane-2,3-di-oxo-carboximide; (108) naratriptan; (109) norcisapride; (110) phentermine; (111) quipazine; (112) prucalopride; (113) rauwolscine; (114) repinotan; (115) rizatriptan; (116) sumatriptan; (117) tandospirone; (118) 1-methyl-4-phenyl-1,2,3,6-tetrahydropyridine; (119) tiaspirone; (120) trifluoromethylphenylpiperazine; (121) L-tryptophan; (122) xaliproden; (123) yohimbine; (124) zacopride; (125) zalospirone (126) mianserin; (127) setiptiline; (128) adatanserin; (129) altanserin; (130) benanserin; (131) blonanserin; (132) butanserin; (133) cinanserin; (134) eplivanserin; (135) flibanserin (136) glemanserin; (137) iferanserin; (138) ketanserin; (139) lidanserin; (140) pelanserin; (141) pruvanserin; (142) ritanserin; (143) seganserin; (144) tropanserin; (145) iloperidone; (146) sertindole; (147) EMR-62218; (148) asenapine; (149) zotepine; (150) ocaperidone; (151) APD125; (152) AVE8488; (153) pimavanserin; (154) isocarboxazid; (155) phenelzine; (156) tranylcypromine; (157) amitriptyline; (158) clomipramine; (159) N-(1-(1-methylethyl)piperidin-4-yl)-N-((4-methylphenyl)methyl)-4-methoxyphenylacetamide; (160) N-(1-(2,2-dimethylethyl)piperidin-4-yl)-N-((4-methylphenyl)methyl)-4-methoxyphenylacetamide; (161) N-(1-pentylpiperidin-4-yl)-N-((4-methylphenyl)methyl)-4-methoxyphenylacetamide; (162) N-(1-hexylpiperidin-4-yl)-N-((4-methylphenyl)methyl)-4-methoxyphenylacetamide, (163) N-(1-cyclohexylpiperidin-4-yl)-N-((4-methylphenyl)methyl)-4-methoxyphenylacetamide; (164) N-(1-cyclopentylpiperidin-4-yl)-N-((4-methylphenyl)methyl)-4-methoxyphenylacetamide; (165) N-(1-cyclobutylpiperidin-4-yl)-N-((4-methylphenyl)methyl)-4-methoxyphenylacetamide; (166) N-(1-cyclopropylpiperidin-4-yl)-N-((4-methylphenyl)methyl)-4-methoxyphenylacetamide; (167) N-(1-(cyclopentylmethyl)piperidin-4-yl)-N-((4-methylphenyl)methyl)-4-methoxyphenylacetamide; (168) N-(1-(cyclobutylmethyl)piperidin-4-yl)-N-((4-methylphenyl)methyl)-4-methoxyphenylacetamide; (169) N-(1-(cyclopropylmethyl)piperidin-4-yl)-N-((4-methylphenyl)methyl)-4-methoxyphenylacetamide; (170) N-(1-(2-hydroxyethyl)piperidin-4-yl)-N-((4-methylphenyl)methyl)-4-methoxyphenylacetamide; (171) N-(1-(3-hydroxypropyl)piperidin-4-yl)-N-((4-methylphenyl)methyl)-4-methoxyphenylacetamide; (172) N-((4-Methylphenyl)methyl)-N-(piperidin-4-yl)-N¹-phenylmethylcarbamide; (173) N-((4-Methylphenyl)methyl)-N-(1-(2-methylpropyl)piperidin-4-yl)-N′-phenylmethylcarbamide; (174) N-(1-((2-Bromophenyl)methyl)piperidin-4-yl)-N-((4-methylphenyl)methyl)-N′-phenylmethylcarbamide; (175) N-(1-((4-Hydroxy-3-methoxyphenyl)methyl)piperidin-4-yl)-N-((4-methylphenyl)methyl)-N′-phenylmethylcarbamide; (176) N-(1-((5-Ethylthien-2-yl)methyl)piperidin-4-yl)-N-((4-methylphenyl)methyl)-N′-phenylmethylcarbamide; (177) N-(1-(Imidazol-2-ylmethyl)piperidin-4-yl)-N-((4-methylphenyl)methyl)-N′-phenylmethylcarbamide; (178) N-(1-(Cyclohexylmethyl)piperidin-4-yl)-N-((4-methylphenyl)methyl)-N′-phenylmethylcarbamide; (179) N-(1-((4-Fluorophenyl)methyl)piperidin-4-yl)-N-((4-methylphenyl)methyl)-N′-phenylmethylcarbamide; (180) N-((4-Methylphenyl)methyl)-N-(piperidin-4-yl)-4-methoxyphenylacetamide; (181) N-((4-Methylphenyl)methyl)-N-(1-methylpiperidin-4-yl)-4-methoxyphenylacetamide; (182) N-(1-Ethylpiperidin-4-yl)-N-((4-methylphenyl)methyl)-4-methoxyphenylacetamide; (183) N-((4-Methylphenyl)methyl)-N-(1-propylpiperidin-4-yl)-4-methoxyphenylacetamide; (184) N-(1-Butylpiperidin-4-yl)-N-((4-methylphenyl)methyl)-4-methoxyphenylacetamide; (185) N-(1-(3,3-Dimethylbutyl)piperidin-4-yl)-N-((4-methylphenyl)methyl)-4-methoxyphenylacetamide; (186) N-(1-(Cyclohexylmethyl)piperidin-4-yl)-N-((4-methylphenyl)methyl)-4-methoxyphenylacetamide; (187) N-((4-Methylphenyl)methyl)-N-(1-(2-methylpropyl)piperidin-4-yl)-4-methoxyphenylacetamide; (188) N-((4-Methylphenyl)methyl)-N-(1-((4-methylphenyl)methyl)piperidin-4-yl)-4-methoxyphenylacetamide; (189) N-((1-(4-Hydroxyphenyl)methyl)piperidin-4-yl)-N-((4-methylphenyl)methyl)-4-methoxyphenylacetamide; (190) N-(1-((2-Hydroxyphenyl)methyl)piperidin-4-yl)-N-((4-methylphenyl)methyl)-4-methoxyphenylacetamide; (191) N-(3-Phenylpropyl)-N-(piperidin-4-yl)-4-methoxyphenylacetamide; (192) N-(2-Phenylethyl)-N-(piperidin-4-yl)-4-methoxyphenylacetamide; (193) N-((2-Methoxyphenyl)methyl)-N-(piperidin-4-yl)-4-methoxyphenylacetamide; (194) N-((2-Chlorophenyl)methyl)-N-(piperidin-4-yl)-4-methoxyphenylacetamide; (195) N-((3,4-Di-methoxyphenyl)methyl)-N-(piperidin-4-yl)-4-methoxyphenylacetamide; (196) N-((4-Fluorophenyl)methyl)-N-(piperidin-4-yl)-4-methoxyphenylacetamide; (197) N-((2,4-Di-chlorophenyl)methyl)-N-(piperidin-4-yl)-4-methoxyphenylacetamide; (198) N-((3-Methylphenyl)methyl)-N-(piperidin-4-yl)-4-methoxyphenylacetamide; (199) N-((3-Bromophenyl)methyl)-N-(piperidin-4-yl)-4-methoxyphenylacetamide; (200) N-(1-(Phenylmethyl)piperidin-4-yl)-N-(3-phenyl-2-propen-1-yl)-4-methoxyphenylacetamide; (201) N-((4-Methylphenyl)methyl)-N-(1-piperidin-4-yl)-phenylacetamide; (202) N-((4-Methylphenyl)methyl)-N-(1-piperidin-4-yl)-3-phenylpropionamide; (203) N-((4-Methylphenyl)methyl)-N-(1-piperidin-4-yl)-(phenylthio)acetamide; (204) N-((4-Methylphenyl)methyl)-N-(1-piperidin-4-yl)-phenoxyacetamide; (205) N-((4-Methylphenyl)methyl)-N-(1-piperidin-4-yl)-(4-chlorophenoxy)acetamide; (206) N-((4-Methylphenyl)methyl)-N-(1-piperidin-4-yl)-3-methoxyphenylacetamide; (207) N-((4-Methylphenyl)methyl)-N-(1-piperidin-4-yl)-4-fluorophenylacetamide; (208) N-((4-Methylphenyl)methyl)-N-(1-piperidin-4-yl)-2,5-di-methoxyphenylacetamide; (209) N-((4-Methylphenyl)methyl)-N-(1-piperidin-4-yl)-4-chlorophenylacetamide; (210) N-((4-Methylphenyl)methyl)-N-(1-(phenylmethyl)pyrrolidin-3-yl)-N′-phenylmethylcarbamide; (211) N-((4-Methylphenyl)methyl)-N-(1-(phenylmethyl)pyrrolidin-3-yl)-4-methoxyphenylacetamide; (212) 2-(4-methoxyphenyl)-N-(4-methylbenzyl)-N-(piperidin-4-yl)acetamide; (213) 2-(4-methoxyphenyl)-N-(4-methylbenzyl)-N-(1-methylpiperidin-4-yl)acetamide; (214) 244-methoxyphenyl)-N-(4-methylbenzyl)-N-(1-ethylpiperidin-4-yl)acetamide; (215) 2-(4-methoxyphenyl)-N-(4-chlorbenzyl)-N-(1-ethylpiperidin-4-yl)acetamide; (216) 2-(4-methoxyphenyl)-N-(4-chlorbenzyl)-N-(1-isopropylpiperidin-4-yl)acetamide; (217) 2-(4-methoxyphenyl)-N-(4-chlorobenzyl)-N-(piperidin-4-yl)acetamide; (218) 2-(4-methoxyphenyl)-N-(4-chlorobenzyl)-N-(1-cyclopentylpiperidin-4-yl)acetamide; (219) 2-(4-methoxyphenyl)-N-(4-chlorbenzyl)-N-(1-isopropylpiperidin-4-yl)acetamide, (220) 2-(phenyl)-N-(4-trifluoromethylbenzyl)-N-(1-methylpiperidin-4-yl)acetamide; (221) 2-(4-fluorophenyl)-N-(4-trifluoromethylbenzyl)-N-(1-methylpiperidin-4-yl)acetamide; (222) 2-(4-Methoxyphenyl)-N-(4-trifluoromethylbenzyl)-N-(1-methylpiperidin-4-yl)acetamide; (223) 2-(4-Trifluoromethylphenyl)-N-(4-trifluoromethylbenzyl)-N-(1-methylpiperidin-4-yl)acetamide; (224) 2-(4-Fluorophenyl)-N-(4-fluorobenzyl)-N-(1-methylpiperidin-4-yl)acetamide; (225) 2-(4-Methoxyphenyl)-N-(4-fluorobenzyl)-N-(1-methylpiperidin-4-yl)acetamide; (226) 2-(phenyl)-N-(4-fluorobenzyl)-N-(1-methylpiperidin-4-yl)acetamide; (227) 2-(4-Trifluoromethylphenyl)-N-(4-fluorobenzyl)-N-(1-methylpiperidin-4-yl)acetamide; (228) 2-(4-trifluoromethylphenyl)-N-[4-(methoxycarbonyl)benzyl]-N-(1-methylpiperidin-4-yl)acetamide; (229) 2-Phenyl-N-[4-(methoxycarbonyl)benzyl]-N-(1-methylpiperidin-4-yl)acetamide; (230) 2-(4-Chlorophenyl)-N-[4-(methoxycarbonyl)benzyl]-N-(1-methylpiperidin-4-yl)acetamide; (231) 2-(4-Methoxyphenyl)-N-[4-(methoxycarbonyl)benzyl]-N-(1-methylpiperidin-4-yl)acetamide; (232) 2-(4-trifluoromethylphenyl)-N-[4-(methoxycarbonyl)benzyl]-N-(1-methylpiperidin-4-yl)acetamide; (233) 2-Phenyl-N-[4-(methoxycarbonyl)benzyl]-N-(1-methylpiperidin-4-yl)acetamide, (234) 2-(4-Chlorophenyl)-N-[4-(methoxycarbonyl)benzyl]-N-(1-methylpiperidin-4-yl)acetamide; (235) 2-(4-Methoxyphenyl)-N-[4-(methoxycarbonyl)benzyl]-N-(1-methylpiperidin-4-yl)acetamide; (236) 2-(4-methoxyphenyl)-N-(4-methylbenzyl)-N-[1-(4-chloromethyl-2-thiazol yl methyl)piperidin-4-yl]acetamide; (237) 2-(4 methoxyphenyl)-N-(4-methylbenzyl)-N-{1-[3-(1,3-dihydro-2H-benzimidazol-2-on-1-yl)propyl]piperidin-4-yl}acetamide; (238) 2-(4-methoxyphenyl)-N-(2-4(fluorophenyl)ethyl)-N-(1-methylpiperidin-4-yl)acetamide; (239) 2-(4-methoxyphenyl)-N-[2-(2,5-dimethoxyphenyl)ethyl]-N-(1-methylpiperidin-4-yl)acetamide; (240) 2-(4-methoxyphenyl)-N-[2-(2,4-dichlorophenyl)ethyl]-N-(1-methylpiperidin-4-yl)acetamide; (241) 2-(4-methoxyphenyl)-N-[2-(3-chlorophenyl)ethyl]-N-(1-methylpiperidin-4-yl)acetamide; (242) 2-(4-methoxyphenyl)-N-[2-(4-methoxyphenyl)ethyl]-N-(1-methylpiperidin-4-yl)acetamide; (243) 2-(4-methoxyphenyl)-N-[2-(3-fluorophenyl)ethyl]-N-(1-methylpiperidin-4-yl)acetamide; (244) 2-(4-ethoxyphenyl)-N-[2-(4-fluorophenethyl]-N-(1-methylpiperidin-4-yl)acetamide; (245) 2-(4-ethoxyphenyl)-N-(4-fluorobenzyl)-N-(1-methylpiperidin-4-yl)acetamide; (246) 2-(4-methoxyphenyl)-N-(4-methylbenzyl)-N-{1-[2-(2-hydroxyethoxy)ethyl]piperidin-4-yl}acetamide; (247) 2-(4-methoxyphenyl)-N-(4-methylbenzyl)-N-[1-((2-chloro-5-thienyl)methyl)piperidin-4-yl]acetamide; (248) 2-(4-methoxyphenyl)-N-(4-methylbenzyl)-N-[1-(2-(imidazolidinon-1-yl)ethyl)piperidin-4-yl]acetamide; (249) 2-(4-methoxyphenyl)-N-(4-methylbenzyl)-N-{1-[2-(2,4(1H,3H)quinazolinedion-3-yl)ethyl]piperidin-4-yl}acetamide; (250) 2-(4-methoxyphenyl)-N-(4-methylbenzyl)-N-{1-[2-(1,3-dioxolan-2-yl)ethyl]piperidin-4-yl}acetamide; (251) 2-(4-methoxyphenyl)-N-(4-methylbenzyl)-N-{1-[2-(3-indolyl)ethyl]piperidin-4-yl}acetamide; (252) 2-(4-methoxyphenyl)-N-(4-methylbenzyl)-N-{1-[3-(1,2,4-triazol-1-yl)propyl]piperidin-4-yl}acetamide, (253) 2-(4-methoxyphenyl)-N-(4-methylbenzyl)-N-[1-(5-benzofurazanylmethyl)piperidin-4-yl]acetamide; (254) 2-(4-methoxyphenyl)-N-(4-methylbenzyl)-N-[1-(5-chlorobenzo[b]thien-3-ylmethyl)piperidin-4-yl]acetamide; (255) 2-(4-methoxyphenyl)-N-(4-methylbenzyl)-N-[1-(5-phenyl-1,2,4-oxadiazol-3-ylmethyl)piperidin-4-yl]acetamide; (256) 2-(4-Chlorophenyl)-N-(4-methylbenzyl)-N-(1-isopropylpiperidin-4-yl)-acetamide; (257) 2-(4-Chlorophenyl)-N-(4-methylbenzyl)-N-(1-ethylpiperidin-4-yl)-acetamide; (258) 2-Phenyl-N-(4-methylbenzyl)-N-(1-methylpiperidin-4-yl)-acetamide; (259) 2-(4-Chlorophenyl)-N-(4-methylbenzyl)-N-(1-methylpiperidin-4-yl)-acetamide; (260) 2-(4-Chlorophenyl)-N-(4-methylbenzyl)-N-(1-cyclopentylpiperidin-4-yl)-acetamide; (261) 2-(4-Fluorophenyl)-N-(4-methylbenzyl)-N-(1-methylpiperidin-4-yl)-acetamide; (262) 2-(4-Chlorophenyl)-N-(4-methylbenzyl)-N-(1-(2-hydroxyethyl)-piperidin-4-yl)-acetamide; (263) 2-(4-Chlorophenyl)-N-(4-methylbenzyl)-N-(1-cyclobutylpiperidin-4-yl)-acetamide; (264) 2-(4-Methoxyphenyl)-N-(4-methylbenzyl)-N-(1-cyclobutylpiperidin-4-yl)-acetamide; (265) 2-(4-Methoxyphenyl)-N-(4-methylbenzyl)-N-(tropin-4-yl)-acetamide; (266) N-(4-Methylbenzyl)-N-(1-methylpiperidin-4-yl)-N′-benzyl-carbamide; (267) N-(4-Methylbenzyl)-N-(1-methylpiperidin-4-yl)-N′-phenyl-carbamide; (268) N-Phenethyl-N-(1-methylpiperidin-4-yl)-N′-benzyl-carbamide; (269) 2-Phenyl-N-(4-methoxybenzyl)-N-(1-methylpiperidin-4-yl)-acetamide; (270) 2-(4-Trifluoromethylphenyl)-N-(4-methoxybenzyl)-N-(1-methylpiperidin-4-yl)-acetamide (271) 2-(4-Fluorophenyl)-N-(4-methoxybenzyl)-N-(1-methylpiperidin-4-yl)-acetamide; (272) 2-(4-Methoxyphenyl)-N-(4-methoxybenzyl)-N-(1-methylpiperidin-4-yl)-acetamide; (273) 2-(4-Methylphenyl)-N-(4-chlorobenzyl)-N-(1-methylpiperidin-4-yl)-acetamide; (274) 2-(4-Hydroxyphenyl)-N-(4-methylbenzyl)-N-(1-methylpiperidin-4-yl)-acetamide; (275) N-Phenethyl-N-(1-methylpiperidin-4-yl)-N′-phenyl-carbamide; (276) N-(3-Phenylpropyl)-N-(1-methylpiperidin-4-yl)-N′-benzyl-carbamide; (277) N-(3-Phenylpropyl)-N-(1-methylpiperidin-4-yl)-W-phenyl-carbamide; (278) 2-(4-Methoxyphenyl)-2,2-ethylene-N-(4-methylbenzyl)-N-(1-methylpiperidin-4-yl)acetamide; (279) 2-(4-Methoxyphenyl)-N-alpha-methylbenzyl-N-(1-methylpiperidin-4-yl)acetamide; (280) 2-(4-Methoxyphenyl)-N-(4-methylbenzyl)-N-(3-tropen-4-yl)acetamide; (281) 2-Phenyl-2-ethyl-N-(4-methylbenzyl)-N-(1-methylpiperidin-4-yl)acetamide; (282) N-Phenethyl-N-(4-methylbenzyl)-N-(1-methylpiperidin-4-yl)-amine; (283) 2-(4-Methoxyphenyl)-N-(1-indanyl)-N-(1-methylpiperidin-4-yl)acetamide; (284) N-(4-Methylbenzyl)-N-(1-methylpiperidin-4-yl)-N′-(4-methoxybenzyl)-carbamide; (285) 2-(3,4-dimethoxyphenyl)-N-(4-methylbenzyl)-N-(1-methylpiperidin-4-yl)acetamide; (286) 2-(3,4-Methylenedioxyphenyl)-N-(4-methylbenzyl)-N-(1-methylpiperidin-4-yl)acetamide; (287) 2-(4-Methoxyphenyl)-N-(4-methylbenzyl)-N-(1-t-butylpiperidin-4-yl)-acetamide; (288) N-(4-Methylbenzyl)-N-(1-methylpiperidin-4-yl)-N′-phenethyl-carbamide; (289) N-Phenethyl-N-(1-methylpiperidin-4-yl)-N′-phenethyl-carbamide; (290) N-(4-Methylbenzyl)-N-(1-t-butylpiperidin-4-yl)-N′-(4-methoxybenzyl)-carbamide; (291) 2-(4-Ethoxyphenyl)-N-(4-methylbenzyl)-N-(1-methylpiperidin-4-yl)acetamide; (292) 2-(4-Butoxyphenyl)-N-(4-methylbenzyl)-N-(1-methylpiperidin-4-yl)acetamide; (293) 2-(4-i-Propoxyphenyl)-N-(4-methylbenzyl)-N-(1-methylpiperidin-4-yl)acetamide; (294) 2-(4-t-Butoxyphenyl)-N-(4-methylbenzyl)-N-(1-methylpiperidin-4-yl)acetamide; (295) 2-(4-Butoxyphenyl)-N-(4-fluorobenzyl)-N-(1-methylpiperidin-4-yl)acetamide; (296) 2-(4-Propoxyphenyl)-N-(4-fluorobenzyl)-N-(1-methylpiperidin-4-yl)acetamide; (297) 2-(4-i-Propoxyphenyl)-N-(4-fluorobenzyl)-N-(1-methylpiperidin-4-yl)acetamide, (298) 2-(4-t-Butoxyphenyl)-N-(4-fluorobenzyl)-N-(1-methylpiperidin-4-yl)acetamide; (299) 4-(4-Fluorobenzyl)-3-(4-methoxybenzyl)-8-methyl-1-oxa-3,8-diaza-spiro[4.5]decan-2-one; (300) 3-(4-Ethoxybenzyl)-4-(4-fluorobenzyl)-8-methyl-1-oxa-3,8-diaza-spiro[4.5]decan-2-one; (301) 4-(4-Fluorobenzyl)-8-methyl-3-(4-propoxybenzyl)-1-oxa-3,8-diaza-spiro[4.5]decan-2-one; (302) 3-(4-Cyclopropylmethoxybenzyl)-4-(4-fluorobenzyl)-8-methyl-1-oxa-3,8-diaza-spiro[4.5]decan-2-one; (303) 4-(4-Fluorobenzyl)-3-(4-isopropoxybenzyl)-8-methyl-1-oxa-3,8-diaza-spiro[4.5]decan-2-one; (304) 3-(4-Butoxybenzyl)-4-(4-fluorobenzyl)-8-methyl-1-oxa-3,8-diaza-spiro[4.5]decan-2-one; (305) 4-(4-Fluorobenzyl)-3-(4-isobutoxybenzyl)-8-methyl-1-oxa-3,8-diaza-spiro[4.5]decan-2-one, (306) 3-(4-Difluoromethoxybenzyl)-4-(4-fluorobenzyl)-8-methyl-1-oxa-3,8-diaza-spiro[4.5]decan-2-one; (307) 4-(4-Fluorobenzyl)-8-methyl-3-(4-trifluoromethoxybenzyl)-1-oxa-3,8-diaza-spiro[4.5]decan-2-one; (308) 4-(4-Fluorobenzyl)-8-methyl-3-(4-pentoxybenzyl)-1-oxa-3,8-diaza-spiro[4.5]decan-2-one; (309) 8-Ethyl-4-(4-fluorobenzyl)-3-(4-isobutoxybenzyl)-1-oxa-3,8-diaza-spiro[4.5]decan-2-one; (310) 4-(4-Fluorobenzyl)-3-(4-isobutoxybenzyl)-8-isopropyl-1-oxa-3,8-diaza-spiro[4.5]decan-2-one; (311) 8-Cyclopropylmethyl-4-(4-fluorobenzyl)-3-(4-isobutoxybenzyl)-1-oxa-3,8-diaza-spiro[4.5]decan-2-one; (312) 8-Cyclohexylmethyl-4-(4-fluorobenzyl)-3-(4-isobutoxybenzyl)-1-oxa-3,8-diaza-spiro[4.5]decan-2-one; (313) 8-Cyclopentyl-4-(4-fluorobenzyl)-3-(4-isobutoxybenzyl)-1-oxa-3,8-diaza-spiro[4.5]decan-2-one, (314) 4-(4-Fluorobenzyl)-3-(4-isobutoxybenzyl)-8-(3-morpholin-4-yl-propyl)-1-oxa-3,8-diaza-spiro[4.5]decan-2-one; (315) 8-(2-[1,3]Dioxolan-2-yl-ethyl)-4-(4-fluorobenzyl)-3-(4-isobutoxybenzyl)-1-oxa-3,8-diaza-spiro[4.5]decan-2-one; (316) 4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one; (317) 4-(4-Fluorobenzyl)-3-(4-isobutoxybenzyl)-8-[3-(2-oxo-2,3-dihydro-benzoimidazol-1-yl)-propyl]-1-oxa-3,8-diaza-spiro[4.5]decan-2-one; (318) 4-(4-Fluorobenzyl)-3-(4-isobutoxybenzyl)-8-(2-methyl-thiazol-4-yl-methyl)-1-oxa-3,8-diaza-spiro[4.5]decan-2-one; (319) 4-(4-Chlorobenzyl)-3-(4-isobutoxybenzyl)-8-methyl-1-oxa-3,8-diaza-spiro[4.5]decan-2-one; (320) 8-Ethyl-4-(4-chlorobenzyl)-3-(4-isobutoxybenzyl)-1-oxa-3,8-diaza-spiro[4.5]decan-2-one; (321) 4-(4-Chlorobenzyl)-3-(4-isobutoxybenzyl)-8-isopropyl-1-oxa-3,8-diaza-spiro[4.5]decan-2-one; (322) 8-Cyclopropylmethyl-4-(4-chlorobenzyl)-3-(4-isobutoxybenzyl)-1-oxa-3,8-diaza-spiro[4.5]decan-2-one; (323) 8-Cyclohexylmethyl-4-(4-chlorobenzyl)-3-(4-isobutoxybenzyl)-1-oxa-3,8-diaza-spiro[4.5]decan-2-one; (324) 8-(2-[1,3]Dioxolan-2-yl-ethyl)-4-(4-chlorobenzyl)-3-(4-isobutoxybenzyl)-1-oxa-3,8-diaza-spiro[4.5]decan-2-one; (325) 4-(4-Chlorobenzyl)-3-(4-isobutoxybenzyl)-8-[2-(2-oxo-imidazolidin-1-yl)-ethyl]-1-oxa-3,8-diaza-spiro[4.5]decan-2-one; (326) 3-(4-Difluoromethoxybenzyl)-4-(4-fluorobenzyl)-8-methyl-1-oxa-3,8-diaza-spiro[4.5]decan-2-one; (327) 3-(4-Difluoromethoxybenzyl)-8-ethyl-4-(4-fluorobenzyl)-1-oxa-3,8-diaza-spiro[4.5]decan-2-one; (328) 3-(4-Difluoromethoxybenzyl)-4-(4-fluorobenzyl)-8-isopropyl-1-oxa-3,8-diaza-spiro[4.5]decan-2-one; (329) 8-Cyclopropylmethyl-3-(4-difluoromethoxybenzyl)-4-(4-fluorobenzyl)-1-oxa-3,8-diaza-spiro[4.5]decan-2-one; (330) 8-Cyclohexylmethyl-3-(4-difluoromethoxybenzyl)-4-(4-fluorobenzyl)-1-oxa-3,8-diaza-spiro[4.5]decan-2-one; (331) 3-(4-Difluoromethoxybenzyl)-8-(2-[1,3]dioxolan-2-yl-ethyl)-4-(4-fluorobenzyl)-1-oxa-3,8-diaza-spiro[4.5]decan-2-one; (332) 3-(4-Difluoromethoxybenzyl)-4-(4-fluorobenzyl)-8-[2-(2-oxo-imidazolidin-1-yl)-ethyl]-1-oxa-3,8-diaza-spiro[4.5]decan-2-one; (333) 8-Ethyl-4-(4-fluorobenzyl)-3-(4-trifluoromethoxybenzyl)-1-oxa-3,8-diaza-spiro[4.5]decan-2-one; (334) 4-(4-Fluorobenzyl)-8-isopropyl-3-(4-trifluoromethoxybenzyl)-1-oxa-3,8-diaza-spiro[4,5]decan-2-one; (335) 8-Cyclopropylmethyl-4-(4-fluorobenzyl)-3-(4-trifluoromethoxybenzyl)-1-oxa-3,8-diaza-spiro[4.5]decan-2-one; (336) 8-Cyclohexylmethyl-4-(4-fluorobenzyl)-3-(4-trifluoromethoxybenzyl)-1-oxa-3,8-diaza-spiro[4.5]decan-2-one; (337) 8-Cyclopentyl-4-(4-fluorobenzyl)-3-(4-trifluoromethoxybenzyl)-1-oxa-3,8-diaza-spiro[4.5]decan-2-one; (338) 8-(2-[1,3]Dioxolan-2-yl-ethyl)-4-(4-fluorobenzyl)-3-(4-trifluoromethoxybenzyl)-1-oxa-3,8-diaza-spiro[4.5]decan-2-one; (339) 4-(4-Fluorobenzyl)-8-[2-(2-oxo-imidazolidin-1-yl)-ethyl]-3-(4-trifluoromethoxybenzyl)-1-oxa-3,8-diaza-spiro[4.5]decan-2-one; (340) 8-Ethyl-4-(4-fluorobenzyl)-3-(4-propoxybenzyl)-1-oxa-3,8-diaza-spiro[4.5]decan-2-one; (341) 4-(4-Fluorobenzyl)-8-isopropyl-3-(4-propoxybenzyl)-1-oxa-3,8-diaza-spiro[4.5]decan-2-one; (342) 8-Cyclopropylmethyl-4-(4-fluorobenzyl)-3-(4-propoxybenzyl)-1-oxa-3,8-diaza-spiro[4.5]decan-2-one; (343) 8-Cyclohexylmethyl-4-(4-fluorobenzyl)-3-(4-propoxybenzyl)-1-oxa-3,8-diaza-spiro[4.5]decan-2-one; (344) 8-Cyclopentyl-4-(4-fluorobenzyl)-3-(4-propoxybenzyl)-1-oxa-3,8-diaza-spiro[4.5]decan-2-one; (345) 8-(2-[1,3]Dioxolan-2-yl-ethyl)-4-(4-fluorobenzyl)-3-(4-propoxybenzyl)-1-oxa-3,8-diaza-spiro[4.5]decan-2-one; (346) 4-(4-Fluorobenzyl)-8-[2-(2-oxo-imidazolidin-1-yl)-ethyl]-3-(4-propoxybenzyl)-1-oxa-3,8-diaza-spiro[4,5]decan-2-one; (347) 3-(4-Cyclopropyl methoxybenzyl)-8-ethyl-4-(4-fluorobenzyl)-1-oxa-3,8-diaza-spiro[4.5]decan-2-one; (348) 3-(4-Cyclopropylmethoxybenzyl)-4-(4-fluorobenzyl)-8-isopropyl-1-oxa-3,8-diaza-spiro[4.5]decan-2-one; (349) 3-(4-Cyclopropylmethoxybenzyl)-8-cyclopropylmethyl-4-(4-fluorobenzyl)-1-oxa-3,8-diaza-spiro[4.5]decan-2-one; (350) 3-(4-Cyclopropylmethoxybenzyl)-8-(2-[1,3]dioxolan-2-yl-ethyl)-4-(4-fluorobenzyl)-1-oxa-3,8-diaza-spiro[4.5]decan-2-one; (351) 3-(4-Cyclopropylmethoxybenzyl)-4-(4-fluorobenzyl)-8-[2-(2-oxo-imidazolidin-1-yl)-ethyl]-1-oxa-3,8-diaza-spiro[4.5]decan-2-one; (352) 8-(2-[1.3]-Dioxan-2-yl-ethyl)-4-(4-fluorobenzyl)-3-(4-isobutoxybenzyl)-1-oxa-3,8-diaza-spiro[4.5]decane-3-one; (353) 4-(4-Fluorobenzyl)-3-(4-isobutoxybenzyl)-8-{3-[(S)-4-isopropyl-2-oxo-oxazolidin-3-yl]-propyl}-1-oxa-3,8-diaza-spiro[4.5]decane-3-one; (354) N-{1-[2-(1,3-Dioxolan-2-yl)ethyl]piperidin-4-yl}-N-(4-fluorobenzyl)-N′-(4-isobutoxybenzyl)carbamide hydrochloride; (355) N-{1-[2-(1,3-Dioxan-2-yl)ethyl]-piperidin-4-yl}-N-(4-fluorobenzyl)-2-[4-(2-hydroxy-2-methylpropoxy)phenyl]-acetamide tartrate; (356) N-(4-Fluorobenyzl)-N-(piperidin-4-yl)-2-(4-isobutoxyphenyl)acetamide; (357) N-{1-[3-(3,5-Dimethylpiperidin-1-yl)propyl]piperidin-4-yl-}N-(4-fluorobenzyl)-2-(4-isobutoxyphenyl)acetamide dihydrochloride; (358) 1-[3-(4-{(4-Fluorobenzyl)-[2-(4 isobutoxyphenyl)acetyl]amino}piperidin-1-yl)propyl]piperidine-4-carboxylic acid methyl ester dihydrochloride; (359) N-(4-Fluorobenzyl)-2-(4-isobutoxyphenyl)-N-{1-[2-(1-methylpyrrolidin-2-yl-)ethyl]piperidin-4-yl}acetamide dioxalate; (360) N-{1-[3-(2,6-Dimethylmorpholin-4-yl)propyl]piperidin-4-yl}-N-(4-fluorobenzyl)-2-(4-isobutoxyphenyl)acetamide dioxalate; (361) N-(4-Fluorobenzyl)-N-{1-[3-(3-hydroxypiperidin-1-yl)propyl]piperidin-4-yl}-2-(4-isobutoxyphenyl)acetamide dioxalate; (362) N-(4-Fluorobenzyl)-2-(4-isobutoxyphenyl)-N-{1-[3-(2-methylpiperidin-1-yl)-propyl]piperidin-4-yl}acetamide dioxalate; (363) N-(4-Fluorobenzyl-2-(4-isobutoxyphenyl)-N-[1-(3-pyrrolidin-1-yl-propyl)piperidin-4-yl]acetamide dioxalate; (364) N-{1-[3-(2,5-Dimethylpyrrolidin-1-yl)propyl]piperidin-4-yl}-N-(4-fluorobenzyl)-2-(4-isobutoxyphenyl)acetamide dioxalate; (365) N-(4-Fluorobenzyl)-N-{1-[3-(3-hydroxymethylpiperidin-1-yl)propyl]piperidin-4-yl}-2-(4-isobutoxyphenyl)acetamide dioxalate; (366) N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide oxalate; (367) N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide oxalate; (368) N-[2-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide oxalate; (369) N-(4-Fluorobenzyl)-N-{1-[3-(4-(S)-isopropyl-2-oxo-oxazolidin-3-yl)propyl]piperidin-4-yl}-2-(4-propoxyphenyl)acetamide oxalate; (370) N-{1-[2-(1,3-Dioxan-2-yl)ethyl]piperidin-4-yl}-N-(4-fluorobenzyl)-2-(4-isobutoxyphenyl)acetamide oxalate; (371) N-{1-[2-(1,3-Dioxan-2-yl)ethyl]piperidin-4-yl}-N-[2-(4-fluorophenyl)ethyl]-2-(4-isobutoxyphenyl)acetamide oxalate; (372) N-{1-[2-(1,3-Dioxan-2-yl)ethyl]piperidin-4-yl}-N-[2-(4-fluorophenyl)ethyl]-2-(4-propoxyphenyl)acetamide oxalate; (373) N-{1-[2-(1,3-Dioxan-2-yl-)ethyl]piperidin-4-yl}-N-(4-fluorobenzyl)-2-(4-propoxyphenyl)acetamide tartrate; (374) N-{1-[2-(1,3-Dioxan-2-yl)ethyl]piperidin-4-yl}-N-(4-fluorobenzyl)-N′-(4-isobutoxybenzyl)carbamide tartrate; (375) N-{1-[2-(1,3-Dioxan-2-yl)ethyl]piperidin-4-yl}-N-(4-fluorobenzyl)-2-(4-fluorophenyl)acetamide tartrate; (376) N-{1-[2-(1,3-Dioxan-2-yl)ethyl]piperidin-4-yl}-N-(4-fluorobenzyl)-2-p-tolylacetamide tartrate; (377) 2-Benzofuran-5-yl-N-{1-[2-(1,3-dioxan-2-yl)ethyl]piperidin-4-yl}-N-(4-fluorobenzyl)acetamide tartrate; (378) 2-(2,3-Dihydrobenzofuran-5-yl)-N-{1-[2-(1,3-dioxan-2-yl)ethyl]piperidin-4-yl}-N-(4-fluorobenzyl)acetamide tartrate; (379) N-{1-[2-(2,2-Dimethyl-1,3-dioxolan-4-yl)ethyl]piperidin-4-yl}-N-(4-fluorobenzyl)-2-(4-isobutoxyphenyl)acetamide tartrate; (380) N-{1-[2-(1,3-Dioxan-4-yl)ethyl]piperidin-4-yl}-N-(4-fluorobenzyl)amine; (381) N{-1-[2-(1,3-Dioxan-4-yl)ethyl]piperidin-4-yl}-N-(4-fluorobenzyl)-2-(4-isobutoxyphenyl)acetamide tartrate; (382) N-{1-[2-(1,3-Dioxan-4-yl)ethyl]piperidin-4-yl}-N(4-fluorobenzyl)-2-(4-trifluoromethylphenyl)acetamide tartrate; (383) 2-(4-Cyanophenyl)-N-{1-[2-(1,3-dioxan-4-yl)ethyl]piperidin-4-yl}-N-(4-fluorobenzyl)acetamide tartrate; (384) N-(4-Fluorobenzyl)-2-(4-isobutoxyphenyl)-N-{1-[2-(2-oxo-imidazolidin-1-yl)ethyl]piperidin-4-yl}acetamide hydrochloride; (385) 2-(4-Methoxyphenyl)-N-(4-methylbenzyl)-N-{1-[2-(2-oxo-imidazolidin-1-yl)ethyl]piperidin-4-yl}acetamide hydrochloride; (386) N-(4-Fluorobenzyl)-2-(4-isopropoxyphenyl)-N-{1-[2-(2-oxo-imidazolidin-1-yl)ethyl]piperidin-4-yl}acetamide hydrochloride; (387) N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide hydrochloride; (388) N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide hydrochloride; (389) 2-(4-Methoxyphenyl)-N-(4-methylbenzyl)-N-{1-[3-(2-oxo-2,3-dihydrobenzoimidazol-1-yl)propyl]piperidin-4-yl}-acetamide hydrochloride; (390) N-(4-Fluorobenzyl)-2-(4-isopropoxyphenyl)-N-{1-[4-(2-oxo-2,3-dihydrobenzoimidazol-1-yl)butyl]piperidin-4-yl}acetamide hydrochloride; (391) N-{1-[2-(2,4-Dioxo-1,4-dihydro-2H-quinazolin-3-yl)ethyl]piperidin-4-yl}-N-(4-fluorobenzyl)-2-(4-isopropoxyphenyl)acetamide hydrochloride; (392) 4-(4-Fluorobenzylamino)-piperidine-1-carboxylic acid benzyl ester; (393) N-(1-Benzyloxycarbonylpiperidin-4-yl)-N-(4-fluorobenzyl)-N′-(4-isopropoxybenzyl)carbamide; (394) N-(4-Fluorobenzyl)-N′-(4-isopropoxybenzyl)-N-piperidin-4-yl-carbamide oxalate; (395) N-{1-[2-(1,3-Dioxolan-2-yl)ethyl]piperidin-4-yl}-N-(4-fluorobenzyl)-N′-(4-isopropoxy-benzyl)carbamide oxalate; (396) N-{1-[2-(1,3-Dioxolan-2-yl)ethyl]piperidin-4-yl}2-(4-methoxyphenyl)-N-(4-methylbenzyl)acetamide hydrochloride; (397) N-{1-[2-(1,3-Dioxolan-2-yl-)ethyl]piperidin-4-yl}-N-(4-fluorobenzyl)-2-(4-isobutoxyphenyl)acetamide hydrochloride; (398) N-{1-[2-(1,3-Dioxolan-2-yl)ethyl]piperidin-4-yl}-2-(4-isopropoxyphenyl)-N-(4-methylbenzyl)acetamide hydrochloride; (399) N-{1-[2-(1,3-Dioxolan-2-yl)ethyl]piperidin-4-yl}-N-(4-fluorobenzyl)-2-(4-propoxyphenyl)acetamide tartrate; (400) N-(4-Fluorobenzyl)-N′-(4-isopropoxybenzyl)-N-{1-[2-((S)-4-methyl-1,3-dioxolane-2-yl)ethyl]piperidin-4-yl}carbamide oxalate; (401) N-(4-Fluorobenzyl)-N′-(4-isopropoxybenzyl)-N-[1-(3-morpholin-4-yl-propyl)piperidin-4-yl]carbamide oxalate; (402) 2-(4-Methoxyphenyl)-N-(4-methylbenzyl)-N-[1-(2-morpholin-4-yl-ethyl)piperidin-4-yl]acetamide dihydrochloride; (403) 2-(4-Methoxyphenyl)-N-(4-methylbenzyl)-N-[1-(3-morpholin-4-ylpropyl)piperidin-4-yl]acetamide dihydrochloride; (404) N-(4-Fluorobenzyl)-2-(4-isobutoxyphenyl)-N-[1-(3-morpholin-4-ylpropyl)piperidin-4-yl]acetamide dihydrochloride; (405) N-(4-Fluorobenzyl)-2-(4-isopropoxy-phenyl)-N-[1-(3-morpholin-4-yl-propyl)piperidin-4-yl]acetamide dihydrochloride; (406) N-(4-Fluorobenzyl)-N′-(4-isopropoxybenzyl)-N-[1-(3-piperidin-1-yl-propyl)piperidin-4-yl]carbamide oxalate; (407) N-(4-Fluorobenzyl)-N′-(4-isopropoxybenzyl)-N-[1-(3-((S)-4-isopropyl-2-oxazolidinon-1-yl-propyl)piperidin-4-yl]carbamide tartrate; (408) N-(4-Fluorobenzyl)-N′-(4-isopropoxybenzyl)-N-{1-[2-(2,5,5-trimethyl-1,3-dioxan-2-yl)ethyl]}piperidin-4-yl]carbamide oxalate; (409) N-{1-[3-(1,3-Dioxolan-2-yl)propyl]piperidin-4-yl}-N-(4-fluorobenzyl)-N′-(4-isopropoxybenzyl)carbamide oxalate; (410) N-[2,2-Dimethyl-1,3-dioxan-5-yl)-piperidin-4-yl]-N-(4-fluorobenzyl)-N′-(4-isopropoxybenzyl)carbamide oxalate; (411) N-(4-Fluorobenzyl)-N′-(4-isopropoxybenzyl)-N-{[2-(1-methylpyrrolidin-2-yl)ethyl]-piperidin-4-yl}carbamide oxalate; (412) N-[1-(2,2-Dimethyl-1,3-dioxan-5-yl)piperidin-4-yl]-N-(4-fluorobenzyl)-2-(4-isobutoxyphenyl)acetamide oxalate; (413) N-[1-(1,3-Dioxan-5-yl)-piperidin-4-yl)-N-(4-fluorobenzyl)-2-(4-isobutoxyphenyl)acetamide tartrate; (414) N-[1-(2,2-Dimethyl-1,3-dioxan-5-yl)piperidin-4-yl]-N-(4-fluorobenzyl)-2-(4-fluorophenyl)acetamide tartrate; (415) N-{1-[2-(1,3-Dioxan-4-yl)ethyl]piperidin-4-yl}-N-(4-fluorobenzyl)-2-(4-fluorophenyl)acetamide tartrate: (416) N-{1-[2-(1,3-Dioxan-4-yl)ethyl]piperidin-4-yl}-N-(4-fluorobenzyl)-2-(4-trifluoromethoxyphenyl)acetamide tartrate: (417) N-{1-[2-(1,3-Dioxan-4-yl)ethyl]piperidin-4-yl}-N-(4-fluorobenzyl)-2-(4-propoxyphenyl)acetamide tartrate; (418) N-(4-Fluorobenzyl)-2-(4-isobutoxyphenyl)-N-[1-(tetrahydropyran-4-yl)piperidin-4-yl]acetamide tartrate; (419) N-(4-Fluorobenzyl)-2-(4-isobutoxyphenyl)-N-[1-(tetrahydropyran-4-ylmethyl)piperidin-4-yl]acetamide tartrate; (420) N-(4-Fluorobenzyl)-2-(4-isobutoxyphenyl)-N-{1-[2-(tetrahydropyran-4-yl)ethyl]piperidin-4-yl]acetamide tartrate; (421) N-(4-Fluorobenzyl)-2-(4-fluorophenyl)-N-[1-(tetrahydropyran-4-yl)piperidin-4-yl]acetamide tartrate; (422) N-[1-((S)-3,5-Dihydroxypentyl)piperidine-4-yl]-N-(4-fluorobenzyl)-2-(4-isobutoxyphenyl)acetamide tartrate; (423) N-{1-[2-((4S)-1,3-Dioxane-4-yl)ethyl]piperidine-4-yl}-N-(4-fluorobenzyl)-2(4-isobutoxyphenyl)acetamide tartrate; (424) N-{1-[2-(1,3-Dioxan-2-yl)ethyl]piperidin-4-yl}-N-(4-fluorobenzyl)amine; (425) 2-(4-Benzyloxyphenyl)-N-{1-[2-(1,3-dioxan-2-yl)ethyl]piperidin-4-yl}-N-(4-fluorobenzyl)acetamide tartrate; (426) N-{1-[2-(1,3-Dioxan-2-yl)ethyl]piperidin-4-yl}-N-(4-fluorobenzyl)-2-(4-hydroxyphenyl)-acetamide tartrate; (427) N-{1-[2-(1,3-Dioxan-2-yl)ethyl]piperidin-4-yl}-N-(4-fluorobenzyl)-2-(4-methoxyphenyl)-acetamide tartrate; (428) N-{1-[2-(1,3-Dioxan-2-yl)ethyl]piperidin-4-yl}-N-(4-fluorobenzyl)-2-(4-isopropylphenyl)-acetamide tartrate; (429) N-{1-[2-(1,3-Dioxan-2-yl)ethyl]piperidin-4-yl}-N-(4-fluorobenzyl)-2-(4-trifluoromethoxy-phenyl)acetamide tartrate; (430) N-{1-[2-(1,3-Dioxan-2-yl)ethyl]-piperidin-4-yl}-N-(4-fluorobenzyl)-2-(4-ethoxyphenyl)-acetamide oxalate; (431) N-{1-[2-(1,3-Dioxan-2-yl)ethyl]piperidin-4-yl}-N-(4-fluorobenzyl)-2-(4-isopropoxyphenyl)-acetamide oxalate; (432) N-{1-[2-(1,3-Dioxan-2-yl)ethyl]piperidin-4-yl}-N-(4-fluorobenzyl)-2-phenylacetamide oxalate; (433) N-{1-[2-(1,3-Dioxan-2-yl)ethyl]piperidin-4-yl}-N-(4-fluorobenzyl)-2-[4-(2-fluoroethoxy)-phenyl]acetamide oxalate; (434) N-{1-[2-(5,5-Dimethyl-1,3-dioxan-2-yl)ethyl]piperidin-4-yl}-N-(4-fluorobenzyl)-2-(4-isobutoxyphenyl)acetamide oxalate; (435) N-(4-Fluorobenzyl)-2-(4-isobutoxyphenyl)-N-{1-[2-((R)-4-methyl-1,3-dioxan-2-yl)ethyl]-piperidin-4-yl}acetamide oxalate; (436) N-(4-Fluorobenzyl)-2-(4-isobutoxyphenyl)-N-{1-[2-((S)-4-methyl-1,3-dioxolan-2-yl)ethyl]piperidin-4-yl}acetamide oxalate; (437) N-{1-[2-(4,6-Dimethyl-1,3-dioxan-2-yl)ethyl]piperidin-4-yl}-N-(4-fluorobenzyl)-2-(4-isobutoxyphenyl)acetamide oxalate; (438) N-(4-Fluorobenzyl)-N-{1-[2-((S)-4-methyl-1,3-dioxolan-2-yl)ethyl]piperidin-4-yl}-2-(4-trifluoromethoxyphenyl)acetamide oxalate; (439) N-(4-Fluorobenzyl)-2-(4-isopropylphenyl)-N-{1-[2-((S)-4-methyl-1,3-dioxolan-2-yl)ethyl]-piperidin-4-yl}acetamide oxalate; (440) N-(4-Fluorobenzyl)-N-{1-[2-((R)-4-methyl-1,3-dioxan-2-yl)ethyl]piperidin-4-yl}-2-(4-trifluoromethoxyphenyl)acetamide oxalate; (441) N-(4-Fluorobenzyl)-2-(4-isobutoxyphenyl)-N-{1-[2-(2,5,5-trimethyl-1,3-dioxan-2-yl)ethyl]piperidin-4-yl}acetamide oxalate; (442) N-(4-Fluorobenzyl)-2-(4-isobutoxyphenyl)-N-{1-[2-(2-methyl-1,3-dioxolan-2-yl)ethyl]-piperidin-4-yl-}acetamide oxalate; (443) N-(4-Fluorobenzyl)-2-(4-isobutoxyphenyl)-N-{1-[3-(1,3-dioxolan-2-yl)propyl]piperidin-4-yl}acetamide tartrate; (444) N-(4-Fluorobenzyl)-2-(4-isobutoxyphenyl)-N-{1-(3-piperidin-1-yl-propyl)piperidin-4-yl}-acetamide dihydrochloride; (445) N-(4-Fluorobenzyl)-2-(4-isobutoxyphenyl)-N-{1-[2-(tetrahydropyran-2-yloxy)ethyl]-piperidin-4-yl}acetamide oxalate; (446) N-(4-Fluorobenzyl)-2-(4-isobutoxyphenyl)-N-{1-[3-(2-oxo-piperidin-1-yl)propyl]piperidin-4-yl}acetamide; (447) N-(4-Fluorobenzyl)-2-(4-isobutoxyphenyl)-N-{1-[3-(2-oxo-pyrrolidin-1-yl)propyl]piperidin-4-yl}acetamide hydrochloride; (448) N-(4-Fluorobenzyl)-2-(4-isobutoxyphenyl)-N-{1-[3-((R)-4-isopropyl-2-oxo-oxazolidin-3-yl)propyl]piperidin-4-yl}acetamide oxalate; (449) N-(4-Fluorobenzyl)-2-(4-isobutoxyphenyl)-N-{1-[3-(2-oxo-oxazolidin-3-yl)propyl]piperidin-4-yl}acetamide oxalate; (450) N-(4-Fluorobenzyl)-2-(4-isobutoxyphenyl)-N-{1-[3-((S)-4-methyl 2-oxo-oxazolidin-3-yl)propyl]piperidin-4-yl}acetamide tartrate; (451) N-(4-Fluorobenzyl)-2-(4-isobutoxyphenyl)-N-{1-[3-((S)-4-ethyl-2-oxo-oxazolidin-3-yl)-propyl]piperidin-4-yl}acetamide oxalate; (452) N-(4-Fluorobenzyl)-2-(4-isobutoxyphenyl)-N-{1-[2-(1,3-oxothiolan-2-yl)ethyl]piperidin-4-yl}acetamide L-tartrate; (453) 2-(4-Bromophenyl)-N-{1-[2-(1,3-dioxan-2-yl)ethyl)piperidin-4-yl}-N-(4-fluorobenzyl)-acetamide L-tartrate; (454) N-{1-[2-(1,3-Dioxan-2-yl)ethyl)piperidin-4-yl}-N-(4-fluorobenzyl)-2-(4-isobutylamino-phenyl)acetamide L-tartrate; (455) N-{1-[2-(1,3-Dioxan-2-yl)ethyl)piperidin-4-yl}-N-(4-fluorobenzyl)-2-(4-propylaminophenyl)acetamide L-tartrate; (456) N-{1-[2-(1,3-Dioxan-2-yl)ethyl)piperidin-4-yl}-N-(4-fluorobenzyl)-2-(4-(1-nitropropyl)-phenyl)acetamide L-tartrate; (457) N-{1-[2 (1,3-Dioxan-2-yl)ethyl)piperidin-4-yl}-N-(4-fluorobenzyl)-2-[4-(2-oxopyrrolidin-1-yl)phenyl)acetamide L-tartrate; (458) N-{1-[2-(1,3-Dioxan-2-yl)ethyl)piperidin-4-yl}-N-(4-fluorobenzyl)-2-(4-isobutylsulfanylphenyl)acetamide L-tartrate; (459) N-{1-[2-(1,3-Dioxan-2-yl)ethyl)piperidin-4-yl}-N-(4-fluorobenzyl)-2-(4-iodophenyl)-acetamide L-tartrate; (460) 2-(4-Acetophenyl)-N-{1-[2-(1,3-dioxan-2-yl)ethyl)piperidin-4-yl}-N-(4-fluorobenzyl)-acetamide L-tartrate; (461) 2-[4-(1-hydroxyiminoethyl)phenyl]-N-{1-[2-(1,3-dioxan-2-yl)ethyl)piperidin-4-yl}-N-(4-fluorobenzyl)acetamide L-tartrate; (462) N-{1-[2-(1,3-Dioxan-2-yl)ethyl)piperidin-4-yl}-N-(4-fluorobenzyl)-2-(4-morpholin-4-yl-phenyl)acetamide L-tartrate; (463) N-{1-[2-(1,3-Dioxan-2-yl)ethyl)-piperidin-4-yl}-N-(4-fluorobenzyl)-2-(4-pyrazol-1-ylphenyl)acetamide L-tartrate; (464) N-{1-[2-(1,3-Dioxan-2-yl)-1-methylethyl]piperidin-4-yl}-N-(4-fluorobenzyl)-2-(4-isobutoxyphenyl)-acetamide L-tartrate; (465) N-{1-[2-(1,3-Dioxan-4-yl)ethyl)piperidin-4-yl}-N-(4-fluorobenzyl)-2-(4-pyrazol-1-ylphenyl)acetamide L-tartrate; (466) N-[1-((R)-3,5-Dihydroxypentyl)pipe-ridine-4-yl]-N-(4-fluorobenzyl)-2-(4-isobutoxyphenyl)acetamide tartrate; (467) N-{1-[2-((4R)-1,3-Dioxane-4-yl)ethyl]piperidine-4-yl}-N-(4-fluorobenzyl)-2-(4-isobutoxyphenyl)acetamide tartrate; (468) N-{1-[2-(1,3-Dioxan-2-yl)ethyl]piperidin-4-yl}-N-(4-fluorobenzyl)-2-[4-(1,2,4-triazol-4-yl)phenyl]acetamide L-tartrate; (469) nortriptyline; (470) duloxetine; (471) lofepramine; (472) tomoxetine; (473) 3-({1-[2-(7-methyl-5-oxo-5H)-[1,3]thiazolo[3,2-a]pyrimidin-6-yl)ethyl]-3-pyrrolidinyl}methyl)-1H-indole-5-carbonitrile hydrochloride; (474) 3-({1-[2-(6-chloro-2-oxo-2,3-dihydro-1H-indol-5-yl)ethyl]-3-pyrrolidinyl}-methyl)-1H-indole-5-carbonitrile hydrochloride; (475) moclobemide; (476) N-acetylserotonin; (477) bromfaromine; (478) beflaxozone; (479) chlorimipramine; (480) cyanimipramine; (481) cianopramine; (482) desipramine; (483) protriptyline; (484) trimipramine; (485) doxepin; (486) cyclobenzaprine; (487) 5-methoxycarbonylamino-N-acetyltryptamine; (488) amoxapine; (489) maprotiline; (490) fefazodone; (491) flesinoxan hydrochloride; (492) urapidil; (493) WY47846 (3a,4,4a,6a,7,7a-hexahydro-2-[4-[4-(2-pyrimidinyl)-1-piperazinyl]-butyl]-4,7-etheno-1H-cyclobutano[f]isoindole-1,3(2H)-dione dihydrochloride sesquihydrate); (494) SM3997 (N-[4-[4-(2-pyrimidinyl)-1-piperazinyl]butyl]-bicyclo[2.2.1]heptane-2,3-di-exo-carboximide); (495) 2-(4-(4-(2-pyrimidinyl)-1-piperazinyl-propyl)-1,2-benzoisothiazol-3-(2H)-one 1,1-dioxide hydrochloride; (496) KC9172 (3-butyl-9,9-dimethyl-7-[4-[4-[2-methoxyphenyl)-1-piperazinyl]butyl]-3,7-diazabicyclo[3,2,1]nonane-2,4,6,8-tetraone); (497) 4-(N,N-dipropylamino)-6-methoxy-1,3,4,5-tetrahydrobenz-[c,d]indole; (498) 4-[4-(N-1,2-benzisothiazol-3(2H)-one 1,1-dioxido)]butylamino-6-methoxy-1,3,4,5-tetrahydrobenz[c,d]-indole hydrochloride; (499) 5-carboxamidotryptamine; (500) N,N-dipropyl-5-carboxamidotryptamine; (501) AH25086 (3-(2-aminoethyl)-1H-indole-5-(N-methyl)acetamide); (502) GR43175 (3-(2-dimethylaminoethyl)-1H-indole-5-(N-methyl)methanesulfonamide); (503) 3-(2-[4-[2-(1,2-benzisothiazole-3(2H)-one 1,1-dioxido)]butyl]amino)ethyl-5-methoxy-1H-indole; (504) spiroxatrine; (505) MDL72832 (8-[4-(1,4-benzodioxan-2-ylmethylamino)butyl]-8-azaspiro-[4,5]decane-7,9-dione); (506) 2-[4-(1,4-benzodioxan-2-ylmethylamino)butyl]-1,2-benzisothiazol-3(2H)-one 1,1-dioxide; (507) 2-(N,N-dipropylamino)-8-hydroxy-1,2,3,4-tetrahydronaphthalene; (508) 2-{4-(2-(1,2-benzisothiazol-3(2H)-one 1,1-dioxido)ibutyl}amino-8-methoxy-1,2,3,4-tetrahydronaphthalene; (509) 3-N,N-dipropylamino-5-hydroxy-thiochroman; 3-N,N-dipropylamino-5-ethoxy-thiochroman; (510) 3-N,N-dipropylamino-5-ethoxychroman; (511) 1-[2-(3-indolyl)]-ethyl-2,6-dimethyl-piperidine; (512) 1-{2-[3-(5-carboxamido)indolyl]}ethyl-2,6-dimethylpiperidine; (513) RU24924 (5-methoxy-3-(1,2,3,6-tetrahydropyridin-4-yl]-1H-indole); (514) 5-methoxy-3-(1,2,3,6-tetrahydropyridin-5-yl)-1H-indole; (515) diethyl N-benzyloxycarbonyl-5-benzyloxycarbonyloxy-L-tryptophyl-L-aspartate; (516) dibenzyl N-benzyloxycarbonyl-5-hydroxy-L-tryptophanylaspartate; (517) 5-Hydroxy-L-tryptophyl-L-aspartic acid trihydrate; (518) diethyl N-benzyloxycarbonyl-5-hydroxy-L-tryptophyl-L-glutamate; (519) diethyl 5-hydroxy-L-tryptophyl-L-glutamate hydrochloride; (520) dibenzyl L-benzyloxycarbonyl-5-hydroxytryptophyl-L-glutamate; (521) 5-hydroxy-L-tryptophyl-L-glutamic acid; (522) pentachlorophenyl ester of N-benzyloxycarbonyl-5-hydroxy-L-tryptophan; (523) methyl ester of N-benzyloxycarbonyl-5-hydroxy-L-tryptophyl-L-tyrosine; (524) N-Acetyl-5-hydroxy-L-tryptophan; (525) methyl ester of N-acetyl-5-hydroxy-L-tryptophyl-L-tyrosine; (526) methyl ester of N-acetyl-5-hydroxy-L-tryptophyl-5-hydroxy-L-tryptophan; (527) 5-hydroxy-L-tryptophyl-L-alanine hydrate; (528) 5-hydroxy-L-tryptophan-L-valine; (529) 5-hydroxy-L-tryptophyl-L-leucine; (530) 5-hydroxy-L-tryptophyl-L-proline; (531) 5-hydroxy-L-tryptophyl-L-phenylalanine; (532) 5-hydroxy-L-tryptophyl-5-hydroxy-L-tryptophan; (533) 5-hydroxy-L-tryptophyl-L-tryptophan; (534) 1-(5-hydroxy)tryptophyl-L-serine; (535) 5-hydroxy-L-tryptophyl-L-arginine; (536) 5-hydroxy-L-tryptophylglycine; (537) 5-hydroxy-1-tryptophyl-gamma-aminobutyric acid; (538) 5-hydroxy-L-tryptophanamide hydrate; (539) methyl ester of 5-hydroxy-L-tryptophyl-L-histidine; (540) benzyl ester of L-5-hydroxytryptophan; (541) benzyl ester of N-benzyloxycarbonyl-5-hydroxy-L-tryptophyl-5-hydroxy-L-tryptophan; (542) 5-Hydroxy-L-tryptophyl-5-hydroxy-L-tryptophan hemihydrate; (543) 5-hydroxytryptophan inosinate; (544) theophylline salt of (DL) 5-hydroxytryptophan; (545) RU25591 (6,7,8,9-tetrahydro N,N-dimethyl 5-[4-nitrophenyl]oxy 5H-benzocyclohepten 7-amine) cis-fumarate); (546) LM5008 (4-[2-(3-indolyl)ethyl]piperidine); (547) DU24565 (6-nitro-2-(1-piperazinyl)quinoline); (548) CGP6085/A (4-(5,6-dimethyl-2-benzofuranyl)piperidine hydrochloride); (549) alaprociate; (550) dibenzoxazepine; (551) deprenyl; (552) isocarboxazide; (553) furazolidone; (554) procarbazine; (555) Ro 60-0175/ORG 35030 ((S)-2-(4,4,7-trimethyl-1,4-dihydro-indeno (1,2-B) pyrrol-1-yl)-1-methyl-ethylamine) (556) Ro 60-0332/ORG 35035 ((S)-2-(Chloro-5-fluoro-indol-1-yl)-1-methylethylamine); (557) 1-[6-Chloro-5-trifluoromethyl)-2-pyridinyl]-piperazine hydrochloride; (558) 5-carboxyamidotryptamine; (559) SB 206553 (3,5-Dihydro-5-methyl-N-3-pyridinylbenzo[1,2-b:4,5-b′]dipyrrole-1(2H)-carboxamide hydrochloride); (560) ondansetron; (561) granisetron; (562) tropisetron; (563) dolasetron; (564) palonosetron; (565) trimethobenzamide; (566) risperidone; (567) clozapine; (568) azatadine; (569) cyproheptadine; (570) fenclonine; (571) chlorpromazine; (572) (3β)-2,3-dihydrolysergine; (573) (3β)-2,3-dihydroisolysergine; (574) (3β, 5β, 8β)-9,10-didehydro-2,3-dihydro-6-methylergoline-8-acetonitrile; (575) 25I-NBMD (2-(4-iodo-2,5-dimethoxyphenyl)-N-[(2,3-methylenedioxyphenyl)methyl]ethanamine); (576) N-(2-methoxybenzyl)-1-(8-bromo-2,3,6,7-tetrahydrobenzo[1,2-b:4,5-b′]difuran-4-yl)-2-aminoethane; (577) 5-benzyloxytryptamine; (578) 5-methoxy-7-N,N-dimethyltryptamine; (579) A372159 ((11S,16R)-3-[4-(propan-2-yloxy)-2-(trifluoromethyl)phenyl]-6-oxa-10,14-diazatetracyclo[8.6.1.0^{5,17}.0^{11,16}]heptadeca-1,3,5(17)-triene); (580) AL-34662 (1-((S)-2-Aminopropyl)-1H-indazol-6-ol); (581) AL-37350A ((S)-(+)-1-(2-Aminopropyl)-8,9-dihydropyrano[3,2-e]indole); (582) AL-38022A ((S)-2-(8,9-dihydro-7H-pyrano[2,3-g]indazol-1-yl)-1-methylethylamine); (583) AS-19 ((2S)—N,N-dimethyl-5-(1,3,5-trimethylpyrazol-4-yl)-1,2,3,4-tetrahydronaphthalen-2-amine); (584) alnespirone; (585) BIMU8 (N-[(1R,5S)-8-methyl-8-azabicyclo[3.2.1]oct-3-yl]-2-oxo-3-(propan-2-yl)-2,3-dihydro-1H-benzimidazole-1-carboxamide hydrochloride); (586) BMY-14802 (1-(4-fluorophenyl)-4-[4-(5-fluoropyrimidin-2-yl)piperazin-1-yl]butan-1-01); (587) BRL-54443 (3-(1-methylpiperidin-4-yl)-1H-indol-5-01); (588) batoprazine; (589) benzylpiperazine; (590) binospirone; (591) 1-(8-bromobenzo[1,2-b;4,5-b]difuran-4-yl)-2-aminopropane); (592) CP-809,101 (2-[(3-Chlorophenyl)methoxy]-6-(1-piperazinyl)pyrazine); (593) CP-93,129 (3-(1,2,3,6-tetrahydropyridin-4-yl)-1,4-dihydropyrrolo[3,2-b]pyridin-5-one); (594) CP-94,253 (3-(1,2,5,6-tetrahydro-4-pyridyl)-5-propoxypyrrolo[3,2-b]pyridine); (595) CGS-12066A (4-(4-methylpiperazin-1-yl)-7-(trifluoromethyl)pyrrolo[1,2-a]quinoxaline); (596) chlorophenylbiguanide; (597) chlorphentermine; (598) dazopride; (599) dimemebfe; (600) 2,5-dimethoxy-4-bromoamphetamine; (601) 2,5-dimethoxy-4-fluoroamphetamine; (602) 2,5-dimethoxy-4-methylamphetamine; (603) EMD-386,088 (5-chloro-2-methyl-3-(1,2,3,6-tetrahydro-4-pyridinyl)-1H-indole); (604) EMDT (2-(2-ethyl-5-methoxy-1-indol-3-yl)-N,N-dimethylethanamine); (605) p-fluoropiperazine; (606) fluprazine; (607) jimscaline; (608) LY-293,284 ((4R)-6-acetyl-4-(di-n-propylamino)-1,3,4,5-tetrahydrobenz[c,d]indole); (609) lasmitidan; (610) lorcaserin; (611) 2-methyl-5-hydroxytryptamine; (612) 2-methyl-4,5-methylenedioxyamphetamine; (613) NBUMP(N-[4-[4-(2-methoxyphenyl)piperazin-1-yl]butyl]adamantane-1-carboxamide); (614) 1-(1-naphthyl)piperazine; (615) Org-37,684 ((3S)-3-[(2,3-dihydro-5-methoxy-1H-inden-4-yl)oxy]pyrrolidine); (616) PNU-22394 (6-Methyl-1,2,3,4,5,6-hexahydro-azepino[4,5-b]indole)); (617) PRX-00023 (N-(3-[4-(4-cyclohexylmethanesulfonylaminobutyl)piperazin-1-yl]phenyl)acetamide); (618) RH-34 (3-[2-(2-methoxybenzylamino)ethyl]-1H-quinazoline-2,4-dione); (619) RS56812 (N-[(3R)-1-azabicyclo[2.2.2]oct-3-yl]-2-(1-methyl-1H-indol-3-yl)-2-oxoacetamide); (620) RS67333 (1-(4-amino-5-chloro-2-methoxyphenyl)-3-(1-butyl-4-piperidinyl)-1-propanone); (621) RU24969 (5-Methoxy-3-(1,2,5,6-tetrahydro-4-pyridinyl)-1H-indole); (622) Ro60-0175 ((S)-6-Chloro-5-fluoro-1H-indole-2-propanamine); (623) TFMFly ((2R)-1-(8-trifluoromethyl-2,3,6,7-tetrahydrobenzo[1,2-b:4,5-b]difuran-4-yl)-2-aminoethane); (624) U92016-A ((8R)-8-(Dipropylamino)-6,7,8,9-tetrahydro-3H-benz[e]indole-2-carbonitrile) (625) VER3323 ((2S)-1-(6-bromo-2,3-dihydroindol-1-yl)propan-2-amine); (626) vilazodone; (627) WAY-181,187 (1-[(2S,5S)-4,4-difluoro-5-(hydroxymethyl)tetrahydrofuran-2-yl]pyrimidine-2,4(1H,3H)-dione); (628) WAY-208,466 (N′-[(2Z)-4-(2,4-dichlorophenyl)-3-(2-methylpropyl)-1,3-thiazol-2(3H)-ylidene]-2-(pyrazin-2-yloxy)acetohydrazide); (629) YM-348 (2S)-1-(7-ethyl-1H-furo[2,3-g]indazol-1-yl)propan-2-amine); (630) alprenolol; (631) BMY 7378 (8-(2-[4-(2-methoxyphenyl)-1-piperazinyl]ethyl)-8-azaspiro[4.5]decane-7,9-dione); (632) cyanopindolol; (633) iodocyanopindolol; (634) lezcotozan; (635) methiothepin; (636) NAN-190 (1-(2-methoxyphenyl)-4-(4-phthalimidobutyl)piperazine); (637) oxprenolol; (638) pindolol; (639) propranolol; (640) robalzotan; (641) S15535 (1-(2,3-dihydro-1,4-benzodioxin-8-yl)-4-(2,3-dihydro-1H-inden-2-yl)piperazine); (642) spiperone; (643) TFMPP; (644) UH-301 ((S)-5-fluoro-8-hydroxy-2-(dipropylamino)tetralin); (645) WAY-100,135 ((S)—N-tert-butyl-3-(4-(2-methoxyphenyl)-piperazin-1-yl)-2-phenylpropanamide); (646) WAY-100,635 (N-[2-[4-(2-methoxyphenyl)-1-piperazinyl]ethyl]-N-(2-pyridyl)cyclohexanecarboxamide); (647) mefway; (648) 5-hydroxytryptophan; (649) 5-hydroxytryptophan creatinine sulfate complex; (650) 5-methoxytryptamine; (651) 5-methoxytryptamine creatinine sulfate complex; (652) 5-HIAA (5-hydroxyindoleacetic acid); and (653) 5-HIAA (5-hydroxyindoleacetic acid) creatinine sulfate complex; and the salts, solvates, analogues, congeners, bioisosteres, hydrolysis products, metabolites, precursors, and prodrugs thereof.

All of the compounds mentioned above are known drugs and are readily available to the public. Some of them can be purchased from chemical companies, such as Sigma-Aldrich, St. Louis, Mo. Regimens for administering these drug compounds are well known and, if necessary, can be easily re-established. Effective doses will vary, as recognized by those skilled in the art, depending on the type or degree of the disease to be treated; the subject's size, weight, age, and sex; the route of administration; the excipient usage; and the possible co-usage with other therapeutic treatment. The daily dose of the compositions described above can be 5-5,000 mg (e.g., 10-2,500 or 10-3,000 mg) of the first agent, 1-5,000 mg (e.g., 2-1,000 or 2-3,000 mg) of the second agent, and 0.1-1,000 mg (e.g., 1-50 mg) of the third agent. Specifically, for a composition in which the first agent is metformin, the second agent is aspirin, and the third agent is melatonin, typically the daily dose of the first agent, second agent, and third agent is 5-5000 mg of metformin, 1-5000 mg aspirin, and 5-5000 mg of melatonin. Preferably, the daily dose of the first agent, second agent, and third agent is 5-1500 mg of metformin, 1-1000 mg aspirin, and 5-1500 mg of melatonin. More preferably, the daily dose of the first agent, the second agent, and the third agent is 5-1000 mg of metformin, 1-500 mg of aspirin, and 5-1000 mg of melatonin. Other dosage ranges can readily be determined by a treating physician or other medical professional.

In certain other embodiments of the invention, one or more active compounds of the present invention are associated with a carrier substance such as a compound or molecule (e.g., an antibody, antibody fragment, receptor, or other specific carrier), to facilitate the transport of the one or more active compounds to the intended site of action, such as a cell type or tissue involved in the disease or condition which a composition according to the present invention is intended to treat. In these embodiments, each of the first, second, and third agents can be associated with its own carrier substance, or more than one of the first, second, and third agents can be associated with a single carrier substance. For example, all of the first, second, and third agents can be associated with a single carrier substance. In another alternative, the first and second active agents can be associated with a single carrier substance, the first and third active agents can be associated with a single carrier substance, or the second and third active agents can be associated with a single carrier substance; in this alternative, the active agent that is not associated with the carrier substance with which the two other active agents are associated (i.e., the first and second active agents, the first and third active agents, or the second and third active agents, respectively) can be associated with its own separate carrier substance. The association between the first, second, and third agents and the carrier substance or carrier substances can be covalent or noncovalent, as is generally known in the art; peptide linkers or biotin-avidin or biotin-streptavidin linkages can be employed.

The first agent, second agent, or third agent can be either covalently or noncovalently bound to an individual carrier substance. Typically, however, the first agent, second agent, or third agent is covalently bound to an individual carrier substance. Methods for binding the first agent, second agent, or third agent to an individual carrier substance are known in the art. Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the 6-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the C-amino group of the side chain of lysine. Many other electrophilic reagents are known that will react with the 6-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail. Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996), incorporated herein by this reference.

The individual carrier substances can be, but are not limited to, antibodies, hormones, receptor agonists or antagonists, or receptors. As used herein, unless further defined or limited, the term “antibody” encompasses both polyclonal and monoclonal antibodies, as well as genetically engineered antibodies such as chimeric or humanized antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. Receptors are well known in the art and include G-protein coupled receptors (GPCRs). G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors. Agonists and antagonists specifically binding these receptors can be used as individual carrier substances; suitable receptors, agonists, or antagonists can be selected based on their specificity and the location of the receptors in particular cells or tissues.

In addition to the three required agents, the composition used in the methods described in this disclosure can include one or more additional active ingredients.

The composition can comprise a pharmaceutically acceptable carrier, as detailed below. This pharmaceutically acceptable carrier is not to be confused with the individual carrier substances individually bound to one or more of the first agent, the second agent, or the third agent as described above.

As detailed above, compositions according to the present invention can be used to treat metabolic syndrome and various other diseases. In addition to metabolic syndrome and diseases and conditions associated with metabolic syndrome, compositions according to the present invention can be used to treat hyperproliferative disease (including cancer), AIDS, Parkinson's disease, polycystic ovarian syndrome, Alzheimer's disease, osteoporosis, sleep apnea, erectile dysfunction, McArdle disease, and a carbohydrate metabolism disorder. Compositions according to the present invention can also be used to treat aging or fatigue.

The term “hyperproliferative disease” refers to a disease caused by excess cell proliferation that is not governed by the usual limitation of normal growth. A hyperproliferative disease can include benign tumors and malignant tumors. A hyperproliferative disease can include solid tumors. A “solid tumor”, as used herein, refers to an abnormal mass of tissue that usually does not contain cysts or liquid areas. Solid tumors can be benign (not cancerous) or malignant (cancerous).

One aspect of this invention features a method of administering an effective amount of one or more of the above-mentioned compositions to a subject for treating a disease described above. Such a subject can be identified by a health care professional based on results from any suitable diagnostic method. “An effective amount” refers to the amount of one or more compositions described above that is required to confer a therapeutic effect on a treated subject.

To practice the method of the present invention, one or more of the above-described compositions can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition for topical administration can be prepared in form of an ointment, a gel, a plaster, an emulsion, a lotion, a foam, a cream of a mixed phase or amphiphilic emulsion system (oil/water-water/oil mixed phase), a liposome, a transfersome, a paste, or a powder. Liposomes are known in the art; suitable proportions of ingredients for the preparation of liposomes are also known in the art and are described, for example, in European Patent Application Publication No. EP 1332755 by Weng et al., incorporated herein in its entirety by this reference. Typically, the composition is encapsulated by the liposome.

Any of the compositions described above can also be administered in the form of suppositories for rectal administration. It also can be designed such that the composition is released in the intestine. For example, the composition is confined in a solid sub-unit or a capsule compartment that have respectively a matrix or a wall or a closure comprising an enteric polymer which dissolves or disperses at the pH of the small or large intestine to release the drug substance in the intestine. Suitable such polymers have been described above, for example with reference to U.S. Pat. No. 5,705,189.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active thiophene compound. Examples of other carriers include colloidal silicon oxide, magnesium stearate, cellulose, sodium lauryl sulfate, and D&C Yellow #10.

The compositions described above can be used to treat diseases and conditions such as metabolic syndrome, Parkinson's disease, or polycystic ovarian syndrome. The diseases mentioned above also include their associated disorders. For example, disorders associated with metabolic syndrome include atherosclerosis, coronary heart disease, stroke, obesity, diabetes, atherogenic dyslipidemia (e.g., high triglyceride levels, low HDL cholesterol levels, and high LDL cholesterol levels), hypertension, insulin resistance, prothrombotic state (e.g., high fibrinogen or plasminogen activator inhibitor-1 levels), and proinflammatory state (e.g., elevated C-reactive protein levels).

The compositions described above can also be used to treat additional diseases and conditions, including hyperproliferative diseases and Alzheimer's disease. Hyperproliferative diseases include benign tumors and malignant tumors, as well as non-tumor hyperproliferative diseases. Benign tumors include, but are not limited to: adrenal tumors such as adenoma, adrenal pheochromocytoma and adrenal ganglioneuroma; brain tumors such as meningioma and adenoma; peripheral nerve tumors such as neurofibroma and schwannoma; liver tumors such as adenoma; thyroid tumors such as follicular adenoma; parathyroid tumors such as adenoma; thymus tumors such as thymoma; salivary gland tumors such as pleomorphic adenoma; small intestine tumors such as villous adenoma; colon tumors such as tubulovillous adenoma, adenomatous polyp of colon, and polyposis coli; pancreas tumors such as serous cystadenoma; islet tumors such as pancreatic islet cell tumor; nasopharyngeal tumors such as nasal angiofibroma; ovarian tumors such as atypical proliferating mucinous neoplasm, Brenner tumor of ovary, mucinous cystadenoma, papillary cystadenoma, dermoid cyst of ovary, ovarian teratoma, ovarian fibroma, luteoma, and struma ovarii; uteruine tumors such as uterine cellular leiomyoma and leiomyoma; placentaal tumors such as chorioangioma, partial hydatidiform mole, and complete hydatidiform mole; bone tumors such as cavernous hemangioma and giant cell tumor; soft tissue tumors such as cavernous hemangioma, desmoid tumor, lipoma, myelolipoma, and osteochondroma; joint tumors such as synovial chondromatosis; lung tumors such as carcinoid tumor, granular cell tumor, and hemangioma; myocardium tumors such as atrial myxoma; breast tumors such as fibroadenoma, intraductal papilloma and schwannoma; kidney tumors such as congenital mesoblastic nephroma; and skin tumors such as giant congenital intradermal nevus.

As used generally herein, the term “hyperproliferative disorders” refers to excess cell proliferation that is not governed by the usual limitation of normal growth. The term denotes malignant as well as nonmalignant cell populations. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Hyperproliferative cell disorders include tumors as well as non-tumor conditions. A “tumor” here refers to an abnormal mass of tissue that results from excessive cell division that is uncontrolled and progressive, also called a neoplasm.

Examples of tumors include a variety of solid tumors such as laryngeal tumors, brain tumors, other tumors of the head and neck; colon, rectal and prostate tumors; breast and thoracic solid tumors; ovarian and uterine tumors; tumors of the esophagus, stomach, pancreas, and liver; bladder and gall bladder tumors; skin tumors such as melanomas and the like; and a fluid tumor such as leukemia.

A “solid tumor,” as used herein, refers to an abnormal mass of tissue that usually does not contain cysts or liquid areas. Solid tumors may be benign (not cancerous) or malignant (cancerous). Solid tumors have a distinct structure that mimics that of normal tissues and comprises two distinct but interdependent compartments: the parenchyma (neoplastic cells) and the stroma that the neoplastic cells induce and in which they are dispersed. Different types of solid tumors are named for the type of cells that form them. Examples of solid tumors are sarcomas, carcinomas, and lymphomas. Solid tumors are loci of tumor cells in which the majority of cells are tumor cells or tumor-associated cells.

More particularly, “tumor” as used herein refers to either benign (non-cancerous) or malignant tumors.

Malignant tumors include, but are not necessarily limited to: (A) breast cancer, including: (1) ductal carcinoma, including ductal carcinoma in situ (DCIS) (comedocarcinoma, cribriform, papillary, micropapillary), infiltrating ductal carcinoma (IDC), tubular carcinoma, mucinous (colloid) carcinoma, papillary carcinoma, metaplastic carcinoma, and inflammatory carcinoma; (2) lobular carcinoma, including lobular carcinoma in situ (LCIS) and invasive lobular carcinoma; and (3) Paget's disease of the nipple; (B) cancers of the female reproductive system, including: (1) cancers of the cervix uteri, including cervical intraepithelial neoplasia (Grade I), cervical intraepithelial neoplasia (Grade II), cervical intraepithelial neoplasia (Grade III) (squamous cell carcinoma in situ), keratinizing squamous cell carcinoma, nonkeratinizing squamous cell carcinoma, verrucous carcinoma, adenocarcinoma in situ, adenocarcinoma in situ, endocervical type, endometrioid adenocarcinoma, clear cell adenocarcinoma, adenosquamous carcinoma, adenoid cystic carcinoma, small cell carcinoma, and undifferentiated carcinoma; (2) cancers of the corpus uteri, including endometrioid carcinoma, adenocarcinoma, adenocanthoma (adenocarcinoma with squamous metaplasia), adenosquamous carcinoma (mixed adenocarcinoma and squamous cell carcinoma, mucinous adenocarcinoma, serous adenocarcinoma, clear cell adenocarcinoma, squamous cell adenocarcinoma, and undifferentiated adenocarcinoma; (3) cancers of the ovary, including serous cystadenoma, serous cystadenocarcinoma, mucinous cystadenoma, mucinous cystadenocarcinoma, endometrioid tumor, endometrioid adenocarcinoma, clear cell tumor, clear cell cystadenocarcinoma, and unclassified tumor; (4) cancers of the vagina, including squamous cell carcinoma and adenocarcinoma; and (5) cancers of the vulva, including vulvar intraepithelial neoplasia (Grade I), vulvar intraepithelial neoplasia (Grade II), vulvar intraepithelial neoplasia (Grade III) (squamous cell carcinoma in situ); squamous cell carcinoma, verrucous carcinoma, Paget's disease of the vulva, adenocarcinoma (NOS), basal cell carcinoma (NOS), and Bartholin's gland carcinoma; (C) cancers of the male reproductive system, including: (1) cancers of the penis, including squamous cell carcinoma; (2) cancers of the prostate, including adenocarcinoma, sarcoma, and transitional cell carcinoma of the prostate; (3) cancers of the testis, including seminomatous tumor, nonseminomatous tumor, teratoma, embryonal carcinoma, yolk sac tumor, and choriocarcinoma; (D) cancers of the cardiac system, including sarcoma (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma, and squamous cell carcinoma of the pharynx; (F) cancers of the lung, including squamous cell carcinoma (epidermoid carcinoma), variants of squamous cell carcinoma, spindle cell carcinoma, small cell carcinoma, carcinoma of other cells, carcinoma of intermediate cell type, combined oat cell carcinoma, adenocarcinoma, acinar adenocarcinoma, papillary adenocarcinoma, bronchiolo-alveolar carcinoma, solid carcinoma with mucus formation, large cell carcinoma, giant cell carcinoma, clear cell carcinoma, and sarcoma; (G) cancers of the gastrointestinal tract, including: (1) cancers of the ampulla of Vater, including primary adenocarcinoma, carcinoid tumor, and lymphoma; (2) cancers of the anal canal, including adenocarcinoma, squamous cell carcinoma, and melanoma; (3) cancers of the extrahepatic bile ducts, including carcinoma in situ, adenocarcinoma, papillary adenocarcinoma, adenocarcinoma, intestinal type, mucinous adenocarcinoma, clear cell adenocarcinom, segnet-ring cell carcinoma, adenosquamous carcinoma, squamous cell carcinoma, small cell (oat) carcinoma, undifferentiated carcinoma, carcinoma (NOS), sarcoma, and carcinoid tumor; (4) cancers of the colon and rectum, including adenocarcinoma in situ, adenocarcinoma, mucinous adenocarcinoma (colloid type; greater than 50% mucinous carcinoma), signet ring cell carcinoma (greater than 50% signet ring cell), squamous cell (epidermoid) carcinoma, adenosquamous carcinoma, small cell (oat cell) carcinoma, undifferentiated carcinoma, carcinoma (NOS), sarcoma, lymphoma, and carcinoid tumor; (5) cancers of the esophagus, including squamous cell carcinoma, adenocarcinoma, leiomyosarcoma, and lymphoma; (6) cancers of the gallbladder, including adenocarcinoma, adenocarcinoma, intestinal type, adenosquamous carcinoma, carcinoma in situ, carcinoma (NOS), clear cell adenocarcinoma, mucinous adenocarcinoma, papillary adenocarcinoma, signet-ring cell carcinoma, small cell (oat cell) carcinoma, squamous cell carcinoma, and undifferentiated carcinoma; (7) cancers of the lip and oral cavity, including squamous cell carcinoma; (8) cancers of the liver, including hepatoma (hepatocellular carcinoma), cholangiocarcinoma, hepatoblastoma, angiosarcoma, hepatocellular adenoma, and hemangioma; (9) cancers of the exocrine pancreas, including duct cell carcinoma, pleomorphic giant cell carcinoma, giant cell carcinoma, osteoclastoid type, adenocarcinoma, adenosquamous carcinoma, mucinous (colloid) carcinoma, cystadenocarcinoma, acinar cell carcinoma, papillary carcinoma, small cell (oat cell) carcinoma, mixed cell typed, carcinoma (NOS), undifferentiated carcinoma, endocrine cell tumors arising in the islets of langerhans, and carcinoid; (10) cancers of the salivary glands, including acinic (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well differentiated or low grade), and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenosquamous carcinoma, squamous cell carcinoma, small cell carcinoma, undifferentiated carcinoma, lymphoma, sarcoma, and carcinoid tumor; and (12) cancers of the small intestine, including adenocarcinoma, lymphoma, carcinoid tumors, Kaposi's sarcoma, leiomyoma, hemangioma, lipoma, neurofibroma, and fibroma; (H) cancers of the urinary system, including: (1) cancers of the kidney, including renal cell carcinoma, carcinoma of Bellini's collecting ducts, adenocarcinoma, papillary carcinoma, tubular carcinoma, granular cell carcinoma, clear cell carcinoma (hypernephroma), sarcoma of the kidney, and nephroblastoma; (2) cancers of the renal pelvis and ureter, including transitional cell carcinoma, papillary transitional cell carcinoma, squamous cell carcinoma, and adenocarcinoma; (3) cancers of the urethra, including transitional cell carcinoma, squamous cell carcinoma, and adenocarcinoma; and (4) cancers of the urinary bladder, including carcinoma in situ, transitional urothelial cell carcinoma, papillary transitional cell carcinoma, squamous cell carcinoma, adenocarcinoma, undifferentiated; (I) cancers of muscle, bone, and soft tissue, including: (1) cancers of bone, including: (a) bone-forming: osteosarcoma; (b) cartilage-forming: chondrosarcoma and mesenchymal chondrosarcoma; (c) diant cell tumor, malignant; (d) Ewing's sarcoma; (e) vascular tumors: hemangioendothelioma, hemangiopericytoma, and angiosarcoma; (f) connective tissue tumors: fibrosarcoma, liposarcoma, malignant mesenchymoma, and undifferentiated sarcoma; and (g) other tumors: chordoma and adamantinoma of long bones; (2) cancers of soft tissues, including: alveolar soft-part sarcoma, angiosarcoma, epithelioid sarcoma, extraskeletal chondrosarcoma, fibrosarcoma, leiomyosarcoma, liposarcoma, malignant fibrous histiocytoma, malignant hemangiopericytoma, malignant mesenchymoma, malignant schwannoma, rhabdomyosarcoma, synovial sarcoma, and sarcoma (NOS); (3) cancers of the nervous system, including cancers of the skull (osteoma, hemangioma, granuloma, xanthoma, osteitis deformans), cancers of the meninges (meningioma, meningiosarcoma, gliomatosis), cancers of the brain (astrocytoma, medulloblastoma, glioma, ependymoma, germinoma (pilealoma), glioblastoma multiform, oligodendroglioma, schwannoma, retinoblastoma, congenital tumors), and cancers of the spinal cord neurofibroma, meningioma, glioma, sarcoma); (4) hematologic cancers, including myeloid leukemia (acute and chronic), acute lymphloblastic leukemia, chronic lymphocytic leukemia, myeloproliferative diseases, multiple myeloma; myelodysplastic syndrome), Hodgkin's disease, and non-Hodgkin's lymphoma (malignant lymphoma); (5) cancers of the endocrine system, including: (a) cancers of the thyroid gland, including papillary carcinoma (including those with follicular foci), follicular carcinoma, medullary carcinoma, and undifferentiated (anaplastic) carcinoma; and (b) neuroblastomas, including sympathicoblastoma, sympathicogonioma, malignant ganglioneuroma, gangliosympathicoblastoma, and ganglioneuroma; (6) cancers of the skin, including squamous cell carcinoma, spindle cell variant of squamous cell carcinoma, basal cell carcinoma, adenocarcinoma developing from sweat or sebaceous gland, and malignant melanoma; (7) cancers of the eye, including: (a) cancers of the conjunctiva, including carcinoma of the conjunctiva; (b) cancers of the eyelid, including basal cell carcinoma, squamous cell carcinoma, melanoma of the eyelid, and sebaceous cell carcinoma; (c) cancers of the lacrimal gland, including adenocarcinoma, adenoid cystic carcinoma, carcinoma in pleomorphic adenoma, mucoepidermoid carcinoma, and squamous cell carcinoma; (d) cancers of the uvea, including spindle cell melanoma, mixed cell melanoma, and epithelioid cell melanoma; (e) cancers of the orbit, including sarcoma of the orbit, soft tissue tumor, and sarcoma of bone; and (f) retinoblastoma.

Examples of nontumor hyperproliferative disorders include but are not limited to myelodysplastic disorders; cervical carcinoma-in-situ; familial intestinal polyposes such as Gardner syndrome; oral leukoplakias; histiocytoses; keloids; hemangiomas; inflammatory arthritis; hyperkeratoses and papulosquamous eruptions including arthritis-related eruptions. Also included are viral induced hyperproliferative diseases such as warts and EBV induced disease (i.e., infectious mononucleosis), scar formation, blood vessel proliferative disorders such as restenosis, atherosclerosis, instent stenosis, vascular graft restenosis, etc.; fibrotic disorders; psoriasis; glomerular nephritis; macular degenerative disorders; benign growth disorders such as prostate enlargement and lipomas; autoimmune disorders and the like.

Compositions according to the present invention can also be administered for the treatment of cardiac dysrhythmias, including but not limited to the Wolff-Parkinson-White syndrome and atrioventricular nodal reentrant tachycardia ventricular tachycardia (VT), atrial tachycardias, atrial flutter and atrial fibrillation supraventricular tachycardias.

Compositions according to the present invention can also be administered for the treatment of endometriosis, uterine fibroid (uterine leiomyomata) menorrhagia, cervical erosion, cervical polyp, and related conditions.

Compositions according to the present invention can also be administered for the treatment of the defects or disorders of intervertebral discs including but not limited to annular fissures, fragmentation of the nucleus pulposus, contained herniation (a herniated intervertebral disc), and degenerative intervertebral discs.

Compositions according to the present invention can also be administered for the treatment of additional diseases or conditions, including, but not limited to, Alzheimer's disease, osteoporosis, sleep apnea, erectile dysfunction, McArdle disease, and carbohydrate metabolism disorders.

Compositions according to the present invention can also be administered for reducing aging or fatigue. As used herein, the term “reducing aging” refers to lessening, ameliorating, or relieving the deleterious effects of aging (e.g., low vigor, memory loss, weakened vision or hearing, and joint pain) in a subject. As used herein, the term “reducing fatigue” refers to lessening, ameliorating, or relieving one or more of the symptoms of fatigue (low energy, poor endurance, and attention deficits) in a subject.

The subject to be treated can be a human patient or a socially or economically important animal, including, but not limited to, a dog, a cat, a horse, a cow, a goat, a sheep, or a pig. Compositions according to the present invention can be formulated for treatment of non-human mammalian species such as, but not limited to, those described above and can be used in veterinary medicine. Methods according to the present invention are not limited to the treatment of humans and can be adapted for use in veterinary medicine.

The compositions described above can be preliminarily screened for their efficacy in treating above-described diseases by an in vitro assay and then confirmed by animal experiments (See Examples 1-4 below) and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All of the publications cited herein are incorporated by reference in their entirety.

### Example 1

### In Vivo Assays on Anti-Obesity Effects

Each of 120 eight-week old Sprague-Dawley (SD) female rats and 100 eight-week old SD male rats was fed with an unlimited amount of food for 14 days. The food intake and weight change of each rat were measured daily. The food conversion rate of each rat was then calculated using the following equation:

1R=100×ΔW/Ft %

In this equation, R refers to the food conversion rate, ΔW refers to the weight change, and Ft refers to daily food intake. 88 female rats and 77 male rats were then selected and assigned to 11 groups, each group having 8 female rats and 7 male rats. Each of the following 10 test compositions was dissolved in a 10% glucose aqueous solution and was administered subcutaneously to a group of rats daily for 28 days: (1) metformin chloride (hereinafter referred to as metformin) 15 mg/kg, (2) a serotonin creatinine sulfate complex (hereinafter referred to as serotonin) 0.25 mg/kg, (3) aspirin 4 mg/kg, (4) serotonin 0.25 mg/kg+aspirin 4 mg/kg, (5) metformin 15 mg/kg+aspirin 4 mg/kg, (6) metformin 15 mg/kg+serotonin 0.25 mg/kg, (7) metformin 5 mg/kg+aspirin 4 mg/kg+serotonin 0.25 mg/kg, (8) metformin 15 mg/kg+aspirin 4 mg/kg+serotonin 0.25 mg/kg, (9) metformin 45 mg/kg+aspirin 4 mg/kg+serotonin 0.25 mg/kg, and (10) sibutramine 2 mg/kg. The rats in the 11^{th} group were not administered with any drug and were used as a control group. The results show that rats administered with a combination of metformin, aspirin, and serotonin gained less weight than rats administered with each ingredient alone or any combination of two ingredients. Further, the average weight gain of the rats decreased as the daily dosage of metformin increased.

The total food intakes over 28 days were measured for all groups. The results show that the total food intakes of groups (1)-(10) were substantially the same that of control group (11). In other words, the test compositions did not significantly affect the appetite of the rats.

The food conversion rates were calculated for all groups. The results show that rats administered with a combination of metformin, aspirin, and serotonin could have a much lower food conversion rate than rats administered with each ingredient alone or any combination of two ingredients.

### Example 2

### In Vivo Assays on Antihypertensive Effects

60 SD male rats (90-110 g) were provided by Guang Dong Medical Laboratory Animal Center (FuoShan, Guang Dong, China). After each rat was anesthetized, a U-shaped silver clamp with an inner diameter of 0.2-0.25 mm was used to narrow kidney artery. 40 rats with good recovery two weeks after the surgery were selected and assigned to 5 groups, each group having 8 rats. Each of the following 4 test compositions was dissolved in a 10% glucose aqueous solution and was administered to a group of rats daily for 9 weeks: (1) metformin 45 mg/kg+aspirin 4 mg/kg+serotonin 0.25 mg/kg, (2) metformin 15 mg/kg+aspirin 4 mg/kg+serotonin 0.25 mg/kg, (3) metformin 5 mg/kg+aspirin 4 mg/kg+serotonin 0.25 mg/kg, and (4) nitedipine 2 mg/kg. The rats in the 5^{th} group were administered with a 10% glucose aqueous solution only and were used as a control group. The test compositions were administered subcutaneously except for nitedipine, which was administered by gastric perfusion. The tail arterial pressure of each rat was measured at the end of the 5^{th} week and the 9^{th} week.

The results show that the blood pressure of the rats in group (1) at the end of the 5^{th} and 9^{th} weeks were significantly lowered than that of the rats in the control group (i.e., group (5)) and the group in which the rats were fed with nitedipine (i.e., group (4)).

### Example 3

### In Vivo Assays on Acute Antihypertensive Effects

Renovascular hypertensive rats were prepared as follows: A male SD rat (90-110 g) was anesthetized with pentobarbitol sodium (45 mg/kg). A U-shaped silver clamp with an inner diameter of 0.18 mm was used to narrow kidney artery. The blood pressure of the rat increased significantly after 3-6 weeks and stabilized after about 8 weeks. The rats having a systolic pressure of between 180-240 mmHg were used in the following steps.

The rats prepared above were assigned to 4 groups. Each of the following 3 test compositions were dissolved in a 10% glucose aqueous solution: (1) metformin 45 mg/kg+aspirin 4 mg/kg+serotonin 0.25 mg/kg, (2) metformin 15 mg/kg+aspirin 4 mg/kg+serotonin 0.25 mg/kg, and (3) metformin 5 mg/kg+aspirin 4 mg/kg+serotonin 0.25 mg/kg. The rats in the 4^{th} group were administered with a 10% glucose solution only and were used as a control group. Each rat was then anesthetized with pentobarbital sodium (45 mg/kg) and affixed to a board. A tube was inserted into trachea to maintain the breathing of the rat. Another tube was then inserted to the neck artery to measure the blood pressure. The blood pressure was measured by using a BL-420E biological signal collecting and processing system. When the neck artery blood pressure of the rat was stabilized, a test composition or the 10% glucose aqueous solution was administered subcutaneously in the abdomen section. The neck artery blood pressure was measured at 15, 30, 45, 60, 90, 120, 150, 180, 210, and 240 minutes after administration.

The results show that the neck artery blood pressure of the rats in groups (1) and (2) started to decrease at 15 minutes and reached the lowest levels at 120-150 minutes. The average neck artery blood pressure values were lowered as much as 29.7±5.2 mm Hg and 20.3±2.9 mm Hg, respectively, compared to that measured before administration of the test composition. The neck artery blood pressure did not return to the level before administration of the test composition even after 4 hours. The results also show that the test composition did not significantly affect the heart rate of the rats.

### Example 4

### In Vivo Assay on Effects of Lowering Blood Glucose Levels

Male Sprague-Dawley (SD) rats (180-210 g) were intraperitoneally injected with streptozotocin (50 mg/kg) to induce type 2 diabetes. Rats having blood glucose levels higher than 17 mmol/L after the injection were assigned randomly to five groups, each including 10 rats. The rats in each of the five groups were then treated with the three test compositions described in Example 3 above, i.e., metformin 45 mg/kg+aspirin 4 mg/kg+serotonin 0.2 mg/kg (high dose), metformin 15 mg/kg+aspirin 4 mg/kg+serotonin 0.2 mg/kg (medium dose), and metformin 5 mg/kg+aspirin 4 mg/kg+serotonin 0.2 mg/kg (low dose); metformin alone at the dosage of 0.135 g/kg (metformin); and a vehicle control (control). 10 normal male SD rats, serving as normal controls, were subjected to the same treatment.

The blood glucose level of each treated rat was measured before treatment and 3-hour, 6-hour, 3-day, 7-day, 14-day, and 21-day after treatment. Results thus obtained demonstrate that the three test compositions significantly lowered the blood glucose levels in the type 2 diabetic rats.

### Example 5

### Oral In Vivo Anti-Cancer Effects

In a first experiment, 20 KM female mice were used. The animals were randomly grouped into 10 mice per group after the body weight of the mice was measured in fasting condition. All mice were dosed appropriately by gavage two days before inoculation, TID, for 18 days continuously. For a cancer model, each mouse was inoculated with about 2×10⁶ H22 carcinoma cells at the back side of the right groin. The results are shown in Table 1 (n=10 per group). In conclusion, oral administration of APM (170 mg/kg metformin+400 mg/kg celecoxib+12 mg/kg melatonin) significantly inhibited the growth of subcutaneous inoculated H22 tumor cells in KM mice.

TABLE 1
(n = 10 per group)

| Group | Tumor Weight, g | Inhibition Rate, % | p-Value |
|---|---|---|---|
| 170 mg/kg | 0.34 ± 0.28** | 60.1 | 0.006 |
| metformin + 400 | | | |
| mg/kg celecoxib + | | | |
| 12 mg/kg melatonin | | | |
| 10% G.S. (control) | 0.84 ± 0.43 | | |

### Example 5

### Oral In Vivo Anti-Cancer Effects

In a second experiment, 16 KM female mice were used. The animals were randomly grouped into 8 mice per group after the body weight of the mice was measured in fasting condition. All mice were dosed appropriately by gavage two days before inoculation, TID, for 19 days continuously. For a cancer model, each mouse was inoculated with about 2×10⁶ H22 carcinoma cells at the back side of the right groin. The results are shown in Table 2 (n=8 per group). In conclusion, oral administration of ABM (170 mg/kg metformin+800 mg/kg aspirin+12 mg/kg melatonin) significantly inhibited the growth of subcutaneously inoculated H22 tumor cells in KM mice, with a 38.9% inhibition rate

TABLE 2
(n = 8 per group)

| Group | Tumor Weight, g | Inhibition Rate, % | p-Value |
|---|---|---|---|
| 170 mg/kg | 0.74 ± 0.47** | 38.9 | 0.044 |
| metformin + 800 | | | |
| mg/kg aspirin + 12 | | | |
| mg/kg melatonin | | | |
| 10% G.S. (control) | 1.20 ± 0.37 | | |

### Example 6

### Weight Loss Effects

Male SD rats (60 rats) were used. The rats were randomized into 15 rats per group based on their body weight. The designated drug combination was administered on the next day, TID, continuously for 5 weeks. Fasting body weight of the rats was measured; at the end of the experiment, the superior mesenteric, kidney, and epididymis fat depots were dissected and weighed. Data analysis was performed using the SPSS software package with one-way ANOVA. The drug was administered continuously for 5 weeks; the weight gain in low dose, mid dose and high dose ABM groups are 149.7 g, 140.5 g, and 125.4 g respectively, which are less than the control group (156.9 g). Moreover, the differences between control group and both the mid and high dose groups are significant. The average fat contents of low dose, mid dose, and high dose ABM were 16.1 g, 14.8 g, and 14.4 g respectively. In comparison to the control group (17.9 g), the fat contents of mid and high dose groups had decreased significantly with obvious differences. The results are shown in Table 3. In conclusion, 5 weeks of ABM administration continuously with the mid or high dose had significantly inhibited weight gain and had decreased body fat of normal rats.

TABLE 3
Weight Gain Effect of ABM in SD Rats (n = 15 per group)

| Group | Pre-dose (g) | Post-dose (g) | Δ Body weight (g) | Fat contents (g) |
|---|---|---|---|---|
| Metformin 17 | 253.5 ± 6.3 | 403.1 ± 19.5 | 149.7 ± 17.5 | 16.1 ± 3.1 |
| mg/kg + aspirin | | | | |
| 1 mg/kg + | | | | |
| melatonin 0.12 | | | | |
| mg/kg | | | | |
| Metformin 85 | 252.7 ± 5.3 | 393.1 ± 12.4** | 140.5 ± 12.7** | 14.8 ± 4.6* |
| mg/kg + aspirin | | | | |
| 5 mg/kg + | | | | |
| melatonin 0.6 | | | | |
| mg/kg | | | | |
| Metformin 170 | 252.6 ± 6.1 | 377.9 ± 21.1** | 125.4 ± 20.3** | 14.4 ± 3.3** |
| mg/kg + aspirin | | | | |
| 10 mg/kg + | | | | |
| melatonin 1.2 | | | | |
| mg/kg | | | | |
| 10 G.S. | 253.4 ± 6.9 | 410.3 ± 12.0 | 156.9 ± 12.9 | 17.9 ± 3.0 |
| (control) | | | | |

(*p < 0.05, **p < 0.01 versus G.S.)

### Example 7

### Anticancer Effects of Compositions Including Butyrate

In a first experiment, 20 KM female mice were used. The animals were randomly grouped into 10 mice per group after the body weight of the mice was measured in fasting condition. All mice were dosed appropriately by gavage two days before inoculation, TID, for 16 days continuously. For a cancer model, each mouse was inoculated with about 2×10⁶ H22 ascites carcinoma cells at the back side of the right groin. The results are shown in Table 4. In conclusion, continuous oral administration of JPM (12750 mg/kg butyrate+400 mg/kg celecoxib+80 mg/kg melatonin) for 16 days significantly inhibited the growth of subcutaneously inoculated H22 tumor cells in KM mice.

In a second experiment, 20 KM female mice were used. The animals were randomly grouped into 10 mice per group after the body weight of the mice was measured in fasting condition. All mice were dosed appropriately by gavage two days before inoculation, TID, for 16 days continuously. For a cancer model, each mouse was inoculated with about 2×10⁶ H22 ascites carcinoma cells at the back side of the right groin. The results are shown in Table 5. In conclusion, continuous oral administration of JBM (12750 mg/kg butyrate+800 mg/kg aspirin+80 mg/kg melatonin) 16 days significantly inhibited the growth of subcutaneously inoculated H22 tumor cells in KM mice.

TABLE 4
(n = 10 per group)

| Group | Tumor Weight, g | Inhibition Rate, % | p-Value |
|---|---|---|---|
| 12750 mg/kg | 0.41 ± 0.24 | 69.0 | 0.0084 |
| butyrate + 400 | | | |
| mg/kg celecoxib + | | | |
| 80 mg/kg melatonin | | | |
| 10% G.S. (control) | 1.31 ± 0.81 | | |

TABLE 5
(n = 10 per group)

| Group | Tumor Weight, g | Inhibition Rate, % | p-Value |
|---|---|---|---|
| 12750 mg/kg | 0.55 ± 0.36 | 53.7 | 0.010974 |
| butyrate + 800 | | | |
| mg/kg aspirin + 80 | | | |
| mg/kg melatonin | | | |
| 10% G.S. (control) | 1.19 ± 0.56 | | |

### ADVANTAGES OF THE PRESENT INVENTION AND OTHER EMBODIMENTS

Compositions and methods according to the present invention are effective in treating a number of diseases and conditions, including metabolic syndrome and diseases and conditions associated with metabolic syndrome, hyperproliferative diseases including cancer, AIDS, Parkinson's disease, polycystic ovarian syndrome, Alzheimer's disease, osteoporosis, sleep apnea, erectile dysfunction, McArdle disease, and carbohydrate metabolism disorders, cardiac dysrhythmias; endometriosis, uterine fibroid (uterine leiomyomata) menorrhagia, cervical erosion, cervical polyp, and related conditions, defects or disorders of intervertebral discs. Compositions and methods according to the present invention are well tolerated, produce few if any side effects, and can be used together with other known pharmaceutically active compounds and compositions for treating these conditions.

Compositions and methods according to the present invention possess industrial applicability as compositions and methods for the preparation of a medicament to treat the diseases and conditions described above.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims. When claims are written in a form employing the transitional phrase “comprising,” the recitation of “comprising” also encompasses any and all embodiments described by claims in which the transitional phrase is “consisting essentially of” or “consisting of,” and, should it prove advantageous to do so, the transitional phrases “consisting essentially of” or “consisting of” could be used in place of “consisting of.”

1. A composition comprising:
(a) an effective amount of a first agent selected from the group consisting of an oxidative phosphorylation inhibitor, an ionophore, and an adenosine 5′-monophosphate-activated protein kinase (AMPK) activator;
(b) an effective amount of a second agent that possesses anti-inflammatory activity; and
(c) an effective amount of a third agent that possesses or maintains serotonin activity.

2.-6. (canceled)

7. The composition of claim 1 wherein the first agent is an adenosine 5′-monophosphate-activated protein kinase (AMPK) activator.

8. The composition of claim 7 wherein the adenosine 5′-monophosphate-activated protein kinase (AMPK) activator is selected from the group consisting of: (1) metformin; (2) phenformin; (3) buformin; (4) AICAR; (5) a thienopyridone; (6) resveratrol; (7) nootkatone; (8) thiazole; (9) adiponectin; (10) 2-deoxyglucose; (11) AAPDs; (12) adiponectin variant polypeptides; (13) catechins; (14) trans-10, cis-12 conjugated linoleic acid; (15) a corydaline-related compound selected from the group consisting of corydaline, corlumidin, (+)-corlumidin, corypalmine, 14R—H-corypalmine, tetrahydropalmatine, 14R-(+)-tetrahydropalmatine, 14R,13S-(+)-corydaline, bicuculline, d-(+) -bicuculline, egenine, and +-egenine; (16) a dithiolethione; (17) an inhibitor or antagonist of DNA-dependent protein kinase catalytic subunit (DNA-PKcs); (18) a small interfering RNA (siRNA) that can inhibit the expression and/or translation of DNA-PKcs; (19) a fibrate selected from the group consisting of bezafibrate, ciprofibrate, fenofibrate, clofibrate, and gemfibrozil; (20) GW2974 (N4-(1-benzyl-1H-indazol-5-yl)-N6,N6-dimethyl-pyrido-[3,4-d]-pyrimidine-4,6-diamine); (21) honokiol; (22) leptin; (23) LKB1 (serine/threonine kinase 11); (24) obovatol (4′,5-diallyl-2,3-dihydroxybiphenyl ether); (25) a thiazolidinedione selected from the group consisting of pioglitazone and related thiazolidinediones, including rosiglitazone and rosiglitazone maleate; (26) a variant adiponectin peptide having one or more mutations at amino acid positions 109-229 of wild-type adiponectin and having at least threefold increased solubility when compared to wild-type adiponectin; (27) a butyrate compound selected from a butyrate salt and a butyrate ester; and (28) a quinoxalinedione derivative; and the salts, solvates, analogues, congeners, bioisosteres, hydrolysis products, metabolites, precursors, and prodrugs thereof.

9. The composition of claim 8 wherein the adenosine 5′-monophosphate-activated protein kinase (AMPK) activator is selected from the group consisting of metformin, phenformin, buformin, and the salts thereof, and a butyrate compound selected from the group consisting of a butyrate salt and a butyrate ester.

10. (canceled)

11. The composition of claim 1 wherein the second agent that possesses anti-inflammatory activity is selected from the group consisting of a steroidal anti-inflammatory drug and a non-steroidal anti-inflammatory drug.

12. (canceled)

13. The composition of claim 11 wherein the second agent that possesses anti-inflammatory activity is a steroidal anti-inflammatory drug and wherein the steroidal anti-inflammatory drug is selected from the group consisting of: (1) hydrocortisone; (2) cortisone; (3) beclomethasone; (4) betamethasone; (5) dexamethasone; (6) prednisone; (7) methylprednisolone; (8) triamcinolone; (9) fluocinolone; (10) fludrocortisone; (11) hyaluronic acid 6-methylprednisolone ester; (12) rimexolone; (13) deflazacort, (14) prednisolone; (15) ORG6632 (21-chloro-9α-11β-hydroxy-16α,17α-dimethylpregna-1,4-diene-3,20-dione); (16) 21-acetoxypregnenolone; (17) alclometasone; (18) algestone; (19) amcinonide; (20) azulfidine; (21) budesonide; (22) chloroprednisone; (23) clobetasol; (24) clocortolone; (25) cloprednol; (26) corticosterone; (27) desonide; (28) desoximetasone; (29) desoxycorticosterone; (30) diflorasone; (31) difluprednate; (32) enoxolone; (33) fluazacort; (34) flucloronide; (35) flumethasone; (36) flunisolide; (37) fluocortolone; (38) fluorometholone; (39) fluprednidene; (40) fluprednisolone; (41) fluticasone; (42) halcinonide; (43) halobetasol; (44) halometasone; (45) hydrocortamate; (46) medrysone; (47) meprednisone; (48) mometasone; (49) paramethasone; (50) prednicarbate; (51) prednival; (52) prednylidene; (53) tixocortol; (54) clobetasone; (55) cortivazol; (56) diflucortolone; (57) fluocinolone; (58) fluocortin; (59) fluperolone; (60) formocortal; (61) halopredone; (62) mazipredone; (63) 6α,9α-difluoro-17α-[(2-furanylcarbonyl)oxy]-11β-hydroxy-16α-methyl-3-oxoandrosta-1,4-diene-17β-carbothioic acid S-fluoromethyl ester; (64) 6α,9α-difluoro-11β-hydroxy-16α-methyl-3-oxo-17α-propionyloxy-androsta-1,4-diene-17β-carbothioic acid S-(2-oxo-tetrahydrofuran-3S-yl)ester; (65) rofleponide; (66) ciclesonide; (67) butixocort; (68) RPR-106541 (20R-16α,17α-[butylidenebis(oxy)]-6α,9α-difluoro-11β-hydroxy-17β-(methylthio)androsta-4-en-3-one); (69) ST-126 (9-Fluoro-11β,17,21-trihydroxy-16α-methyl-1,4-pregnadiene-3,20-dione 21-cyclohexanecarboxylate cyclopropanecarboxylate); (70) flurandrenolide; (71) 9α-fluoro-11β,17α-dihydroxy-21-methoxy-16α-methylpregna-1,4-diene-3,20-dione; (72) 9α-fluoro-11β,17α-dihydroxy-21-benzyl oxy-16α-methyl pregna-1,4-diene-3,20-dione; (73) 9α-fluoro-11β,17α-dihydroxy-21-(2-methoxyethoxy)methoxy-16α-methylpregna-1,4-diene-3,20-dione; (74) 9α-fluoro-11β,17α-dihydroxy-21-(2-hydroxylethoxy)-16α-methylpregna-1,4-diene-3,20-dione; (75) 9α-fluoro-11β,17α-dihydroxy-21-(methylthiomethoxy)-16α-methylpregna-1,4-diene-3,20-dione (76) 9α-fluoro-11β,17α-dihydroxy-21-(methoxy)methoxy-16α-methylpregna-1,4-diene-3,20-dione; (77) 9α-fluoro-11β,17α-dihydroxy-Δ₂₀-ethoxy-21-ethoxy-16α-methylpregna-1,4-diene-3,20-dione; (78) 9α-fluoro-11β,17α-dihydroxy-21-ethoxy-16α-methylpregna-1,4-diene-3,20-dione; (79) 9α-fluoro-11β,17α-dihydroxy-21-allyloxy-16α-methylpregna-1,4-diene-3,20-dione; (80) 9α-fluoro-11β,17α-dihydroxy-21-cyclopropylmethoxy-16α-methylpregna-1,4-diene-3,20-dione; (81) 9α-fluoro-11β,17α-dihydroxy-21-allyl-21-allyloxy-16α-methyl-1,4-diene-3,20-dione; (82) 9α-fluoro-11β,17α-hydroxy-21-isopropyloxy-16α-methylpregna-1,4-diene-3,20-dione; (83) 9α-fluoro-11β-propionoxy-17α-hydroxy-21-methoxy-16α-methyl pregna-3,20-dione; and (84) 9α-fluoro-11β-17α-diacetoxy-21-methoxy-16α-methylpregna-1,4-diene-3,20-dione; and the esters, acetonides, benetonides, furetonides, salts, solvates, analogues, congeners, bioisosteres, hydrolysis products, metabolites, precursors, and prodrugs thereof.

14. (canceled)

15. The composition of claim 11 wherein the second agent that possesses anti-inflammatory activity is a non-steroidal anti-inflammatory drug and wherein the non-steroidal anti-inflammatory drug is selected from the group consisting of: (1) A183827; (2) ABT963; (3) aceclofenac; (4) acemetacin; (5) acetaminophen; (6) acetylsalicylic acid; (7) ACP; (8) actarit; (9) AHR10037; (10) AHR15010; (11) alclofenac; (12) alminoprofen; (13) amfenac; (14) ampiroxicam (15) amtolmetin guacil; (16) apazone; (17) araprofen; (18) atliprofen methyl ester; (19) AU8001; (20) azapropazone; (21) bendazac; (22) benoxaprofen; (23) benzydamine; (24) benzydamine flufenamate; (25) bermoprofen; (26) benzpiperylon; (27) BF388; (28) BF389; (29) BIRL790; (30) BMS347070; (31) bromfenac; (32) bucloxic acid; (33) bumadizone; (34) butibufen; (35) BW4C; (36) BW755C; (37) C53; (38) C73; (39) C85; (40) carprofen; (41) CBS1108; (42) celecoxib; (43) CGS25997; (44) CHF2003; (45) chlorobiphenyl; (46) choline magnesium trisalicylate; (47) CHX108; (48) CI959; (49) cimicoxib; (50) cinmetacin; (51) cinnoxicam; (52) clidanac; (53) clofezone; (54) clonixin; (55) clopirac; (56) CLX1205; (57) COX-2 inhibitors; (58) CP331; (59) CS502; (60) CS706; (61) D1367; (62) darbufelone; (63) deracoxib; (64) dexibuprofen; (65) dexibuprofen lysine; (66) dexketoprofen; (67) DFP; (68) DFU; (69) diclofenac sodium; (70) diclofenac potassium; (71) diflunisal; (72) DP155; (73) DRF4367; (74) droxicam; (75) E5110; (76) E6080; (77) E6087; (78) eltenac; (79) enfenamic acid; (80) epirizole; (81) ER34122; (82) esflurbiprofen; (83) ethenzamide; (84) etodolac; (85) etofenamate; (86) etoricoxib; (87) F025; (88) FCE20696; (89) felbinac; (90) felbinac ethyl; (91) fenbufen; (92) fenclofenac; (93) fenclozic acid; (94) fenclozine; (95) fendosal; (96) fenoprofen; (97) fentiazac; (98) fepradinol; (99) feprazone; (100) filenadol; (101) flobufen; (102) florifenine; (103) flosulide; (104) flubichin methanesulfonate; (105) flufenamic acid; (106) flufenisal; (107) flunixin; (108) flunoxaprofen; (109) fluprofen; (110) fluproquazone; (111) flurbiprofen; (112) FPL62064; (113) FR111142; (114) FR122047; (115) FR123826; (116) FR140423; (117) FR188582; (118) FS205397; (119) furofenac; (120) GR80907; (121) GR129574A; (122) GR253035; (123) GW406381; (124) HAI105; (125) HAI106; (126) HCT2035; (127) HGP12; (128) 11N3392; (129) HP977; (130) HX0835; (131) HYAL AT2101; (132) ibufenac; (133) ibuprofen; (134) ibuproxam-beta-cyclodextrin; (135) icodulinum; (136) IDEA070; (137) iguratimod; (138) imrecoxib; (139) indomethacin; (140) indoprofen; (141) IP751; (142) IRA378; (143) isofezolac; (144) isoxepac; (145) isoxicam; (146) IX207887; (147) KC764; (148) ketoprofen; (149) ketorolac; (150) L652343; (151) L745337; (152) L748731; (153) L752860; (154) L651392; (155) L663536; (156) L761066; (157) L768277; (158) L776967; (159) L783003; (160) L784520; (161) L791456; (162) L804600; (163) L818571; (164) LAS33815; (165) LAS34475; (166) licofelone; (167) LM4108; (168) lobuprofen; (169) lornoxicam; (170) lonazolac; (171) loxaprofen; (172) lumaricoxib; (173) LY221608; (174) LY269415; (175) mabuprofen; (176) meclofenamic acid; (177) meclofenamate sodium; (178) mefenamic acid; (179) meloxicam; (180) mercaptoethylguanidine; (181) mesaclazone; (182) mesoporphyrin; (183) metoxibutropate; (184) miroprofen; (185) mofebutazone; (186) mofezolac; (187) morazone; (188) MX 1094; (189) nabumetone; (190) naproxen sodium; (191) naproxen sodium/metoclopramide; (192) NCX1101; (193) NCX284; (194) NCX285; (195) NCX4016; (196) NCX4215; (197) NCX530; (198) nepafanac; (199) niflumic acid; (200) nimesulide; (201) nitric oxide-based NSAIDs; (202) nitrofenac; (203) nitroflurbiprofen; (204) nitronaproxen; (205) NS398; (206) ocimum sanctum oil; (207) olsalazine; (208) ONO3144); (209) orpanoxin; (210) oxaceprol; (211) oxaprozin; (212) oxindanac; (213) oxpinac; (214) oxycodone/ibuprofen; (215) oxyphenbutazone; (216) P10294; (217) P54; (218) P8892; (219) pamicogrel; (220) parcetasal; (221) parecoxib; (222) parsalmide; (223) PD138387; (224) PD145246; (225) PD164387; (226) pelubiprofen; (227) pemedolac; (228) phenylbutazone; (229) pirazolac; (230) piroxicam; (231) piroxicam beta-cyclodextrin; (232) piroxicam pivalate; (233) pirprofen; (234) pranoprofen; (235) prinomide; (236) proglumetacin; (237) resveratrol; (238) R-ketoprofen; (239) R-ketorolac; (240) Ro323555; (241) rofecoxib; (242) RP54745; (243) RP66364; (244) RU43526; (245) RU46057; (246) RU54808; (247) RWJ63556; (248) S19812; (249) S33516; (250) salicin; (251) salicylamide; (252) salicylsalicylic acid; (253) satigrel; (254) SC236; (255) SC57666; (256) SC58125; (257) SC58451; (258) SD8381; (259) seprilose; (260) SFPP; (261) SKF105809; (262) SKF86002; (263) sodium salicylate; (264) sudoxicam; (265) sulfasalazine; (266) sulindac; (267) suprofen; (268) SVT2016; (269) T3788; (270) TA60; (271) talmetacin; (272) talniflumate; (273) tazofelone; (274) tebufelone; (275) tenidap; (276) tenoxicam; (277) tepoxalin; (278) tiaprofenic acid; (279) tiaramide; (280) tilmacoxib; (281) tilnoprofen arbamel; (282) tinoridine; (283) tiopinac; (284) tioxaprofen; (285) tolfenamic acid; (286) tolmetin; (287) triflusal; (288) tropesin; (289) TY10222; (290) TY10246; (291) TY10474; (292) UR8962; (293) U91502; (294) ursolic acid; (295) valdecoxib; (296) WAY120739; (297) WY28342; (298) WY41770; (299) WY46135; (300) ximoprofen; (301) YS134; (302) zaltoprofen; (303) ZD2138; (304) zidometacin; (305) zomepirac; (306) AA961; (307) acetaminosalol; (308) AD1590; (309) AFP802; (310) aloxiprin; (311) amfenac sodium; (312) aminopropylon; (313) aminopyrine; (314) amoxiprin; (315) anirolac; (316) anitrazafen; (317) antrafenine; (318) 2-arylpropionic acids; (319) azulene sodium sulfonate; (320) baicalein; (321) bendazac lysinate; (322) benorylate; (323) biphenyl aspirin; (324) BPPC; (325) bromfenac sodium; (326) broperamole; (327) bufexamac; (328) bufezolac; (329) BW540C; (330) caffeic acid; (331) calcium acetylsalicylate; (332) Chinoin 127; (333) choline salicylate; (334) cicloprofen; (335) cinchophen; (336) cintazone; (337) cipamfylline; (338) clobuzarit; (339) clometacin; (340) clonixeril; (341) cloximate; (342) CN100; (343) 4-(4-cyclohexyl-2-methyloxazol-5-yl)-2-fluorobenzenesulfonamide; (344) cyclooxygenase-1 inhibitors; (345) delmetacin; (346) dexindoprofen; (347) diaryl-5-oxygenated-2-(5H)-furanone; (348) 2,4-dichlorobenoxaprofen; (349) difenpiramide; (350) diflumidone sodium; (351) 2-(3,5-difluorophenyl)-3-[4-(methylsulfonyl)phenyl]-2-cyclopenten-1-one); (352) diftalone; (353) dimethylisopropylazulene; (354) 5,5-dimethyl-3-isopropyloxy-4-(4′-methylsulfonylphenyl)-2(5H)-furanone; (355) dimethyl sulfoxide; (356) DKA9; (357) DUP697; (358) EB382; (359) eicosatriynoic acid; (360) emorfazone; (361) enolicam; (362) ethyleneglycol salicylate; (363) F1044; (364) fenamates; (365) fenamole; (366) fenbuprofen; (367) fenclorac; (368) fenflumizole; (369) fenoprofen calcium; (370) floctafenine; (371) flunixin meglumine; (372) flurbiprofen axetil; (373) fosfosal; (374) furcloprofen; (375) glafenine; (376) glucametacin; (377) GP53633; (378) 5(S)-1-1E1E; (379) 5-RETE lactone; (380) ibuprofen aluminum; (381) ibuprofen piconol; (382) ibuproxam; (383) imidazole salicylate; (384) indometacin farnesil; (385) indomethacin sodium trihydrate; (386) indoxole; (387) intrazole; (388) ITC1; (389) ITF182; (390) JTE522; (391) KB1043; (392) KC8973; (393) ketophenylbutazone; (394) ketorolac tromethamine; (395) KME4; (396) LA2851; (397) 5-lipoxygenase inhibitors; (398) lofemizole; (399) lonazolac calcium; (400) lotifazole; (401) lysine acetylsalicylate; (402) lysine clonixinate; (403) LU20884; (404) M7074; (405) magnesium salicylate; (406) mefenamic acid aluminum; (407) mesalamine; (408) metamizole sodium; (409) metazamide; (410) metiazinic acid; (411) 6-methoxy-2 naphthylacetic acid; (412) MG18311; (413) mixed PDE3/PDE4 inhibitors; (414) morniflumate; (415) morpholine salicylate; (416) MR714; (417) MR897; (418) N-acetyl-5-aminosalicylic acid; (419) 1-naphthyl salicylate; (420) N-[2-(cyclohexyloxy)-4-nitrophenyl]methanesulfonamide; (421) neocinchophen; (422) nictindole; (423) nifenazone; (424) 2-(2-nitroxy)-butyl 2-acetoxybenzoate; (425) 2-(2-nitroxymethyl)phenyl 2-acetoxybenzoate; (426) NO164; (427) NPPB; (428) N-(2-pyridyl)-2-methyl-4-cinnamoyloxy-2H-1,2-benzothiazine-3-carboxamido 1,1-dioxide; (429) o-(acetoxyphenyl)hept-2-ynyl sulfide (APHS); (430) olsalazine oxaceprol; (431) olsalazine sodium; (432) oxametacin; (433) oxapadol; (434) oxicams; (435) oxyphenthatrazone; (436) paranylene; (437) peroxisal; (438) peroxisal citrate; (439) phenazone; (440) phenidone; (441) phenyl O-acetylsalicylate; (442) pifoxime; (443) piketoprofen; (444) pimeprofen; (445) piprofen; (446) piroxicam cinnamate; (447) proglumetacin maleate; (448) propyphenazone; (449) proquazone; (450) protizinic acid; (451) QZ16; (452) R830; (453) R-enantiomers of acrylacetic acids; (454) R-enantiomers of arylpropionic acids; (455) R-enantiomers of thiazinecarboxamides; (456) RS2131; (457) RS57067; (458) RU16029; (459) salicylamide O-acetic acid; (460) SC560; (461) SCR152; (462) sermetacin; (463) sodium acetylsalicylate; (464) sodium thiosalicylate; (465) sulindac sulfide; (466) suxibutazone; (467) T614; (468) TAI901; (469) tesicam; (470) tetrydamine; (471) thromboxane inhibitors; (472) tiflamizole; (473) timegadine; (474) tinoridine hydrochloride; (475) tomoxiprol; (476) triethanolamine salicylate; (477) triflumidate; (478) trimethazone; (479) TVX960; (480) TVX2706; (481) TZI615; (482) U60257; (483) ufenamate; (484) vedaprofen; (485) WY23205; (486) xenbucin; and (487) zileuton; and the salts, solvates, analogues, congeners, bioisosteres, hydrolysis products, metabolites, precursors, and prodrugs thereof.

16. The composition of claim 15 wherein the non-steroidal anti-inflammatory drug is selected from the group consisting of acetylsalicylic acid and the salts thereof.

17. The composition of claim 15 wherein the non-steroidal anti-inflammatory drug is selected from the group consisting of alminoprofen, araprofen, atliprofen methyl ester, benoxaprofen, bermoprofen, butibufen, carprofen, dexibuprofen, dexibuprofen lysine, dexketoprofen, esflurbiprofen, fenbufen, fenoprofen, flobufen, flunoxaprofen, fluprofen, flurbiprofen, ibuprofen, indoprofen, ketoprofen, lobuprofen, loxaprofen, mabuprofen, miroprofen, MX1094, naproxen sodium, naproxen sodium/metoclopramide, nitroflurbiprofen, nitronaproxen, oxaprozin, oxycodone/ibuprofen, pelubiprofen, pirprofen, pranoprofen, R-ketoprofen, suprofen, tiaprofenic acid, tilnoprofen arbamel, tioxaprofen, ximoprofen, zaltoprofen, cicloprofen, dexindoprofen, 2,4-dichlorobenoxaprofen, fenbuprofen, fenoprofen calcium, flurbiprofen axetil, furcloprofen, ibuprofen aluminum, ibuprofen piconol, piketoprofen, pimeprofen, piprofen, and vedaprofen, and the salts thereof.

18. The composition of claim 15 wherein the non-steroidal anti-inflammatory drug is selected from the group consisting of aceclofenac, acemetacin, alclofenac, amfenac, bendazac, bromfenac, clidanac, clopirac, CP331, diclofenac sodium, diclofenac potassium, eltenac, etodolac, felbinac, felbinac ethyl, fenclofenac, furofenac, HCT6015, HYAL AT2101, ibufenac, indomethacin, isofezolac, ketorolac, LM4108, lonazolac, mofezolac, NCX284, NCX285, NCX530, nepafanac, nitrofenac, oxindanac, pemedolac, pirazolac, R-ketorolac, sulindac, talmetacin, tiopinac, zidometacin, zomepirac, amfenac sodium, anirolac, bendazac lysinate, bromfenac sodium, bufexamac, bufezolac, clometacin, delmetacin, fenclorac, glucametacin, indometacin farnesil, indomethacin sodium trihydrate, ketorolac tromethamine, lonazolac calcium, oxametacin, proglumetacin maleate, and sermetacin, and the salts thereof.

19. The composition of claim 15 wherein the non-steroidal anti-inflammatory drug is selected from the group consisting of ampiroxicam, cinnoxicam, isoxicam, lornoxicam, meloxicam, piroxicam, piroxicam beta-cyclodextrin, piroxicam pivalate, sudoxicam, tenoxicam, enolicam, piroxicam cinnamate, and tesicam, and the salts thereof.

20. The composition of claim 15 wherein the non-steroidal anti-inflammatory drug is selected from the group consisting of benzydamine flufenamate, enfenamic acid, etofenamate, flufenamic acid, meclofenamic acid, meclofenamate sodium, mefenamic acid, tolfenamic acid, mefenamic acid aluminum, and ufenamate, and the salts thereof.

21. (canceled)

22. The composition of claim 15 wherein the non-steroidal anti-inflammatory drug is acetaminophen or a salt thereof.

23. (canceled)

24. The composition of claim 15 wherein the non-steroidal anti-inflammatory drug is selected from the group consisting of celecoxib, cimicoxib, deracoxib, etoricoxib, imrecoxib, lumiracoxib, parecoxib, rofecoxib, tilmacoxib, and valdecoxib, and the salts thereof.

25. (canceled)

26. The composition of claim 15 wherein the non-steroidal anti-inflammatory drug is nimesulide or a salt thereof.

27. The composition of claim 1 wherein the third agent that possesses or maintains serotonin activity is selected from the group consisting of serotonin, a serotonergic compound, and a serotonin metabolite.

28. The composition of claim 27 wherein the third agent is serotonin or a salt thereof.

29. The compound of claim 27 wherein the third agent is selected from the group consisting of serotonin sulfate, a serotonin creatinine sulfate complex, and serotonin hydrochloride.

30. The composition of claim 27 wherein the third agent is a serotonergic compound selected from the group consisting of a serotonin transporter inhibitor, a serotonin receptor 1A antagonist, a serotonin receptor 2C modulator, a serotonin reuptake inhibitor, a serotonin and noradrenaline reuptake inhibitor, a serotonin dopamine antagonist, a monoamine reuptake inhibitor, a pyridazinone aldose reductase inhibitor, a stimulant of serotonin receptors, a stimulant of serotonin synthesis, and a serotonin agonist.

31. (canceled)

32. The composition of claim 27 wherein the third agent is a serotonin metabolite and wherein the serotonin metabolite is selected from the group consisting of 5-HIAA, melatonin, and the salts thereof.

33. The composition of claim 1 wherein the third agent is selected from the group consisting of: (1) paroxetine; (2) fluoxetine; (3) fenfluramine; (4) fluvoxamine; (5) sertraline; (6) imipramine; (7) BVT933; (8) DPCA37215; (9) IK264; (10) PNU22394 (6-methyl-1,2,3,4,5,6-hexahydro-azepino[4,5-b]indole); (11) WAY161503 (8,9-dichloro-2,3,4,4a-tetrahydro-1H-pyrazino[1,2-a]quinoxalin-5(6H)-one hydrochloride); (12) R-1065; (13) YM348 ((2S)-1-(7-ethyl-1H-furo[2,3-g]indazol-1-yl)propan-2-amine); (14) milnacipran; (15) citalopram; (16) desmethylsertraline (a metabolite of sertraline); (17) norfluoxetine; (18) desmethylcitalopram (a metabolite of citalopram); (19) escitalopram; (20) femoxetine; (21) ifoxetine; (22) cyanodothiepin; (23) litoxetine; (24) dapoxetine; (25) nefazodone; (26) cericlamine; (27) trazodone; (28) mirtazapine; (29) indalpine; (30) indeloxazine; (31) sibutramine; (32) zimeldine; (33) (+)-N-[1-[1-(4-chlorophenyl)cyclobutyl]-3-methylbutyl}-N-methylamine; (34) (+)-N-{1-[1-(4-chlorophenyl)cyclobutyl-3-methylbutyl}-N-methylamine; (35) (+)-1-[1-(4-chlorophenyl)cyclobutyl]-3-methylbutylamine; (36) (+)-N-{1-[1-(4-chlorophenyl)cyclobutyl]-3-methylbutyl}-N; (37) (+)-N-{1-[1-(4-chlorophenyl)cyclobutyl}-3-methylbutyl-N,N-dimethylamine)N-dimethylamine; (38) venlafaxine; (39) O-desmethylvenlafaxine (a metabolite of venlafaxine); (40) clomipramine; (41) desmethylclomipramine (a metabolite of clomipramine); (42) buspirone; (43) olanzapine; (44) ziprasidone; (45) ergoloid mesylates; (46) pergolide mesylate; (47) vitamin B1; (48) vitamin B3; (49) vitamin B6; (50) biotin; (51) S-adenosylmethionine; (52) folic acid; (53) folinic acid; (54) ascorbic acid; (55) magnesium; (56) coenzyme Q10; (57) piracetam; (58) (+)-2,5-dimethoxy-4-iodoamphetamine; (59) (+)-3,4-methylenedioxyamphetamine; (60) (+)-N-[2-[4-[2,3-dihydro-2-(hydroxymethyl)-1,4-benzodioxin-5-yl]1-piperazinyl]-4-fluorobenzamide hydrochloride; (61) (+)-norfenfluramine (a metabolite of fenfluramine); (62) (3β)-2,3-dihydrolysergene; (63) (3β)-2,3-dihydrolysergol; (64) (3β)-2,3-dihydro-methyllysergate; (65) (3β, 5β, 8β)-9,10-didehydro-2,3-dihydro-6-methyl-8-(2-pyridylthiomethyl)ergoline; (66) (3β, 5β, 8β)-9,10-didehydro-2,3-dihydro-6-methyl-8-(methylthiomethyl)ergoline; (67) (3β, 5β, 8β)-9,10-didehydro-2,3-dihydro-6-methyl-8-(phenylthiomethyl)ergoline; (68) (3β, 5β, 8β)-9,10-didehydro-2,3-dihydro-8-methyl-6-propylergoline; (69) 1-(4-bromo-2,5-dimethoxyphenyl)-2-aminopropane; (70) 1-(m-trifluoromethylphenyl)-piperazine; (71) 2-(4-(4-(2-pyrimidinyl)1-piperazinyl-propyl)-1,2-benzoisothiazol-3-(2H)-one 1,1-dioxide hydrochloride; (72) 2-methylserotonin; (73) 3β, 5β, 8β)-9,10-didehydro-2,3-dihydro-6-methylergoline-8-acetonitrile; (74) zolmitriptan; (75) 3 a,4,4a,6a,7,7a-hexahydro-2-[4-[4-(2-pyrimidinyl)-11-piperazinyl]butyl]-4,7-etheno-1H-cyclobutanoisoindole-1,3(2H)-dione dihydrochloride sesquihydrate; (76) 3-butyl-9,9-dimethyl-7-[4-[4-(2-methoxyphenyl) 1-piperazinyl]butyl]-3,7-diazabicyclo[3,2,1]nonane-2,4,6,8-tetraone; (77) 4,4-dimethyl-1-[4-[4-(2-pyrimidinyl)-1-piperazinyl]butyl]2,6-piperidinedione hydrochloride; (78) 5-hydroxy-L-tryptophan; (79) 5-methoxy-N,N-dimethyltryptamine; (80) 6-[[3-[4[o-methoxyphenyl]-1-piperazinyl]propyl]-amino]-1,3-dimethyluracil; (81) 8-[4-N-[4-(2-pyrimidinyl)-1-piperazinyl]-butyl]-8-azaspiro[4.5]-decane-7,9-dione hydrochloride; (82) 8-hydroxy-2-(di-n-propylamino)tetralin (8-OH-DPAT); (83) alniditan; (84) almotriptan; (85) 2-aminotetralin; (86) bifeprunox; (87) gepirone; (88) BW723C86 (1-[5(2-thienylmethoxy)-1H-3-indolyl[propan-2-amine hydrochloride); (89) cisapride; (90) dihydroergotamine; (91) D-lysergic acid diethylamide; (92) donitriptan; (93) eletriptan; (94) frovatriptan; (95) tegaserod; (96) ipsapirone; (97) L694247 (2-[5-[3-(4-methylsulphonylamino)benzyl-1,2,4-oxadiazol-5-yl]-1H-indol-3-yl]ethanamine); (98) cinitapride; (99) lesopitron; (100) MCPP (m-chlorophenylpiperazine); (101) methysergide; (102) metoclopramide; (103) MK-212 (6-chloro-2-(1-piperazinyl)pyrazine hydrochloride); (104) mosapride; (105) N,N-dimethyl-5-methoxytryptamine; (106) N,N-dimethyltryptamine; (107) N-[4-[4-(2-pyrimidinyl)-1-piperazinyl]butylbicyclo[2.2.1]heptane-2,3-di-oxo-carboximide; (108) naratriptan; (109) norcisapride; (110) phentermine; (111) quipazine; (112) prucalopride; (113) rauwolscine; (114) repinotan; (115) rizatriptan; (116) sumatriptan; (117) tandospirone; (118) 1-methyl-4-phenyl-1,2,3,6-tetrahydropyridine; (119) tiaspirone; (120) trifluoromethylphenylpiperazine; (121) L-tryptophan; (122) xaliproden; (123) yohimbine; (124) zacopride; (125) zalospirone (126) mianserin; (127) setiptiline; (128) adatanserin; (129) altanserin; (130) benanserin; (131) blonanserin; (132) butanserin; (133) cinanserin; (134) eplivanserin; (135) flibanserin (136) glemanserin; (137) iferanserin; (138) ketanserin; (139) lidanserin; (140) pelanserin; (141) pruvanserin; (142) ritanserin; (143) seganserin; (144) tropanserin; (145) iloperidone; (146) sertindole; (147) EMR-62218; (148) asenapine; (149) zotepine; (150) ocaperidone; (151) APD125; (152) AVE8488; (153) pimavanserin; (154) isocarboxazid; (155) phenelzine; (156) tranylcypromine; (157) amitriptyline; (158) clomipramine; (159) N-(1-(1-methylethyl)piperidin-4-yl)-N-((4-methylphenyl)methyl)-4-methoxyphenylacetamide; (160) N-(1-(2,2-dimethylethyppiperidin-4-yl)-N-((4-methylphenyl)methyl)-4-methoxyphenylacetamide; (161) N-(1-pentylpiperidin-4-yl)-N-((4-methylphenyl)methyl)-4-methoxyphenylacetamide; (162) N-(1-hexylpiperidin-4-yl)-N-((4-methylphenyl)methyl)-4-methoxyphenylacetamide; (163) N-(1-cyclohexylpiperidin-4-yl)-N-((4-methylphenyl)methyl)-4-methoxyphenylacetamide; (164) N-(1-cyclopentylpiperidin-4-yl)-N-((4-methylphenyl)methyl)-4-methoxyphenylacetamide, (165) N-(1-cyclobutylpiperidin-4-yl)-N-((4-methylphenyl)methyl)-4-methoxyphenylacetamide; (166) N-(1-cyclopropylpiperidin-4-yl)-N-((4-methylphenyl)methyl)-4-methoxyphenylacetamide; (167) N-(1-(cyclopentylmethyl)piperidin-4-yl)-N-((4-methylphenyl)methyl)-4-methoxyphenylacetamide; (168) N-(1-(cyclobutylmethyppiperidin-4-yl)-N-((4-methylphenyl)methyl)-4-methoxyphenylacetamide; (169) N-(1-(cyclopropylmethyppiperidin-4-yl)-N-((4-methylphenyl)methyl)-4-methoxyphenylacetamide; (170) N-(1-(2-hydroxyethyl)piperidin-4-yl)-N-((4-methylphenyl)methyl)-4-methoxyphenylacetamide; (171) N-(1-(3-hydroxypropyl)piperidin-4-yl)-N-((4-methylphenyl)methyl)-4-methoxyphenylacetamide; (172) N-((4-Methylphenyl)methyl)-N-(piperidin-4-yl)-N′-phenylmethylcarbamide; (173) N-((4-Methylphenyl)methyl)-N-(1-(2-methylpropyl)piperidin-4-yl)-N′-phenylmethylcarbamide; (174) N-(1-((2-Bromophenyl)methyl)piperidin-4-yl)-N-((4-methylphenyl)methyl)-N′-phenylmethylcarbamide; (175) N-(4-Hydroxy-3-methoxyphenyl)methyl)piperidin-4-yl)-N-((4-methylphenyl)methyl)-N′-phenylmethylcarbamide; (176) N-(1-((5-Ethylthien-2-yOmethyl)piperidin-4-yl)-N-((4-methylphenyl)methyl)-N′-phenylmethylcarbamide; (177) N-(1-(Imidazol-2-ylmethyl)piperidin-4-yl)-N-((4-methylphenyl)methyl)-N′-phenylmethylcarbamide; (178) N-(1-(Cyclohexylmethyl)piperidin-4-yl)-N-((4-methylphenyl)methyl)-N′-phenylmethylcarbamide; (179) N-(1-((4-Fluorophenyl)methyl)piperidin-4-yl)-N-((4-methylphenyl)methyl)-N′-phenylmethylcarbamide; (180) N-((4-Methylphenyl)methyl)-N-(piperidin-4-yl)-4-methoxyphenylacetamide; (181) N-((4-Methylphenyl)methyl)-N-(1-methylpiperidin-4-yl)-4-methoxyphenylacetamide; (182) N-(1-Ethylpiperidin-4-yl)-N-((4-methylphenyl)methyl)-4-methoxyphenylacetamide; (183) N-((4-Methylphenyl)methyl)-N-(1-propylpiperidin-4-yl)-4-methoxyphenylacetamide; (184) N-(1-Butylpiperidin-4-yl)-N-((4-methylphenyl)methyl)-4-methoxyphenylacetamide; (185) N-(1-(3,3-Dimethylbutyppiperidin-4-yl)-N-((4-methylphenyl)methyl)-4-methoxyphenylacetamide; (186) N-(1-(Cyclohexylmethyl)piperidin-4-yl)-N-((4-methylphenyl)methyl)-4-methoxyphenylacetamide; (187) N-((4-Methylphenyl)methyl)-N-(1-(2-methylpropyl)piperidin-4-yl)-4-methoxyphenylacetamide; (188) N-((4-Methylphenyl)methyl)-N-(1-((4-methylphenyl)methyl)piperidin-4-yl)-4-methoxyphenylacetamide; (189) N-(1-((4-Hydroxyphenyl)methyppiperidin-4-yl)-N-((4-methylphenyl)methyl)-4-methoxyphenylacetamide; (190) N-(1-((2-Hydroxyphenyl)methyl)piperidin-4-yl)-N-((4-methylphenyl)methyl)-4-methoxyphenylacetamide; (191) N-(3-Phenylpropyl)-N-(piperidin-4-yl)-4-methoxyphenylacetamide; (192) N-(2-Phenylethyl)-N-(piperidin-4-yl)-4-methoxyphenylacetamide; (193) N-((2-Methoxyphenyl)methyl)-N-(piperidin-4-yl)-4-methoxyphenylacetamide; (194) N-((2-Chlorophenyl)methyl)-N-(piperidin-4-yl)-4-methoxyphenylacetamide; (195) N-((3,4-Di-methoxyphenyl)methyl)-N-(piperidin-4-yl)-4-methoxyphenylacetamide; (196) N-((4-Fluorophenyl)methyl)-N-(piperidin-4-yl)-4-methoxyphenylacetamide; (197) N-((2,4-Di-chlorophenyl)methyl)-N-(piperidin-4-yl)-4-methoxyphenylacetamide; (198) N-((3-Methylphenyl)methyl)-N-(piperidin-4-yl)-4-methoxyphenylacetamide; (199) N-((3-Bromophenyl)methyl)-N-(piperidin-4-yl)-4-methoxyphenylacetamide; (200) N-(1-(Phenylmethyl)piperidin-4-yl)-N-(3-phenyl-2-propen-1-yl)-4-methoxyphenylacetamide; (201) N-((4-Methylphenyl)methyl)-N-(1-piperidin-4-yl)-phenylacetamide; (202) N-((4-Methylphenyl)methyl)-N-(1-piperidin-4-yl)-3-phenylpropionamide; (203) N-((4-Methylphenyl)methyl)-N-(1-piperidin-4-yl)-(phenylthio)acetamide; (204) N-((4-Methylphenyl)methyl)-N-(1-piperidin-4-yl)-phenoxyacetamide; (205) N-((4-Methylphenyl)methyl)-N-(1-piperidin-4-yl)-(4-chlorophenoxy)acetamide; (206) N-((4-Methylphenyl)methyl)-N-(1-piperidin-4-yl)-3-methoxyphenylacetamide; (207) N-((4-Methylphenyl)methyl)-N-(1-piperidin-4-yl)-4-fluorophenylacetamide; (208) N-((4-Methylphenyl)methyl)-N-(1-piperidin-4-yl)-2,5-di-methoxyphenylacetamide; (209) N-((4-Methylphenyl)methyl)-N-(1-piperidin-4-yl)-4-chlorophenylacetamide; (210) N-((4-Methylphenyl)methyl)-N-(1-(phenylmethyl)pyrrolidin-3-yl)-N′-phenylmethylcarbamide; (211) N-((4-Methylphenyl)methyl)-N-(1-(phenylmethyl)pyrrolidin-3-yl)-4-methoxyphenylacetamide; (212) 2-(4-methoxyphenyl)-N-((4-methylbenzyl)-N-(piperidin-4-yl)acetamide; (213) 2-(4-methoxyphenyl)-N-(4-methylbenzyl)-N-(1-methylpiperidin-4-yl)acetamide; (214) 2-(4-methoxyphenyl)-N-(4-methylbenzyl)-N-(1-ethylpiperidin-4-yl)acetamide; (215) 2-(4-methoxyphenyl)-N-(4-chlorbenzyl)-N-(1-ethylpiperidin-4-yl)acetamide; (216) 2-(4-methoxyphenyl)-N-(4-chlorbenzyl)-N-(1-isopropylpiperidin-4-yl)acetamide; (217) 2-(4-methoxyphenyl)-N-(4-chlorobenzyl)-N-(piperidin-4-yl)acetamide; (218) 2-(4-methoxyphenyl)-N-(4-chlorobenzyl)-N-(1-cyclopentylpiperidin-4-yl)acetamide; (219) 2-(4-methoxyphenyl)-N-(4-chlorbenzyl)-N-(1-isopropylpiperidin-4-yl)acetamide; (220) 2-(phenyl)-N-(4-trifluoromethylbenzyl)-N-(1-methylpiperidin-4-yl)acetamide; (221) 2-(4-fluorophenyl)-N-(4-trifluoromethylbenzyl)-N-(1-methylpiperidin-4-yl)acetamide; (222) 2-(4-Methoxyphenyl)-N-(4-trifluoromethylbenzyl)-N-(1-methylpiperidin-4-yl)acetamide; (223) 2-(4-Trifluoromethylphenyl)-N-(4-trifluoromethylbenzyl)-N-(1-methylpiperidin-4-yl)acetamide; (224) 2-(4-Fluorophenyl)-N-(4-fluorobenzyl)-N-(1-methylpiperidin-4-yl)acetamide; (225) 2-(4-Methoxyphenyl)-N-(4-fluorobenzyl)-N-(1-methylpiperidin-4-yl)acetamide; (226) 2-(phenyl)-N-(4-fluorobenzyl)-N-(1-methylpiperidin-4-yl)acetamide; (227) 2-(4-Trifluoromethylphenyl)-N-(4-fluorobenzyl)-N-(1-methylpiperidin-4-yl)acetamide; (228) 2-(4-trifluoromethylphenyl)-N-[4-(methoxycarbonyl)benzyl]-N-(1-methylpiperidin-4-yl)acetamide; (229) 2-Phenyl-N-[4-(methoxycarbonyl)benzyl]-N-(1-methylpiperidin-4-yl)acetamide; (230) 2-(4-Chlorophenyl)-N-[4-(methoxycarbonyl)benzyl]-N-(1-methylpiperidin-4-yl)acetamide; (231) 2-(4-Methoxyphenyl)-N-[4-(methoxycarbonyl)benzyl]-N-(1-methylpiperidin-4-yl)acetamide; (232) 2-(4-trifluoromethylphenyl)-N-[4-(methoxycarbonyl)benzyl]-N-(1-methylpiperidin-4-yl)acetamide; (233) 2-Phenyl-N-[4-(methoxycarbonyl)benzyl]-N-(1-methylpiperidin-4-yl)acetamide; (234) 2-(4-Chlorophenyl)-N-[4-(methoxycarbonyl)benzyl]-N-(1-methylpiperidin-4-yl)acetamide; (235) 2-(4-Methoxyphenyl)-N-[4-(methoxycarbonyl)benzyl]-N-(1-methylpiperidin-4-yl)acetamide; (236) 2-(4-methoxyphenyl)-N-(4-methylbenzyl)-N-[1-(4-chloromethyl-2-thiazolylmethyl)piperidin-4-yl]acetamide, (237) 2-(4 methoxyphenyl)-N-(4-methylbenzyl)-N-{1-[3-(1,3-dihydro-2H-benzimidazol-2-on-1-yepropyl]piperidin-4-yl}acetamide; (238) 2-(4-methoxyphenyl)-N-(2-4(fluorophenyl)ethyl)-N-(1-methylpiperidin-4-yl)acetamide; (239) 2-(4-methoxyphenyl)-N-[2-(2,5-dimethoxyphenyl)ethyl]-N-(1-methylpiperidin-4-yl)acetamide; (240) 2-(4-methoxyphenye-N-[2-(2,4-dichlorophenyl)ethyl]-N-(1-methylpiperidin-4-yl)acetamide; (241) 2-(4-methoxyphenyl)-N-[2-(3-chlorophenyl)ethyl]-N-(1-methylpiperidin-4-yl)acetamide; (242) 2-(4-methoxyphenyl)-N-[2-(4-methoxyphenyl)ethyl]-N-(1-methylpiperidin-4-yl)acetamide; (243) 2-(4-methoxyphenyl)-N-[2-(3-fluorophenyl)ethyl]-N-(1-methylpiperidin-4-yl)acetamide; (244) 2-(4-ethoxyphenyl)-N-[2-(4-fluorophenethyl]-N-(1-methylpiperidin-4-yl)acetamide; (245) 2-(4-ethoxyphenyl)-N-(4-fluorobenzyl)-N-(1-methylpiperidin-4-yl)acetamide; (246) 2-(4-methoxyphenyl)-N-(4-methylbenzyl)-N-{1-[2-(2-hydroxyethoxy)ethyl]piperidin-4-yl}acetamide; (247) 2-(4-methoxyphenyl)-N-(4-methylbenzyl)-N-[1-((2-chloro-5-thienyl)methyl)piperidin-4-yl]acetamide; (248) 2-(4-methoxyphenyl)-N-(4-methylbenzyl)-N-[1-(2-(imidazolidinon-1-yl)ethyl)piperidin-4-yl]acetamide; (249) 2-(4-methoxyphenyl)-N-(4-methylbenzyl)-N-{1-[2-(2,4(1H,3H)quinazolinedion-3-yl)ethyl]piperidin-4-yl}acetamide; (250) 2-(4-methoxyphenyl)-N-(4-methylbenzyl)-N-{1-[2-(1,3-dioxolan-2-yl)ethyl]piperidin-4-yl}acetamide; (251) 2-(4-methoxyphenyl)-N-(4-methylbenzyl)-N-{1-[2-(3-indolyl)ethyl]piperidin-4-yl}acetamide; (252) 2-(4-methoxyphenyl)-N-(4-methylbenzyl)-N-{1-[3-(1,2,4-triazol-1-yl)propyl]piperidin-4-yl}acetamide, (253) 2-(4-methoxyphenyl)-N-(4-methylbenzyl)-N-[1-(5-benzofurazanylmethyppiperidin-4-yl]acetamide; (254) 2-(4-methoxyphenyl)-N-(4-methylbenzyl)-N-[1-(5-chlorobenzo[b]thien-3-ylmethyl)piperidin-4-yl]acetamide; (255) 2-(4-methoxyphenyl)-N-(4-methylbenzyl)-N-[1-(5-phenyl-1,2,4-oxadiazol-3-ylmethyl)piperidin-4-yl]acetamide; (256) 2-(4-Chlorophenyl)-N-(4-methylbenzyl)-N-(1-isopropylpiperidin-4-yl)-acetamide; (257) 2-(4-Chlorophenyl)-N-(4-methylbenzyl)-N-(1-ethylpiperidin-4-yl)-acetamide; (258) 2-Phenyl-N-(4-methylbenzyl)-N-(1-methylpiperidin-4-yl)-acetamide; (259) 2-(4-Chlorophenyl)-N-(4-methylbenzyl)-N-(1-methylpiperidin-4-yl)-acetamide; (260) 2-(4-Chlorophenyl)-N-(4-methylbenzyl)-N-(1-cyclopentylpiperidin-4-yl)-acetamide; (261) 2-(4-Fluorophenyl)-N-(4-methylbenzyl)-N-(1-methylpiperidin-4-yl)-acetamide; (262) 2-(4-Chlorophenyl)-N-(4-methylbenzyl)-N-(1-(2-hydroxyethyl)-piperidin-4-yl)-acetamide; (263) 2-(4-Chlorophenyl)-N-(4-methylbenzyl)-N-(1-cyclobutylpiperidin-4-yl)-acetamide; (264) 2-(4-Methoxyphenyl)-N-(4-methylbenzyl)-N-(1-cyclobutylpiperidin-4-yl)-acetamide; (265) 2-(4-Methoxyphenyl)-N-(4-methylbenzyl)-N-(tropin-4-yl)-acetamide; (266) N-(4-Methylbenzyl)-N-(1-methylpiperidin-4-yl)-N′-benzyl-carbamide; (267) N-(4-Methylbenzyl)-N-(1-methylpiperidin-4-yl)-N′-phenyl-carbamide; (268) N-Phenethyl-N-(1-methylpiperidin-4-yl)-N′-benzyl-carbamide; (269) 2-Phenyl-N-(4-methoxybenzyl)-N-(1-methylpiperidin-4-yl)-acetamide; (270) 2-(4-Trifluoromethylphenyl)-N-(4-methoxybenzyl)-N-(1-methylpiperidin-4-yl)-acetamide (271) 2-(4-Fluorophenyl)-N-(4-methoxybenzyl)-N-(1-methylpiperidin-4-yl)-acetamide; (272) 2-(4-Methoxyphenyl)-N-(4-methoxybenzyl)-N-(1-methylpiperidin-4-yl)-acetamide; (273) 2-(4-Methylphenyl)-N-(4-chlorobenzyl)-N-(1-methylpiperidin-4-yl)-acetamide; (274) 2-(4-Hydroxyphenyl)-N-(4-methylbenzyl)-N-(1-methylpiperidin-4-yl)-acetamide; (275) N-Phenethyl-N-(1-methylpiperidin-4-yl)-N′-phenyl-carbamide; (276) N-(3-Phenylpropyl)-N-(1-methylpiperidin-4-yl)-N′-benzyl-carbamide; (277) N-(3-Phenylpropyl)-N-(1-methylpiperidin-4-yl)-N′-phenyl-carbamide; (278) 2-(4-Methoxyphenyl)-2,2-ethylene-N-(4-methylbenzyl)-N-(1-methylpiperidin-4-yl)acetamide; (279) 2-(4-Methoxyphenyl)-N-alpha-methylbenzyl-N-(1-methylpiperidin-4-yl)acetamide; (280) 2-(4-Methoxyphenyl)-N-(4-methylbenzyl)-N-(3-tropen-4-yl)acetamide; (281) 2-Phenyl-2-ethyl-N-(4-methylbenzyl)-N-(1-methylpiperidin-4-yl)acetamide; (282) N-Phenethyl-N-(4-methylbenzyl)-N-(1-methylpiperidin-4-yl)-amine; (283) 2-(4-Methoxyphenyl)-N-(1-indanyl)-N-(1-methylpiperidin-4-yl)acetamide; (284) N-(4-Methylbenzyl)-N-(1-methylpiperidin-4-yl)-N′-(4-methoxybenzyl)-carbamide; (285) 2-(3,4-dimethoxyphenyl)-N-(4-methylbenzyl)-N-(1-methylpiperidin-4-yl)acetamide; (286) 2-(3,4-Methylenedioxyphenyl)-N-(4-methylbenzyl)-N-(1-methylpiperidin-4-yl)acetamide; (287) 2-(4-Methoxyphenyl)-N-(4-methylbenzyl)-N-(1-t-butylpiperidin-4-yl)-acetamide; (288) N-(4-Methylbenzyl)-N-(1-methylpiperidin-4-yl)-N′-phenethyl-carbamide; (289) N-Phenethyl-N-(1-methylpiperidin-4-yl)-N′-phenethyl-carbamide; (290) N-(4-Methylbenzyl)-N-(1-t-butylpiperidin-4-yl)-N′-(4-methoxybenzyl)-carbamide; (291) 2-(4-Ethoxyphenyl)-N-(4-methylbenzyl)-N-(1-methylpiperidin-4-yl)acetamide; (292) 2-(4-Butoxyphenyl)-N-(4-methylbenzyl)-N-(1-methylpiperidin-4-yl)acetamide; (293) 2-(4-i-Propoxyphenyl)-N-(4-methylbenzyl)-N-(1-methylpiperidin-4-yl)acetamide; (294) 2-(4-t-Butoxyphenyl)-N-(4-methylbenzyl)-N-(1-methylpiperidin-4-yl)acetamide; (295) 2-(4-Butoxyphenyl)-N-(4-fluorobenzyl)-N-(1-methylpiperidin-4-yl)acetamide; (296) 2-(4-Propoxyphenyl)-N-(4-fluorobenzyl)-N-(1-methylpiperidin-4-yl)acetamide; (297) 2-(4-i-Propoxyphenyl)-N-(4-fluorobenzyl)-N-(1-methylpiperidin-4-yl)acetamide; (298) 2-(4-t-Butoxyphenyl)-N-(4-fluorobenzyl)-N-(1-methylpiperidin-4-yl)acetamide; (299) 4-(4-Fluorobenzyl)-3-(4-methoxybenzyl)-8-methyl-1-oxa-3,8-diaza-spiro[4.5]decan-2-one; (300) 3-(4-Ethoxybenzyl)-4-(4-fluorobenzyl)-8-methyl-1-oxa-3,8-diaza-spiro[4.5]decan-2-one; (301) 4-(4-Fluorobenzyl)-8-methyl-3-(4-propoxybenzyl)-1-oxa-3,8-diaza-spiro[4.5]decan-2-one; (302) 3-(4-Cyclopropylmethoxybenzyl)-4-(4-fluorobenzyl)-8-methyl-1-oxa-3,8-diaza-spiro[4.5]decan-2-one; (303) 4-(4-Fluorobenzyl)-3-(4-isopropoxybenzyl)-8-methyl-1-oxa-3,8-diaza-spiro[4.5]decan-2-one; (304) 3-(4-Butoxybenzyl)-4-(4-fluorobenzyl)-8-methyl-1-oxa-3,8-diaza-spiro[4.5]decan-2-one; (305) 4-(4-Fluorobenzyl)-3-(4-isobutoxybenzyl)-8-methyl-1-oxa-3,8-diaza-spiro[4.5]decan-2-one; (306) 3-(4-Difluoromethoxybenzyl)-4-(4-fluorobenzyl)-8-methyl-1-oxa-3,8-diaza-spiro[4.5]decan-2-one; (307) 4-(4-Fluorobenzyl)-8-methyl-3-(4-trifluoromethoxybenzyl)-1-oxa-3,8-diaza-spiro[4.5]decan-2-one; (308) 4-(4-Fluorobenzyl)-8-methyl-3-(4-pentoxybenzyl)-1-oxa-3,8-diaza-spiro[4.5]decan-2-one; (309) 8-Ethyl-4-(4-fluorobenzyl)-3-(4-isobutoxybenzyl)-1-oxa-3,8-diaza-spiro[4.5]decan-2-one; (310) 4-(4-Fluorobenzyl)-3-(4-isobutoxybenzyl)-8-isopropyl-1-oxa-3,8-diaza-spiro[4.5]decan-2-one; (311) 8-Cyclopropylmethyl-4-(4-fluorobenzyl)-3-(4-isobutoxybenzyl)-1-oxa-3,8-diaza-spiro[4.5]decan-2-one; (312) 8-Cyclohexylmethyl-4-(4-fluorobenzyl)-3-(4-isobutoxybenzyl)-1-oxa-3,8-diaza-spiro[4.5]decan-2-one; (313) 8-Cyclopentyl-4-(4-fluorobenzyl)-3-(4-isobutoxybenzyl)-1-oxa-3,8-diaza-spiro[4.5]decan-2-one; (314) 4-(4-Fluorobenzyl)-3-(4-isobutoxybenzyl)-8-(3-morpholin-4-yl-propyl)-1-oxa-3,8-diaza-spiro[4.5]decan-2-one; (315) 8-(2-[1,3]Dioxolan-2-yl-ethyl)-4-(4-fluorobenzyl)-3-(4-isobutoxybenzyl)-1-oxa-3,8-diaza-spiro[4.5]decan-2-one; (316) 4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one, (317) 4-(4-Fluorobenzyl)-3-(4-isobutoxybenzyl)-8-[3-(2-oxo-2,3-dihydro-benzoimid azol-1-yl)-propyl]-1-oxa-3,8-diaza-spiro[4.5]decan-2-one; (318) 4-(4-Fluorobenzyl)-3-(4-isobutoxybenzyl)-8-(2-methyl-thiazol-4-yl-methyl)-1-oxa-3,8-diaza-spiro[4.5]decan-2-one; (319) 4-(4-Chlorobenzyl)-3-(4-isobutoxybenzyl)-8-methyl-1-oxa-3,8-diaza-spiro[4.5]decan-2-one; (320) 8-Ethyl-4-(4-chlorobenzyl)-3-(4-isobutoxybenzyl)-1-oxa-3,8-diaza-spiro[4.5]decan-2-one; (321) 4-(4-Chlorobenzyl)-3-(4-isobutoxybenzyl)-8-isopropyl-1-oxa-3,8-diaza-spiro[4.5]decan-2-one; (322) 8-Cyclopropylmethyl-4-(4-chlorobenzyl)-3-(4-isobutoxybenzyl)-1-oxa-3,8-diaza-spiro[4.5]decan-2-one; (323) 8-Cyclohexylmethyl-4-(4-chlorobenzyl)-3-(4-isobutoxybenzyl)-1-oxa-3,8-diaza-spiro[4.5]decan-2-one; (324) 8-(2-[1,3]Dioxolan-2-yl-ethyl)-4-(4-chlorobenzyl)-3-(4-isobutoxybenzyl)-1-oxa-3,8-diaza-spiro[4.5]decan-2-one; (325) 4-(4-Chlorobenzyl)-3-(4-isobutoxybenzyl)-8-[2-(2-oxo-imidazolidin-1-yl)-ethyl]-1-oxa-3,8-diaza-spiro[4.5]decan-2-one; (326) 3-(4-Difluoromethoxybenzyl)-4-(4-fluorobenzyl)-8-methyl-1-oxa-3,8-diaza-spiro[4.5]decan-2-one; (327) 3-(4-Difluoromethoxybenzyl)-8-ethyl-4-(4-fluorobenzyl)-1-oxa-3,8-diaza-spiro[4.5]decan-2-one; (328) 3-(4-Difluoromethoxybenzyl)-4-(4-fluorobenzyl)-8-isopropyl-1-oxa-3,8-diaza-spiro[4.5]decan-2-one; (329) 8-Cyclopropylmethyl-3-(4-difluoromethoxybenzyl)-4-(4-fluorobenzyl)-1-oxa-3,8-diaza-spiro[4.5]decan-2-one; (330) 8-Cyclohexylmethyl-3-(4-difluoromethoxybenzyl)-4-(4-fluorobenzyl)-1-oxa-3,8-diaza-spiro[4.5]decan-2-one; (331) 3-(4-Difluoromethoxybenzyl)-8-(2-[1,3]dioxolan-2-yl-ethyl)-4-(4-fluorobenzyl)-1-oxa-3,8-diaza-spiro[4.5]decan-2-one; (332) 3-(4-Difluoromethoxybenzyl)-4-(4-fluorobenzyl)-8-[2-(2-oxo-imidazolidin-1-yl)-ethyl]-1-oxa-3,8-diaza-spiro[4.5]decan-2-one; (333) 8-Ethyl-4-(4-fluorobenzyl)-3-(4-trifluoromethoxybenzyl)-1-oxa-3,8-diaza-spiro[4.5]decan-2-one; (334) 4-(4-Fluorobenzyl)-8-isopropyl-3-(4-trifluoromethoxybenzyl)-1-oxa-3,8-diaza-spiro[4.5]decan-2-one; (335) 8-Cyclopropylmethyl-4-(4-fluorobenzyl)-3-(4-trifluoromethoxybenzyl)-1-oxa-3,8-diaza-spiro[4.5]decan-2-one; (336) 8-Cyclohexylmethyl-4-(4-fluorobenzyl)-3-(4-trifluoromethoxybenzyl)-1-oxa-3,8-diaza-spiro[4.5]decan-2-one; (337) 8-Cyclopentyl-4-(4-fluorobenzyl)-3-(4-trifluoromethoxybenzyl)-1-oxa-3,8-diaza-spiro[4.5]decan-2-one; (338) 8-(2-[1,3]Dioxolan-2-yl-ethyl)-4-(4-fluorobenzyl)-3-(4-trifluoromethoxybenzyl)-1-oxa-3,8-diaza-spiro[4.5]decan-2-one; (339) 4-(4-Fluorobenzyl)-8-[2-(2-oxo-imidazolidin-1-yl)-ethyl]-3-(4-trifluoromethoxybenzyl)-1-oxa-3,8-diaza-spiro[4.5]decan-2-one; (340) 8-Ethyl-4-(4-fluorobenzyl)-3-(4-propoxybenzyl)-1-oxa-3,8-diaza-spiro[4.5]decan-2-one; (341) 4-(4-Fluorobenzyl)-8-isopropyl-3-(4-propoxybenzyl)-1-oxa-3,8-diaza-spiro[4.5]decan-2-one; (342) 8-Cyclopropylmethyl-4-(4-fluorobenzyl)-3-(4-propoxybenzyl)-1-oxa-3,8-diaza-spiro[4.5]decan-2-one; (343) 8-Cyclohexylmethyl-4-(4-fluorobenzyl)-3-(4-propoxybenzyl)-1-oxa-3,8-diaza-spiro[4.5]decan-2-one; (344) 8-Cyclopentyl-4-(4-fluorobenzyl)-3-(4-propoxybenzyl)-1-oxa-3,8-diaza-spiro[4.5]decan-2-one; (345) 8-(2-[1,3]Dioxolan-2-yl-ethyl)-4-(4-fluorobenzyl)-3-(4-propoxybenzyl)-1-oxa-3,8-diaza-spiro[4.5]decan-2-one; (346) 4-(4-Fluorobenzyl)-8-[2-(2-oxo-imidazolidin-1-yl)-ethyl]-3-(4-propoxybenzyl)-1-oxa-3,8-diaza-spiro[4.5]decan-2-one; (347) 3-(4-Cyclopropylmethoxybenzyl)-8-ethyl-4-(4-fluorobenzyl)-1-oxa-3,8-diaza-spiro[4.5]decan-2-one; (348) 3-(4-Cyclopropylmethoxybenzyl)-4-(4-fluorobenzyl)-8-isopropyl-1-oxa-3,8-diaza-spiro[4.5]decan-2-one; (349) 3-(4-Cyclopropylmethoxybenzyl)-8-cyclopropylmethyl-4-(4-fluorobenzyl)-1-oxa-3,8-diaza-spiro[4.5]decan-2-one; (350) 3-(4-Cyclopropylmethoxybenzyl)-8-(2-[1,3]dioxolan-2-yl-ethyl)-4-(4-fluorobenzyl)-1-oxa-3,8-diaza-spiro[4.5]decan-2-one; (351) 3-(4-Cyclopropylmethoxybenzyl)-4-(4-fluorobenzyl)-8-[2-(2-oxo-imidazolidin-1-yl)-ethyl]-1-oxa-3,8-diaza-spiro[4.5]decan-2-one; (352) 8-(2-[1.3]-Dioxan-2-yl-ethyl)-4-(4-fluorobenzyl)-3-(4-isobutoxybenzyl)-1-oxa-3,8-diaza-spiro[4.5]decane-3-one; (353) 4-(4-Fluorobenzyl)-3-(4-isobutoxybenzyl)-8-{3-[(S)-4-isopropyl-2-oxo-oxazolidin-3-yl]-propyl}spiro[4.5]decane-3-one; (354) N-{1-[2-(1,3-Dioxolan-2-yl)ethyl]piperidin-4-yl}-N-(4-fluorobenzyl)-N′-(4-isobutoxybenzyl)carbamide hydrochloride; (355) N-{1-[2-(1,3-Dioxan-2-yl)ethyl]-piperidin-4-yl}-N-(4-fluorobenzyl)-2-[4-(2-hydroxy-2-methylpropoxy)phenyl]-acetamide tartrate; (356) N-(4-Fluorobenyzl)-N-(piperidin-4-yl)-2-(4-isobutoxyphenyl)acetamide; (357) N-{1-[3-(3,5-Dimethylpiperidin-1-yl)propyl]piperidin-4-yl-}-N-(4-fluorobenzyl)-2-(4-isobutoxyphenyl)acetamide dihydrochloride; (358) 1-[3-(4-{(4-Fluorobenzyl)-[2-(4 isobutoxyphenyl)acetyl]amino}piperidin-1-yl)propyl]piperidine-4-carboxylic acid methyl ester dihydrochloride; (359) N-(4-Fluorobenzyl)-2-(4-isobutoxyphenyl)-N-{1-[2-(1-methylpyrrolidin-2-yl-)ethyl]piperidin-4-yl}acetamide dioxalate; (360) N-{1-[3-(2,6-Dimethylmorpholin-4-yl)propyl]piperidin-4-yl}-N-(4-fluorobenzyl)-2-(44 sobutoxyphenyl)acetamide dioxalate; (361) N-(4-Fluorobenzyl)-N-{1-[3-(3-hydroxypiperidin-1-yl)propyl]piperidin-4-yl}-2-(4-isobutoxyphenyl)acetamide dioxalate; (362) N-(4-Fluorobenzyl)-2-(4-isobutoxyphenyl)-N-{1-[3-(2-methylpiperidin-1-yl)-propyl]piperidin-4-yl}acetamide dioxalate; (363) N-(4-Fluorobenzyl-2-(4-isobutoxyphenyl)-N-[1-(3-pyrrolidin-1-yl-propyl)piperidin-4-yl]acetamide dioxalate; (364) N-{1-[3-(2,5-Dimethylpyrroli din-1-yl)propyl]piperidin-4-yl}-N-(4-fluorobenzyl)-2-(4-isobutoxyphenyl)acetamide dioxalate; (365) N-(4-Fluorobenzyl)-N-{1-[3-(3-hydroxymethylpiperidin-1-yl)propyl]piperidin-4-yl}-2-(4-isobutoxyphenyl)acetamide dioxalate; (366) N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide oxalate; (367) N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide oxalate; (368) N-[2-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide oxalate; (369) N-(4-Fluorobenzyl)-N-{1-[3-(4-(S)-isopropyl-2-oxo-oxazolidin-3-yl)propyl]piperidin-4-yl}-2-(4-propoxyphenyl)acetamide oxalate; (370) N-{1-[2-(1,3-Dioxan-2-yl)ethyl]piperidin-4-yl}-N-(4-fluorobenzyl)-2-(4-isobutoxyphenyl)acetamide oxalate; (371) N-{1-[2-(1,3-Dioxan-2-yl)ethyl]piperidin-4-yl}-N-[2-(4-fluorophenyl)ethyl]-2-(4-isobutoxyphenyl)acetamide oxalate; (372) N-{1-[2-(1,3-Dioxan-2-yl)ethyl]piperidin-4-yl}-N-[2-(4-fluorophenyl)ethyl]-2-(4-propoxyphenyl)acetamide oxalate; (373) N-{1-[2-(1,3-Dioxan-2-yl-)ethyl]piperidin-4-yl}-N-(4-fluorobenzyl)-2-(4-propoxyphenyl)acetami de tartrate; (374) N-{1-[2-(1,3-Dioxan-2-yl)ethyl]piperidin-4-yl}-N-(4-fluorobenzyl)-N′-(4-isobutoxybenzyl)carbami de tartrate; (375) N-{1-[2-(1,3-Dioxan-2-yl)ethyl]piperidin-4-yl}-N-(4-fluorobenzyl)-2-(4-fluorophenyl)acetamide tartrate; (376) N-{1-[2-(1,3-Dioxan-2-yl)ethyl]piperidin-4-yl}-N-(4-fluorobenzyl)-2-p-tolylacetamide tartrate; (377) 2-Benzofuran-5-yl-N-{1-[2-(1,3-dioxan-2-yl)ethyl]piperidin-4-yl}-N-(4-fluorobenzyl)acetamide tartrate; (378) 2-(2,3-Dihydrobenzofuran-5-yl)-N-{1-[2-(1,3-dioxan-2-yDethyl]piperidin-4-yl}-N-(4-fluorobenzyl)acetamide tartrate; (379) N-{1-[2-(2,2-Dimethyl-1,3-dioxolan-4-yl)ethyl]piperidin-4-yl}-N-(4-fluorobenzyl)-2-(4-isobutoxyphenyl)acetamide tartrate; (380) N-{1-[2-(1,3-Dioxan-4-yl)ethyl]piperidin-4-yl}-N-(4-fluorobenzyl)amine; (381) N-{1-[2-(1,3-Dioxan-4-yl)ethyl]piperidin-4-yl}-N-(4-fluorobenzyl)-2-(4-isobutoxyphenyl)acetamide tartrate; (382) N-{1-[2-(1,3-Dioxan-4-yl)ethyl]piperidin-4-yl}-N(4-fluorobenzyl)-2-(4-trifluoromethylphenyl)acetamide tartrate; (383) 2-(4-Cyanophenyl)-N-{1-[2-(1,3-dioxan-4-yl)ethyl]piperidin-4-yl}-N-(4-fluorobenzyl)acetamide tartrate; (384) N-(4-Fluorobenzyl)-2-(4-isobutoxyphenyl)-N-{1-[2-(2-oxo-imidazolidin-1-yl)ethyl]piperidin-4-yl}acetamide hydrochloride; (385) 2-(4-Methoxyphenyl)-N-(4-methylbenzyl)-N-{1-[2-(2-oxo-imidazolidin-1-yl)ethyl]piperidin-4-yl}acetamide hydrochloride; (386) N-(4-Fluorobenzyl)-2-(4-isopropoxyphenyl)-N-{1-[2-(2-oxo-imidazolidin-1-yl)ethyl]piperidin-4-yl}acetamide hydrochloride; (387) N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide hydrochloride; (388) N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide hydrochloride; (389) 2-(4-Methoxyphenyl)-N-(4-methylbenzyl)-N-{1-[3-(2-oxo-2,3-dihydrobenzoimidazol-1-yl)propyl]piperidin-4-yl}-acetamide hydrochloride; (390) N-(4-Fluorobenzyl)-2-(4-isopropoxyphenyl)-N-{1-[4-(2-oxo-2,3-dihydrobenzoimidazol-1-yl)butyl]piperidin-4-yl}acetamide hydrochloride; (391) N-{1-[2-(2,4-Dioxo-1,4-dihydro-2H-quinazolin-3-yl)ethyl]piperidin-4-yl}-N-(4-fluorobenzyl)-2-(4-isopropoxyphenyl)acetamide hydrochloride; (392) 4-(4-Fluorobenzylamino)-piperidine-1-carboxylic acid benzyl ester; (393) N-(1-Benzyloxycarbonylpiperidin-4-yl)-N-(4-fluorobenzyl)-N′-(4-isopropoxybenzyl)carbamide; (394) N-(4-Fluorobenzyl)-N′-(4-isopropoxybenzyl)-N-piperidin-4-yl-carbamide oxalate; (395) N-{1-[2-(1,3-Dioxolan-2-yl)ethyl}piperidin-4-yl]-N-(4-fluorobenzyl)-N′-(4-isopropoxy-benzyl)carbamide oxalate; methoxyphenyl)-N-(4-methylbenzyl)acetamide hydrochloride; (397) N-{1-[2-(1,3-Dioxolan-2-yl-)ethyl]piperidin-4-yl}-N-(4-fluorobenzyl)-2-(4-isobutoxyphenyl)acetamide hydrochloride; (398) N-{1-[2(1,3-Dioxolan-2-yl)ethyl]piperidin-4-yl}-2-(4-isopropoxyphenyl)-N-(4-methylbenzyl)acetamide hydrochloride; (399) N-{1-[2-(1,3-Dioxolan-2-yeethyl]piperidin-4-yl}-N-(4-fluorobenzyl)-2-(4-propoxyphenyl)acetamide tartrate; (400) N-(4-Fluorobenzyl)-N′-(4-isopropoxybenzyl)-N-{1-[2-((S)-4-methyl-1,3-dioxolane-2-yl)ethyl]piperidin-4-yl}-carbamide oxalate; (401) N-(4-Fluorobenzyl)-N′-(4-isopropoxybenzyl)-N-[1-(3-morpholin-4-yl-propyl)piperidin-4-yl]carbamide oxalate; (402) 2-(4-Methoxyphenye-N-(4-methylbenzyl)-N-[1-(2-morpholin-4-yl-ethyppiperidin-4-yl]acetamide dihydrochloride; (403) 2-(4-Methoxyphenyl)-N-(4-methylbenzyl)-N-[1-(3-morpholin-4-ylpropyl)piperidin-4-yl]acetamide dihydrochloride; (404) N-(4-Fluorobenzyl)-2-(4-isobutoxyphenyl)-N-[1-(3-morpholin-4-ylpropyl)piperidin-4-yl]acetamide dihydrochloride; (405) N-(4-Fluorobenzyl)-2-(4-isopropoxy-phenyl)-N-[1-(3-morpholin-4-yl-propyl)piperidin-4-yl]acetamide dihydrochloride; (406) N-(4-Fluorobenzyl)-N′-(4-isopropoxybenzyl)-N-[1-(3-piperidin-1-yl-propyl)piperidin-4-yl]carbamide oxalate; (407) N-(4-Fluorobenzyl)-N′-(4-isopropoxybenzyl)-N-[1-(3-((S)-4-isopropyl-2-oxazolidinon-1-yl-propyl)piperidin-4-yl]carbamide tartrate; (408) N-(4-Fluorobenzyl)-N′-(4-isopropoxybenzyl)-N-{1-[2-(2,5,5-trimethyl-1,3-dioxan-2-yl)ethyl]lpiperidin-4-yl}carbamide oxalate; (409) N-{1-[3-(1,3-Dioxolan-2-yl)propyl]piperidin-4-yl}-N-(4-fluorobenzyl)-N′-(4-isopropoxybenzyl)carbamide oxalate; (410) N-[1-(2,2-Dimethyl-1,3-dioxan-5-yl)-piperidin-4-yl]-N-(4-fluorobenzyl)-N′-(4-isopropoxybenzyl)carbamide oxalate; (411) N-(4-Fluorobenzyl)-N′-(4-isopropoxybenzyl)-N-{[2-(1-methylpyrrolidin-2-yl)ethyl]-piperidin-4-yl}carbamide oxalate; (412) N-[1-(2,2-Dimethyl-1,3-dioxan-5-yl)piperidin-4-yl]-N-(4-fluorobenzyl)-2-(4-isobutoxyphenyl)acetamide oxalate; (413) N-[1-(1,3-Dioxan-5-yl)-piperidin-4-yl)-N-(4-fluorobenzyl)-2-(4-isobutoxyphenyl)acetamide tartrate; (414) N-[1-(2,2-Dimethyl-1,3-dioxan-5-yl)piperidin-4-yl]-N-(4-fluorobenzyl)-2-(4-fluorophenyl)acetamide tartrate; (415) N-{1-[2-(1,3-Dioxan-4-yl)ethyl]piperidin-4-yl}-N-(4-fluorobenzyl)-2-(4-fluorophenyl)acetamide tartrate: (416) N-{1-[2-(1,3-Dioxan-4-yl)ethyl]piperidin-4-yl}-N-(4-fluorobenzyl)-2-(4-trifluoromethoxyphenyl)acetamide tartrate: (417) N-{1-[2-(1,3-Dioxan-4-yl)ethyl]piperidin-4-yl}-N-(4-fluorobenzyl)-2-(4-propoxyphenyl)acetamide tartrate; (418) N-(4-Fluorobenzyl)-2-(4-isobutoxyphenyl)-N-[1-(tetrahydropyran-4-yppiperidin-4-yl]acetamide tartrate; (419) N-(4-Fluorobenzyl)-2-(4-isobutoxyphenyl)-N-[1-(tetrahydropyran-4-ylmethyl)piperidin-4-yl]acetamide tartrate; (420) N-(4-Fluorobenzyl)-2-(4-isobutoxyphenyl)-N-{1-[2-(tetrahydropyran-4-yl)ethyl]piperidin-4-yl]acetamide tartrate; (421) N-(4-Fluorobenzyl)-2-(4-fluorophenyl)-N-[1-(tetrahydropyran-4-yl)piperidin-4-yl]acetamide tartrate; (422) N-[14(S)-3,5-Dihydroxypentyppiperidine-4-yl]-N-(4-fluorobenzyl)-2-(4-isobutoxyphenyl)acetamide tartrate; (423) N-{1-[2-((4S)-1,3-Dioxane-4-yl)ethyl]piperidine-4-yl}-N-(4-fluorobenzyl)-2(4-isobutoxyphenyl)acetamide tartrate; (424) N-{1-[2-(1,3-Dioxan-2-yl)ethyl]piperidin-4-yl}-N-(4-fluorobenzyl)amine; (425) 2-(4-Benzyloxyphenyl)-N-{1-[2-(1,3-dioxan-2-yl)ethyl]piperidin-4-yl}-N-(4-fluorobenzyl)acetamide tartrate; (426) N-{1-[2-(1,3-Dioxan-2-yl)ethyl]piperidin-4-yl}-N-(4-fluorobenzyl)-2-(4-hydroxyphenyl)-acetamide tartrate; (427) N-{1-[2-(1,3-Dioxan-2-yl)ethyl]piperidin-4-yl}-N-(4-fluorobenzyl)-2-(4-methoxyphenyl)-acetamide tartrate; (428) N-{1-[2-(1,3-Dioxan-2-yl)ethyl]piperidin-4-yl}-N-(4-fluorobenzyl)-2-(4-isopropylphenyl)-acetamide tartrate; (429) N-{1-[2-(1,3-Dioxan-2-yl)ethyl]piperidin-4-yl}-N-(4-fluorobenzyl)-2-(4-trifluoromethoxy-phenyl)acetamide tartrate; (430) N-{1-[2-(1,3-Dioxan-2-yl)ethyl]-piperidin-4-yl}-N-(4-fluorobenzyl)-2-(4-ethoxyphenyl)-acetamide oxalate; (431) N-{1-[2-(1,3-Dioxan-2-yl)ethyl]piperidin-4-yl}-N-(4-fluorobenzyl)-2-(4-isopropoxyphenyl)-acetamide oxalate; (432) N-{1-[2-(1,3-Dioxan-2-yl)ethyl]piperidin-4-yl}-N-(4-fluorobenzyl)-2-phenylacetamide oxalate; (433) N-{1-[2-(1,3-Dioxan-2-yl)ethyl]piperidin-4-yl}-N-(4-fluorobenzyl)-2-[4-(2-fluoroethoxy)-phenyl]acetamide oxalate; (434) N-{1-[2-(5,5-Dimethyl-1,3-dioxan-2-yl)ethyl]piperidin-4-yl}-N-(4-fluorobenzyl)-2-(4-isobutoxyphenyl)acetamide oxalate; (435) N-(4-Fluorobenzyl)-2-(4-isobutoxyphenyl)-N-{1-[2-((R)-4-methyl-1,3-dioxan-2-yl)ethyl]-piperidin-4-yl}acetamide oxalate; (436) N-(4-Fluorobenzyl)-2-(4-isobutoxyphenyl)-N-{1-[2-((S)-4-methyl-1,3-dioxolan-2-yl)ethyl]piperidin-4-yl}acetamide oxalate; (437) N-{1-[2-(4,6-Dimethyl-1,3-dioxan-2-yl)ethyl]piperidin-4-yl}-N-(4-fluorobenzyl)-2-(4-isobutoxyphenyl)acetamide oxalate; (438) N-(4-Fluorobenzyl)-N-{1-[2-((S)-4-methyl-1,3-dioxolan-2-yl)ethyl]piperidin-4-yl}-2-(4-trifluoromethoxyphenyl)acetamide oxalate; (439) N-(4-Fluorobenzyl)-2-(4-isopropylphenyl)-N-{1-[2-((S)-4-methyl-1,3-dioxolan-2-yl)ethyl]-piperidin-4-yl}acetamide oxalate; (440) N-(4-Fluorobenzyl)-N-{1-[2-((R)-4-methyl-1,3-dioxan-2-yl)ethyl]piperidin-4-yl}-2-(4-trifluoromethoxyphenyl)acetamide oxalate; (441) N-(4-Fluorobenzyl)-2-(4-isobutoxyphenyl)-N-{1-[2-(2,5,5-trimethyl-1,3-dioxan-2-yl)ethyl]piperidin-4-yl}acetamide oxalate; (442) N-(4-Fluorobenzyl)-2-(4-isobutoxyphenyl)-N-{1-[2-(2-methyl-1,3-dioxolan-2-yl)ethyl]-piperidin-4-yl-}acetamide oxalate; (443) N-(4-Fluorobenzyl)-2-(4-isobutoxyphenyl)-N-{1-[3-(1,3-dioxolan-2-yl)propyl]piperidin-4-yl}acetamide tartrate; (444) N-(4-Fluorobenzyl)-2-(4-isobutoxyphenyl)-N-{1-(3-piperidin-1-yl-propyl)piperidin-4-yl}-acetamide dihydrochloride; (445) N-(4-Fluorobenzyl)-2-(4-isobutoxyphenyl)-N-{1-[2-(tetrahydropyran-2-yloxy)ethyl]-piperidin-4-yl}acetamide oxalate; (446) N-(4-Fluorobenzyl)-2-(4-isobutoxyphenyl)-N-{1-[3-(2-oxo-piperidin-1-yepropyl]piperidin-4-yl}acetamide; (447) N-(4-Fluorobenzyl)-2-(4-isobutoxyphenyl)-N-{1-[3-(2-oxo-pyrrolidin-1-yl)propyl]piperidin-4-yl}acetamide hydrochloride; (448) N-(4-Fluorobenzyl)-2-(4-isobutoxyphenyl)-N-{1-[3-((R)-4-isopropyl-2-oxo-oxazolidin-3-yepropyl]piperidin-4-yl}acetamide oxalate; (449) N-(4-Fluorobenzyl)-2-(4-isobutoxyphenyl)-N-{1-[3-(2-oxo-oxazolidin-3-yl)propyl]piperidin-4-yl}acetamide oxalate; (450) N-(4-Fluorobenzyl)-2-(4-isobutoxyphenyl)-N-{1-[3-((S)-4-methyl 2-oxo-oxazolidin-3-yl)propyl]piperidin-4-yl}acetamide tartrate; (451) N-(4-Fluorobenzyl)-2-(4 isobutoxyphenyl)-N-{1-[3-((S)-4-ethyl-2-oxo-oxazolidin-3-yl)-propyl]piperidin-4-yl}acetamide oxalate; (452) N-(4-Fluorobenzyl)-2-(4-isobutoxyphenyl)-N-{1-[2-(1,3-oxothiolan-2-yl)ethyl]piperidin-4-yl}acetamide L-tartrate; (453) 2-(4-Bromophenyl)-N-{1-[2-(1,3-dioxan-2-yl)ethyl)piperidin-4-yl}-N-(4-fluorobenzyl)-acetamide L-tartrate; (454) N-{1-[2-(1,3-Dioxan-2-yl)ethyl)piperidin-4-yl}-N-(4-fluorobenzyl)-2-(4-isobutylamino-phenyl)acetamide L-tartrate; (455) N-{1-[2-(1,3-Dioxan-2-yl)ethyl)piperidin-4-yl}-N-(4-fluorobenzyl)-2-(4-propylaminophenyl)acetamide L-tartrate; (456) N-{1-[2-(1,3-Dioxan-2-yl)ethyl)piperidin-4-yl}-N-(4-fluorobenzyl)-2-(4-(1-nitropropyl)-phenyl)acetamide L-tartrate; (457) N-{1-[2 (1,3-Dioxan-2-yl)ethyl)piperidin-4-yl}-N-(4-fluorobenzyl)-2-[4-(2-oxopyrrolidin-1-yl)phenyl)acetamide L-tartrate; (458) N-{1-[2-(1,3-Dioxan-2-yl)ethyl)piperidin-4-yl}-N-(4-fluorobenzyl)-2-(4-isobutylsulfanylphenyl)acetamide L-tartrate; (459) N-{1-[2-(1,3-Dioxan-2-yl)ethyl)piperidin-4-yl}-N-(4-fluorobenzyl)-2-(4-iodophenyl)-acetamide L-tartrate; (460) 2-(4-Acetophenyl)-N-{1-[2-(1,3-dioxan-2-yl)ethyl)piperidin-4-yl}-N-(4-fluorobenzyl)-acetamide L-tartrate; (461) 2-[4-(1-hydroxyiminoethyl)phenyl]-N-{1-[2-(1,3-dioxan-2-yl)ethyl)piperidin-4-yl}-N-(4-fluorobenzyl)acetamide L-tartrate; (462) N-{1-[2-(1,3-Dioxan-2-yl)ethyl)piperidin-4-yl}-N-(4-fluorobenzyl)-2-(4-morpholin-4-yl-phenyl)acetamide L-tartrate; (463) N-{1-[2-(1,3-Dioxan-2-yl)ethyl)-piperidin-4-yl}-N-(4-fluorobenzyl)-2-(4-pyrazol-1-ylphenyl)acetamide L-tartrate; (464) N-{1-[2-(1,3-Dioxan-2-yl)-1-methylethyl]piperidin-4-yl}-N-(4-fluorobenzyl)-2-(4-isobutoxyphenyl)-acetamide L-tartrate; (465) N-{1-[2-(1,3-Dioxan-4-yl)ethyl)piperidin-4-yl}-N-(4-fluorobenzyl)-2-(4-pyrazol-1-ylphenyl)acetamide L-tartrate; (466) N-[1-((R)-3,5-Dihydroxypentyl)piperidine-4-yl]-N-(4-fluorobenzyl)-2-(4-isobutoxyphenyl)acetamide tartrate; (467) N-{1-[2-((4R)-1,3-Dioxane-4-yl)ethyl]piperidine-4-yl}-N-(4-fluorobenzyl)-2-(4-isobutoxyphenyeacetamide tartrate; (468) N-{1-[2-(1,3-Dioxan-2-yl)ethyl]piperidin-4-yl}-N-(4-fluorobenzyl)-2-[4-(1,2,4-triazol-4-yl)phenyl]acetamide L-tartrate; (469) nortriptyline; (470) duloxetine; (471) lofepramine; (472) tomoxetine; (473) 3-({1-[2-(7-methyl-5-oxo-5H)-[1,3]thiazolo[3,2-a]pyrimidin-6-yl)ethyl]-3-pyrrolidinyl}methyl)-1H-indole-5-carbonitrile hydrochloride; (474) 3-({1-[2-(6-chloro-2-oxo-2,3-dihydro-1H-indol-5-yl)ethyl]-3-pyrrolidinyl}-methyl)-1H-indole-5-carbonitrile hydrochloride; (475) moclobemide; (476) N-acetylserotonin; (477) bromfaromine; (478) beflaxozone; (479) chlorimipramine; (480) cyanimipramine; (481) cianopramine; (482) desipramine; (483) protriptyline; (484) trimipramine; (485) doxepin; (486) cyclobenzaprine; (487) 5-methoxycarbonylamino-N-acetyltryptamine; (488) amoxapine; (489) maprotiline; (490) fefazodone; (491) flesinoxan hydrochloride; (492) urapidil; (493) WY47846 (3a,4,4a,6a,7,7a-hexahydro-2-[4-[4-(2-pyrimidinyl)-1-piperazinyl]-butyl]-4,7-etheno-1H-cyclobutano[f]isoindole-1,3(2H)-dione dihydrochloride sesquihydrate); (494) SM3997 (N-[4-[4-(2-pyrimidinyl)-1-piperazinyl]butyl]-bicyclo[2.2.1]heptane-2,3-di-exo-carboximide); (495) 2-(4-(4-(2-pyrimidinyl)-1-piperazinyl-propyl)-1,2-benzoisothiazol-3-(2H)-one 1,1-dioxide hydrochloride; (496) KC9172 (3-butyl-9,9-dimethyl-7-[4-[4-[2-methoxyphenyl)-1-piperazinyl]butyl]-3,7-diazabicyclo[3,2,1]nonane-2,4,6,8-tetraone); (497) 4-(N,N-dipropylamino)-6-methoxy-1,3,4,5-tetrahydrobenz-[c,d]indole; (498) 4-[4-(N-1,2-benzisothiazol-3(2H)-one 1,1-dioxido)]butylamino-6-methoxy-1,3,4,5-tetrahydrobenz[c,d]-indole hydrochloride; (499) 5-carboxamidotryptamine; (500) N,N-dipropyl-5-carboxamidotryptamine; (501) AH25086 (3-(2-aminoethyl)-1H-indole-5-(N-methyl)acetamide); (502) GR43175 (3-(2-dimethylaminoethyl)-1H-indole-5-(N-methyl)methanesulfonamide); (503) 3-(2-[4-[2-(1,2-benzisothiazole-3(2H)-one 1,1-dioxido)]butyl]amino)ethyl-5-methoxy-1H-indole; (504) spiroxatrine; (505) MDL72832 (8-[4-(1,4-benzodioxan-2-ylmethylamino)butyl]-8-azaspiro-[4,5]decane-7,9-dione); (506) 2-[4-(1,4-benzodioxan-2-ylmethylamino)butyl]-1,2-benzisothiazol-3(2H)-one 1,1-dioxide; (507) 2-(N,N-dipropylamino)-8-hydroxy-1,2,3,4-tetrahydronaphthalene; (508) 2-{4-[2-(1,2-benzisothiazol-3(2H)-one 1,1-dioxido)]butyl}amino-8-methoxy-1,2,3,4-tetrahydronaphthalene; (509) 3-N,N-dipropylamino-5-hydroxy-thiochroman; 3-N,N-dipropylamino-5-ethoxy-thiochroman; (510) 3-N,N-dipropylamino-5-ethoxychroman; (511) 1-[2-(3-indolyl)]-ethyl-2,6-dimethyl-piperidine; (512) 1-{2-[3-(5-carboxamido)indolyl]}ethyl-2,6-dimethylpiperidine; (513) RU24924 (5-methoxy-3-(1,2,3,6-tetrahydropyridin-4-yl]-1H-indole); (514) 5-methoxy-3-(1,2,3,6-tetrahydropyridin-5-yl)-1H-indole; (515) diethyl N-benzyloxycarbonyl-5-benzyloxycarbonyloxy-L-tryptophyl-L-aspartate; (516) dibenzyl N-benzyloxycarbonyl-5-hydroxy-L-tryptophanylaspartate; (517) 5-Hydroxy-L-tryptophyl-L-aspartic acid trihydrate; (518) diethyl N-benzyloxycarbonyl-5-hydroxy-L-tryptophyl-L-glutamate; (519) diethyl 5-hydroxy-L-tryptophyl-L-glutamate hydrochloride; (520) dibenzyl L-benzyloxycarbonyl-5-hydroxytryptophyl-L-glutamate; (521) 5-hydroxy-L-tryptophyl-L-glutamic acid; (522) pentachlorophenyl ester of N-benzyloxycarbonyl-5-hydroxy-L-tryptophan; (523) methyl ester of N-benzyloxycarbonyl-5-hydroxy-L-tryptophyl-L-tyrosine; (524) N-Acetyl-5-hydroxy-L-tryptophan; (525) methyl ester of N-acetyl-5-hydroxy-L-tryptophyl-L-tyrosine; (526) methyl ester of N-acetyl-5-hydroxy-L-tryptophyl-5-hydroxy-L-tryptophan; (527) 5-hydroxy-L-tryptophyl-L-alanine hydrate; (528) 5-hydroxy-L-tryptophan-L-valine; (529) 5-hydroxy-L-tryptophyl-L-leucine; (530) 5-hydroxy-L-tryptophyl-L-proline; (531) 5-hydroxy-L-tryptophyl-L-phenylalanine; (532) 5-hydroxy-L-tryptophyl-5-hydroxy-L-tryptophan; (533) 5-hydroxy-L-tryptophyl-L-tryptophan; (534) 1-(5-hydroxy)tryptophyl-L-serine; (535) 5-hydroxy-L-tryptophyl-L-arginine; (536) 5-hydroxy-L-tryptophylglycine; (537) 5-hydroxy-1-tryptophyl-gamma-aminobutyric acid; (538) 5-hydroxy-L-tryptophanamide hydrate; (539) methyl ester of 5-hydroxy-L-tryptophyl-L-histidine; (540) benzyl ester of L-5-hydroxytryptophan; (541) benzyl ester of N-benzyloxycarbonyl-5-hydroxy-L-tryptophyl-5-hydroxy-L-tryptophan; (542) 5-Hydroxy-L-tryptophyl-5-hydroxy-L-tryptophan hemihydrate; (543) 5-hydroxytryptophan inosinate; (544) theophylline salt of (DL) 5-hydroxytryptophan; (545) RU25591 (6,7,8,9-tetrahydro N,N-dimethyl 5-[4-nitrophenyl]oxy 5H-benzocyclohepten 7-amine) cis-fumarate); (546) LM5008 (4-[2-(3-indolyl)ethyl]piperidine); (547) DU24565 (6-nitro-2-(1-piperazinyl)quinoline); (548) CGP6085/A (4-(5,6-dimethyl-2-benzofuranyl)piperidine hydrochloride); (549) alaprociate; (550) dibenzoxazepine; (551) deprenyl; (552) isocarboxazide; (553) furazolidone; (554) procarbazine; (555) Ro 60-0175/ORG 35030 ((S)-2-(4,4,7-trimethyl-1,4-dihydro-indeno (1,2-B) pyrrol-1-yl)-1-methyl-ethylamine) (556) Ro 60-0332/ORG 35035 ((S)-2-(Chloro-5-fluoro-indol-1-yl)-1-methylethylamine); (557) 1-[6-Chloro-5-trifluoromethyl)-2-pyridinyl]-piperazine hydrochloride; (558) 5-carboxyamidotryptamine; (559) SB 206553 (3,5-Dihydro-5-methyl-N-3-pyridinylbenzo[1,2-b:4,5-H]dipyrrole-1(2H)-carboxamide hydrochloride); (560) ondansetron; (561) granisetron; (562) tropisetron; (563) dolasetron; (564) palonosetron; (565) trimethobenzamide; (566) risperidone; (567) clozapine; (568) azatadine; (569) cyproheptadine; (570) fenclonine; (571) chlorpromazine; (572) (3β)-2,3-dihydrolysergine; (573) (30)-2,3-dihydroisolysergine, (574) (3β, 5β, 8β)-9,10-didehydro-2,3-dihydro-6-methylergoline-8-acetonitrile; (575) 251-NBMD (2-(4-iodo-2,5-dimethoxyphenyl)-N-[(2,3-methylenedioxyphenyl)methyl]ethanamine); (576) N-(2-methoxybenzyl)-1-(8-bromo-2,3,6,7-tetrahydrobenzo[1,2-b:4,5-b′]difuran-4-yl)-2-aminoethane; (577) 5-benzyloxytryptamine; (578) 5-methoxy-7-N,N-dimethyltryptamine; (579) A372159 ((11S,16R)-3-[4-(propan-2-yloxy)-2-(trifluoromethyl)phenyl]-6-oxa-10,14-diazatetracyclo[8.6.1.0^{5,17}.0^{11,16}]heptadeca-1,3,5(17)-triene); (580) AL-34662 (14(S)-2-Aminopropyl)-1H-indazol-6-ol); (581) AL-37350A ((S)-(+)-1-(2-Aminopropyl)-8,9-dihydropyrano[3,2-e]indole); (582) AL-38022A ((S)-2-(8,9-dihydro-7H-pyrano[2,3-g]ind a zol-1-yl)-1-methylethylamine); (583) AS-19 ((2S)—N,N-dimethyl-5-(1,3,5-trimethylpyrazol-4-yl)-1,2,3,4-tetrahydronaphthalen-2-amine); (584) alnespirone; (585) BIMU8 (N-[(1R,5S)-8-methyl-8-azabicyclo[3.2.1]oct-3-yl]-2-oxo-3-(propan-2-yl)-2,3-dihydro-1H-benzimidazole-1-carboxamide hydrochloride); (586) BMY-14802 (1-(4-fluorophenyl)-4-[4-(5-fluoropyrimidin-2-yl)piperazin-1-yl]butan-1-ol); (587) BRL-54443 (3-(1-methylpiperidin-4-yl)-1H-indol-5-ol); (588) batoprazine; (589) benzylpiperazine; (590) binospirone; (591) 1-(8-bromobenzo[1,2-b;4,5-b]difuran-4-yl)-2-aminopropane); (592) CP-809,101 (2-[(3-Chlorophenyl)methoxy]-6-(1-piperazinyl)pyrazine); (593) CP-93,129 (3-(1,2,3,6-tetrahydropyridin-4-yl)-1,4-dihydropyrrolo[3,2-b]pyridin-5-one); (594) CP-94,253 (3-(1,2,5,6-tetrahydro-4-pyridyl)-5-propoxypyrrolo[3,2-b]pyridine); (595) CGS-12066A (4-(4-methylpiperazin-1-yl)-7-(trifluoromethyl)pyrrolo[1,2-a]quinoxaline); (596) chlorophenylbiguanide; (597) chlorphentermine; (598) dazopride; (599) dimemebfe; (600) 2,5-dimethoxy-4-bromoamphetamine; (601) 2,5-dimethoxy-4-fluoroamphetamine; (602) 2,5-dimethoxy-4-methylamphetamine; (603) EMD-386,088 (5-chloro-2-methyl-3-(1,2,3,6-tetrahydro-4-pyridinyl)-1H-indole); (604) EMDT (2-(2-ethyl-5-methoxy-1H-indol-3-yl)-N,N-dimethylethanamine); (605) p-fluoropiperazine; (606) fluprazine; (607) jimscaline; (608) LY-293,284 ((4R)-6-acetyl-4-(di-n-propylamino)-1,3,4,5-tetrahydrobenz[c,d]indole); (609) lasmitidan; (610) lorcaserin; (611) 2-methyl-5-hydroxytryptamine; (612) 2-methyl-4,5-methylenedioxyamphetamine; (613) NBUMP(N-[4-[4-(2-methoxyphenyl)piperazin-1-yl]butyl]adamantane-1-carboxamide); (614) 1-(1-naphthyl)piperazine; (615) Org-37,684 ((3S)-3-[(2,3-dihydro-5-methoxy-1H-inden-4-yl)oxy]pyrrolidine); (616) PNU-22394 (6-Methyl-1,2,3,4,5,6-hexahydro-azepino[4,5-b]indole)); (617) PRX-00023 (N-(3-[4-(4-cyclohexylmethanesulfonylaminobutyl)piperazin-1-yl]phenyl)acetamide); (618) RH-34 (3-[2-(2-methoxybenzylamino)ethyl]-1H-quinazoline-2,4-dione); (619) RS56812 (N-[(3R)-1-azabicyclo[2.2.2]oct-3-yl]-2-(1-methyl-1H-indol-3-yl)-2-oxoacetamide); (620) RS67333 (1-(4-amino-5-chloro-2-methoxyphenyl)-3-(1-butyl-4-piperidinyl)-1-propanone); (621) RU24969 (5-Methoxy-3-(1,2,5,6-tetrahydro-4-pyridinyl)-1H-indole); (622) Ro60-0175 ((S)-6-Chloro-5-fluoro-1H-indole-2-propanamine); (623) TFMFly ((2R)-1-(8-trifluoromethyl-2,3,6,7-tetrahydrobenzo[1,2-b:4,5-b′]difuran-4-yl)-2-aminoethane); (624) U92016-A 48R)-8-(Dipropylamino)-6,7,8,9-tetrahydro-3H-benz[e]indole-2-carbonitrile) (625) VER3323 ((2S)-1-(6-bromo-2,3-dihydroindol-1-yl)propan-2-amine); (626) vilazodone; (627) WAY-181,187 (1-[(2S,5S)-4,4-difluoro-5-(hydroxymethyl)tetrahydrofuran-2-yl]pyrimidine-2,4(1H,3H)-dione); (628) WAY-208,466 (N′-[(2Z)-4-(2,4-dichlorophenyl)-3-(2-methylpropyl)-1,3-thiazol-2(3H)-ylidene]-2-(pyrazin-2-yloxy)acetohydrazide); (629) YM-348 (2S)-1-(7-ethyl-1H furo[2,3-g]indazol-1-yl)propan-2-amine); (630) alprenolol; (631) BMY 7378 (8-(2-[4-(2-methoxyphenyl)-1-piperazinyl]ethyl)-8-azaspiro[4.5]decane-7,9-dione); (632) cyanopindolol; (633) iodocyanopindolol; (634) lezcotozan; (635) methiothepin; (636) NAN-190 (1-(2-methoxyphenyl)-4-(4-phthalimidobutyl)piperazine); (637) oxprenolol; (638) pindolol; (639) propranolol; (640) robalzotan; (641) S15535 (1-(2,3-dihydro-1,4-benzodioxin-8-yl)-4-(2,3-dihydro-1H-inden-2-yl)piperazine); (642) spiperone; (643) TFMPP; (644) UH-301 ((S)-5-fluoro-8-hydroxy-2-(dipropylamino)tetralin); (645) WAY-100,135 ((S)—N-tert-butyl-3-(4-(2-methoxyphenyl)-piperazin-1-yl)-2-phenylpropanamide); (646) WAY-100,635 (N-[2-[4-(2-methoxyphenyl)-1-piperazinyl]ethyl]-N-(2-pyridyl)cyclohexanecarboxamide); (647) mefway; (648) 5-hydroxytryptophan; (649) 5-hydroxytryptophan creatinine sulfate complex; (650) 5-methoxytryptamine; (651) 5-methoxytryptamine creatinine sulfate complex; (652) 5-HIAA (5-hydroxyindoleacetic acid); and (653) 5-HIAA (5-hydroxyindoleacetic acid) creatinine sulfate complex; and the salts, solvates, analogues, congeners, bioisosteres, hydrolysis products, metabolites, precursors, and prodrugs thereof.

34. The composition of claim 1 wherein the third agent is melatonin or a salt thereof.

35. The composition of claim 12 wherein the second agent is a non-steroidal anti-inflammatory drug and the third agent is selected from the group consisting of serotonin sulfate, a serotonin creatinine sulfate complex, serotonin hydrochloride, and melatonin or a salt thereof.

36. The composition of claim 35 wherein the first agent is selected from the group consisting of metformin or a salt thereof, phenformin or a salt thereof, buformin or a salt thereof, and a butyrate compound selected from the group consisting of a butyrate salt and a butyrate ester.

37. The composition of claim 36 wherein the first agent is selected from the group consisting of metformin or a salt thereof and a butyrate salt.

38. The composition of claim 12 wherein the first agent is an adenosine 5′-monophosphate-activated protein kinase (AMPK) activator and the third agent is selected from the group consisting of serotonin sulfate, a serotonin creatinine sulfate complex, serotonin hydrochloride, and melatonin or a salt thereof.

39. (canceled)

40. The composition of claim 39 wherein the first agent is selected from the group consisting of metformin or a salt thereof, phenformin or a salt thereof, buformin or a salt thereof, and butyrate or a salt thereof.

41. (canceled)

42. The composition of claim 41 wherein the second agent is acetylsalicylic acid or a salt thereof, ibuprofen or a salt thereof, or celecoxib or a salt thereof.

43.-44. (canceled)

45. The composition of claim 28 wherein the first agent is an adenosine 5′-monophosphate-activated protein kinase (AMPK) activator and the second agent is a non-steroidal anti-inflammatory drug.

46. (canceled)

47. The composition of claim 45 wherein the third agent is selected from the group consisting of serotonin sulfate, a serotonin creatinine sulfate complex, serotonin hydrochloride, and melatonin or a salt thereof.

48. The composition of claim 1 wherein the composition comprises aspirin or a salt thereof, metformin or a salt thereof, and melatonin or a salt thereof.

49. The composition of claim 1 wherein the composition further comprises a pharmaceutically acceptable carrier.

50. The composition of claim 1 wherein the composition further comprises a liposome, wherein the composition is encapsulated by the liposome.

51. The composition of claim 1 wherein: the first agent is selected from the group consisting of metformin or a salt thereof and a butyrate salt; the second agent is selected from the group consisting of aspirin or a salt thereof and celecoxib or a salt thereof; and the third agent is selected from the group consisting of melatonin or a salt thereof, serotonin creatinine sulfate complex, and serotonin hydrochloride.


SEQUENCE LISTING (7 sequences)

SEQ ID NO: 1 - protein, 9 aa, Homo sapiens
SAFSVGLET

SEQ ID NO: 2 - protein, 6 aa, Homo sapiens
PIRFTK

SEQ ID NO: 3 - protein, 16 aa, Homo sapiens
FYNQQNHYDGSTGKFH

SEQ ID NO: 4 - protein, 11 aa, Homo sapiens
NIPGLYYFAYH

SEQ ID NO: 5 - protein, 17 aa, Homo sapiens
TVYMKDVKVSLFKKDKA

SEQ ID NO: 6 - protein, 22 aa, Homo sapiens
TYDQYQENNVDQASGSVLLHLE

SEQ ID NO: 7 - protein, 13 aa, Homo sapiens
GDQVWLQVYGEGE